(19) United States
(12) Patent Application Publication
(10) Pub. No.: US 2004/0106163 A1
(43) Pub. Date: Jun. 3, 2004

(54) Non-invasive measurement of analytes

(76) Inventors: Jerome James Workman JR., Brookline, MA (US); Christopher Robert Lambert, Hudson, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/617,915

(22) Filed: Jul. 10, 2003

Related U.S. Application Data
(60) Provisional application No. 60/425,488, filed on Nov. 12, 2002.
(60) Provisional application No. 60/438,837, filed on Jan. 9, 2003.

(51) Int. Cl. C12Q 1/54

(52) U.S. Cl. 435/14

(57) ABSTRACT

This invention provides devices, compositions and methods for determining the concentration of one or more metabolites or analytes in a biological sample, including cells, tissues, organs, organisms, and biological fluids. In particular, this invention provides materials, apparatus, and methods for several non-invasive techniques for the determination of in vivo blood glucose concentration levels based upon the in vivo measurement of one or more analytes or parameters found in skin.

### RELATED APPLICATIONS

This invention is a continuation in part of U.S. Ser. No. 10/______, filed on Jul. 9, 2003 and claims priority to the U.S. provisional patent application serial No. 60/425,488, filed Nov. 12, 2002, and serial No. 60/438,837, filed Jan. 9, 2003, each of which is incorporated by reference in its entirety.

### FIELD OF THE INVENTION

This invention provides devices, compositions and methods for determining the concentration of one or more analytes in a biological sample, including cells, tissues, organs, organisms, and biological fluids. In particular, this invention provides materials, apparatus, and methods for several non-invasive techniques for the determination of in vivo blood glucose concentration levels based upon the in vivo measurement of one or more analytes or parameters found in skin.

### BACKGROUND OF THE INVENTION

Identifying and understanding the risk factors associated with diabetes is invaluable for the development and evaluation of effective intervention strategies.

Lacking normal regulatory mechanisms, diabetics are encouraged to strive for optimal control through a modulated life style approach that focuses on dietary control, exercise, and glucose self-testing with the timely administration of insulin or oral hypoglycemic medications. Invasive forms of self-testing are painful and fraught with a multitude of psychosocial hurdles, and are resisted by most diabetics. Alternatives to the currently available invasive blood glucose testing are highly desirable.

Conventional approaches seek to reduce or eliminate the skin trauma, pain, and blood waste associated with traditional invasive glucose monitoring technologies. In general, though never effectively demonstrated prior to this invention, noninvasive optical blood glucose monitoring requires no samples and involves external irradiation with electromagnetic radiation and measurement of the resulting optical flux. In theory, it was always hoped that glucose levels could be derived from the spectral information following comparison to reference spectra for glucose and background interferants, reference calibrants, and/or application of advanced signal processing mathematical algorithms. Radiation-based technologies often referred to as potential candidates for solving the non-invasive glucose problem have included variations of sampling and data processing methods including: 1) mid-infrared (MIR) spectroscopy, 2) near-infrared radiation (NIR) spectroscopy, 3) radio wave impedance, 4) autofluorescence and white light scattering, and 5) Raman spectroscopy. Each of these methods uses optical sensors, and relies on the premise that the absorption or fluorescence pattern of electromagnetic radiation can be quantitatively related to a change in blood glucose concentration. Other endogenous substances such as water, lipids, proteins, and hemoglobin are known to absorb energy, particularly infrared light and can easily obscure the relatively weak glucose signal.

Other approaches to non-invasive glucose measurements are based on microvascular changes in the retina, acoustical impedance, nuclear magnetic resonance (NMR) spectroscopy and optical hydrogels that quantify glucose levels in tear fluid. While putatively non-invasive, these technologies have yet to be demonstrated as viable in clinical testing.

Nearly noninvasive techniques tend to rely on interstitial fluid extraction from skin. This can be accomplished using permeability enhancers, sweat inducers, and/or suction devices with or without the application of electrical current. One device recently approved by the FDA relies on reverse iontophoresis, utilizing an electrical current applied to the skin. The current pulls out salt, which carries water, which in turn carries glucose. The glucose concentration of this extracted fluid is measured and is proportionate to that of blood. In keeping with its nearly noninvasive description, this technology is commonly associated with some discomfort and requires at least twice daily calibrations against conventional blood glucose measurements (e.g. invasive lancing).

Other nearly noninvasive blood glucose monitoring techniques similarly involve transcutaneous harvesting for interstitial fluid measurement. Other technologies for disrupting the skin barrier to obtain interstitial fluid include: 1) dissolution with chemicals; 2) microporation with a laser; 3) penetration with a thin needle; and/or 4) suction with a pump. Minimally invasive blood glucose monitoring can also involve the insertion of an indwelling glucose monitor under the skin to measure the interstitial fluid glucose concentration. These monitors typically rely on optical or enzymatic sensors. Although technologically innovative, these in situ sensors have had limited success. Implantable glucose oxidase sensors have been limited by local factors causing unstable signal output, whereas optical sensors must overcome signal obfuscation by blood constituents as well as interference by substances with absorption spectra similar to glucose. Moreover, inflammation associated with subcutaneous monitoring may contribute to systematic errors requiring repositioning, recalibration or replacement, and more research is needed to evaluate the effects of variable local inflammation at the sensor implantation site on glucose concentration and transit time.

Interstitial fluid glucose concentrations have previously been shown to be similar to simultaneously measured fixed or fluctuating blood glucose concentrations. See, e.g., Bantle et al., Journal of Laboratory and Clinical Medicine 130:436-441, 1997; Sternberg et al., Diabetes Care 18:1266-1269, 1995. Such studies helped validate noninvasive/minimally invasive technologies for blood glucose monitoring, insofar as many of these technologies measure glucose in blood as well as interstitial fluid.

A noninvasive glucose monitor that is portable, simple and rapid to use, and that provides accurate clinical information is desirable. In particular, the ability to derive first and second order information in real-time for dynamic glucose metabolism, such as the direction and rate of change of bioavailable glucose distributed within the blood and interstitial fluid space, would be extremely important for continuous and discrete glucose monitoring.

### SUMMARY OF THE INVENTION

The methods and compositions of the present invention effectively determine the glucose concentration in blood for a living organism by non-invasive, in vivo measurement of the glucose level in skin by means of fluorescence measurements of metabolic indicators/reporters of glucose metabolism. Disclosed are dyes used as metabolic indicators that allow for specific in vivo monitoring of metabolites, which are used as indicators of metabolic activity. A dye characterized by this invention is referred to herein as a small molecule metabolite reporter (“SMMR”).

This invention provides for fluorescence measurements of extracellular and intracellular reporter molecules placed into the cytosol, nucleus, or organelles of cells within intact, living, tissue that track the concentration of blood glucose in an organism. When any one of a series of metabolites is measured using this technique, the molar concentration of blood glucose can be calculated. Direct or indirect fluorescence measurements of glucose is described using one or more of the following measurements: pH (as lactate/H⁺), membrane reduction-oxidation electric potential, NAD(P)H (nicotinamide adenine dinucleotide (phosphate), reduced form) for energy transfer, FAD⁺ (flavin adenine dinucleotide, oxidized form) for energy transfer, ATP/ADP ratio, Ca²⁺-pumping rate, Mg²⁺-pumping rate, Na⁺-pumping rate, K⁺-pumping rate, and vital mitochondrial membrane stains/dyes/molecules fluorescence response. These analytes, measured in skin using the techniques taught herein, provide a complete picture of epidermal skin glycolytic metabolism where local epidermal analyte (glucose) quantities are proportional to the concentration of glucose in systemic blood, specifically the capillary fields within the papillary layer of the dermis (corium). Temperature and/or nitric oxide measurement may also be combined with the above measurements for better calibration and determination of glucose concentrations.

The invention provides methods for measuring in vivo blood glucose levels through the skin by monitoring, in a population of cells, one or more relevant metabolites, parameters or analytes in at least one metabolic pathway. The one or more metabolite(s), parameter(s) or analyte(s) is monitored by measuring the fluorescence spectrum emitted by a reporter composition located in the skin. The fluorescence spectrum emitted by the reporter is stoichiometrically related to the metabolite, parameter or analyte concentration in the population of cells. The in vivo blood glucose level is determined by analyzing the fluorescence spectrum, using the known stoichiometric relationship between the fluorescence spectrum of the reporter and the metabolite, parameter or analyte concentration.

The population of cells can have a predominantly glycolytic metabolism, or alternatively, the population of cells can be induced to have a glycolytic metabolism. The population of cells in the skin can be located in the epidermis, which contains a dynamic, metabolically homogeneous, and homeostatic population of cells. For example, the population of cells having a glycolytic metabolism can include live keratinocytes. These live keratinocytes can be present in the epidermal layer of skin. In some cases, the live keratinocytes can be present in the skin at a depth, from the surface of the skin, of about 10 μm, which corresponds to the bottom of the dead stratum corneum layer, to about 175 μm, which corresponds to the top of the dermal layer.

The metabolic pathways monitored within the population of cells, according to these methods for measuring in vivo blood glucose levels through the skin, can be monitored by measuring a specific metabolite or analyte of the glycolytic pathway, wherein the specific metabolite or analyte has a known stoichiometric or highly correlated relationship with glucose concentration. The metabolic pathways can also be monitored within the population of cells by observing a physico-chemical parameter that is related to the glycolytic pathway, wherein the selected physico-chemical parameter has a stoichiometric or highly correlated relationship with glucose concentration.

For example, the relevant metabolites or analytes that are monitored in these methods for measuring in vivo blood glucose levels through the skin can be lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; inorganic phosphate (Pᵢ); glycogen; pyruvate; nicotinamide adenine dinucleotide phosphate, oxidized form (NAD(P)+); nicotinamide adenine dinucleotide (phosphate), reduced form (NAD(P)H); flavin adenine dinucleotide, oxidized form (FAD); flavin adenine dinucleotide, reduced form (FADH₂); or oxygen (O₂) utilization.

The population of cells to be monitored in these methods for measuring in vivo blood glucose levels through the skin can have a predominantly oxidative metabolism, or alternatively, the population of cells can be induced to have a metabolism predominantly based on oxidative phosphorylation. The metabolic pathways monitored within the population of cells can be monitored by measuring a metabolite or analyte that is generated as a result of the oxidative metabolic pathway, wherein the specific metabolite or analyte has a stoichiometric or highly correlated relationship with glucose concentration. Alternatively, the metabolic pathways can be monitored within the population of cells by observing a physico-chemical parameter that is generated as a result of the oxidative metabolic pathway, wherein the physico-chemical parameter has a stoichiometric or highly correlated relationship with glucose concentration.

The invention also provides skin sensor compositions that can be present in the epidermis at a depth, from the surface of the skin, of about 10 μm, which corresponds to the bottom of the dead stratum corneum layer, to about 175 μm, which corresponds to the top of the dermal layer. The skin compositions are present in the epidermis at an effective concentration that allows one or more metabolites or analytes in a metabolic pathway to be detected in a subject or biological sample. In one case, the skin sensor composition can include a reporter dye and a marker dye. In this case, the marker dye is used as a reference wavelength for the reporter dye, which changes emission at only one wavelength in response to glucose. Alternatively, the skin sensor composition can include a dye that exhibits a wavelength shift in absorption or fluorescence emission in the presence of a metabolite, such as, for example, glucose. In this second case, only one dye is used as the SMMR, because a first emission wavelength of the fluorescence spectrum increases with glucose, while a second emission wavelength decreases. The ratio of the first and second emission wavelengths can be determined, thereby allowing the selected dye to act as a self-referencing SMMR.

The reporter dye used in the skin sensor compositions of the invention can be a mitochondrial vital stain or dye, or a dye exhibiting one or more of a redox potential, an energy transfer properties, or a pH gradient. Suitable mitochondrial vital stains or dyes include, but are not limited to, a polycyclic aromatic hydrocarbon dye such as, for example, rhodamine 123; di-4-ANEPPS; di-8-ANEPPS; DiBAC₄(3); RH421; tetramethylrhodamine ethyl ester, perchlorate; tetramethylrhodamine methyl ester, perchlorate; 2-(4-(dimethylamino)styryl)-N-ethylpyridinium iodide; 3,3′-dihexyloxacarbocyanine, 5,5′,6,6′-tetrachloro-1,1′,3,3′-tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; or iodine dissolved in potassium iodide. The reporter dye can also be coumarin; derivatives of coumarin, anthraquinones; cyanine dyes, azo dyes; xanthene dyes; arylmethine dyes; pyrene derivatives; or ruthenium bipyridyl complexes.

The one or more metabolite(s) or analyte(s) to be detected in a subject or biological sample include, for example, lactate; hydrogen ion (H⁺); calcium ion (Ca²⁺) pumping rate; magnesium ion (Mg²⁺) pumping rate; sodium ion (Na⁺) pumping rate; potassium (K⁺) pumping rate; adenosine triphosphate (ATP); adenosine diphosphate (ADP); the ratio of ATP to ADP; glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); flavin adenine dinucleotide, reduced form (FADH₂); or oxygen (O₂) utilization.

An effective concentration of the skin sensor composition is, for example, at least between 1 to 500 μg/ml, between 5 to 150 μg/ml, and 10 to 100 μg/ml. The SMMR can be introduced in a low concentration in a range from 10 μM to 500 μM and in a volume from 200 μL to 0.1 μL, respectively (e.g., introducing the SMMR at a concentration in the range of 200 μL of a 10 μM SMMR solution to 0.1 μl of a 500 μM SMMR solution). One specific application of the skin sensor composition is, for example, a 5 μL volume of a 400 μM SMMR solution, or a 10 μL volume at 200 μM concentration.

The one or more metabolite(s) or analyte(s) can directly report on, and relate to, in vivo blood glucose levels. Suitable metabolites or analytes include any of the metabolites or analytes listed herein.

The invention also provides methods for monitoring the concentration of one or more metabolite(s) or analyte(s) in a metabolic pathway using the skin sensor compositions of the invention. According to these methods, the skin sensor composition is applied to the surface of the skin for a predetermined period of time. The skin sensor composition penetrates the epidermis to a depth of about 10 μm, which corresponds to the bottom of the dead stratum corneum layer, to about 175 μm, which corresponds to the top of the dermal layer. An optical reader is used to monitor changes in the concentration of the one or more metabolite(s) or analyte(s) in a metabolic pathway. These changes in concentration are monitored by detecting changes in one or more reporter dyes, at one or more points in time. Monitoring the change in metabolite or analyte concentration can be accomplished by detecting at least one wavelength above 450 nm.

The skin sensor composition used in these methods for monitoring the concentration of one or more metabolite(s) or analyte(s) can include, for example, a mitochondrial stain sensitive to membrane potential or chemical gradient. Examples of suitable mitochondrial stains include a polycyclic aromatic hydrocarbon dye, such as, for example, rhodamine 123; di-4-ANEPPS; di-8-ANEPPS; DiBAC₄(3); RH421; tetramethylrhodamine ethyl ester, perchlorate; tetramethylrhodamine methyl ester, perchlorate; 2-(4-(dimethylamino)styryl)-N-ethylpyridinium iodide; 3,3′-dihexyloxacarbocyanine, 5,5′,6,6′-tetrachloro-1,1′,3,31 -tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; or iodine dissolved in potassium iodide.

The skin sensor compositions can also include a dye or stain that transfers energy from a molecule that is generated as a result of the oxidative metabolic pathway, wherein the molecule has a stoichiometric or highly correlated relationship with glucose concentration. The skin sensor composition can also include coumarin; derivatives of coumarin; anthraquinones; cyanine dyes; azo dyes; xanthene dyes; arylmethine dyes; pyrene derivatives; or ruthenium bipyridyl complexes.

The skin sensor compositions used in these methods for monitoring the concentration of one or more metabolite(s) or analyte(s) can be formulated as emulsions, ointments, disposable gel film patches, reservoir devices, creams, paints, polar solvents, non-polar solvents, or any combination thereof.

Penetration of the skin composition to a depth of about 10 μm to about 175 μm can be accomplished using an active transport technique or a passive transport technique, such as, for example, electroporation, laser poration, sonic poration, ultrasonic poration, iontophoresis, mechanical-poration, solvent transport, tattooing, wicking, or pressurized delivery. In addition, penetration of the skin sensor composition to the desired depth can be accomplished by combining the composition with various molecular size attachments.

The predetermined amount of time during which the skin sensor composition is applied to the surface of the skin can be, for example, at least 24-48 hours, at least 2-6 hours, from about 5 seconds to 5 minutes, and from about 30 seconds to 5 minutes.

The invention also provides methods for monitoring in vivo blood glucose levels by applying the skin sensor compositions of the invention to a surface of the skin for a predetermined period of time. The skin sensor compositions penetrate the epidermis to a depth of about 10 μm, which corresponds to the bottom of the dead stratum corneum layer, to about 175 μm, which corresponds to the top of the dermal layer. An optical reader is used to detect changes in the reporter dye by monitoring changes in the concentration of the one or more metabolites or analytes. The change in the concentration of the one or more metabolites or analytes is then correlated with in vivo blood glucose levels. Monitoring the change in metabolite or analyte concentration can be accomplished by detecting at least one wavelength above 450 nm.

The skin sensor composition can include a mitochondrial vital stain or dye, or a dye exhibiting redox potential or energy transfer properties. Suitable mitochondrial vital stains or dyes include at least one polycyclic aromatic hydrocarbon dye, such as, for example, Rhodamine 123, Di-4-ANEPPS; Di-8-ANEPPS, DiBAC₄(3), RH421, Tetramethylrhodamine ethyl ester, perchlorate, Tetramethylrhodamine methyl ester, perchlorate, 2-(4-(dimethylamino)styryl)-N-ethylpyridinium iodide, 3,3′-Dihexyloxacarbocyanine, 5,5′,6,6′-tetrachloro-1,1′,3,3′-tetraethyl-benzimidazolylcarbocyanine chloride, 5,5′,6,6′-tetrachloro-1,1′,3,3′-tetraethyl-benzimidazolylcarbocyanine iodide, Nonylacridine Orange, Dihydrorhodamine 123 and Dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; and iodine dissolved in potassium iodide. The skin sensor composition can include coumarin, derivatives of coumarin, anthraquinones, cyanine dyes,-azo dyes, xanthene dyes, arylmethine dyes, pyrene derivatives, or ruthenium bipyridyl complexes.

The skin composition can be formulated as emulsions, creams, ointments, disposable gel film patches, reservoir devices, paints, or solvent mixtures.

The invention also provides sensor systems that include a device having a component that transmits radiation to a material or tissue, a component that detects radiation emitted from the material or tissue, and a component to display the detection results. The sensor systems further include an applicator that delivers the skin sensor compositions of the invention to the material or tissue. Typically, there is an air interface between the device and the material or tissue, wherein the air interface measures the resulting excitation radiation emitted from the irradiated skin sensor composition.

The device included in the sensor system can emit radiation at one or more wavelengths that have been chosen to specifically excite the skin composition that is applied to the material or tissue. The skin sensor composition can include a reporter dye and a marker dye, or alternatively, a dye exhibiting a wavelength shift in absorption or fluorescence emission in the presence of a metabolite. The skin sensor composition can be present at a depth from the surface of the skin of about 10 μm to about 175 μm in the epidermis in a concentration that is effective for detection of one or more metabolites or analytes in a biological sample.

The sensor system can detect radiation at one or more wavelengths that have been chosen to specifically identify fluorescence emission that has been scattered back to the system from the skin sensor composition.

The invention also provides methods for determining blood glucose concentration. According to these methods, an instrument response measurement is performed using a calibration target, and the response data is recorded. A dye mixture is applied to the skin in a first, small controlled spot, such that the dye resides in the epidermal layer of the skin, and a second dye mixture is applied to the skin in a second, small controlled spot. The second spot is perturbed, such that the extreme changes that the mixture may undergo are achieved. A calibration measurement is then performed on the perturbed spot, and the calibration data is recorded. A background measurement is made on an area of skin that has no dye, this background data is recorded. A measurement on the first spot is performed by illuminating the first spot with light, and the wavelength spectrum of light reflected back from the first spot is detected. Further measurements on the first spot are performed at wavelengths suitable for each dye present. A parameter from the response data is calculated in order to normalize the background, calibration and measurement data for the response of the spectrometer. A parameter from the background data is calculated in order to correct the calibration and measurement data for emission, absorption and scattering properties of the tissue. A metabolite parameter from the calibration data is calculated in order to relate the measurement data to the blood glucose concentration.

The invention also provides methods of calculating a blood glucose concentration. According to these methods, a background response and an autofluorescence tissue response is measured from a calibration target that includes an epidermal layer of skin. A first dye is provided to a first skin location, and residues of the first dye mixture are transferred into the epidermal layer of the skin. A second dye is provided to a second skin location, and at least one extreme change in the mixture is triggered and recorded. The extreme change can be, for example, a change in concentration of the analyte comprising a zero or low concentration and a saturation level or high concentration. These extremes are used to calibrate the sensor enabling it to measure a test sample accurately with a concentration between the extremes. See e.g., equations (13) through (21), as described herein. The first skin location is illuminated with a radiative emission, and a resulting wavelength spectrum reflected from the first skin location is detected. The illuminating and detecting can be repeated using irradiation and wavelength spectra associated with each dye provided. At least one physico-chemical parameter that is related to the glycolytic pathway is then detected. Preferably, the physico-chemical parameter has a stoichiometric or highly correlated relationship with glucose concentration, which is used in determining the blood glucose concentration. The sensor system can include a bloodless calibration procedure such as, for example, the procedure(s) outlined in equations 13, 16, 17, 18, 19, 20 or 21 set forth herein.

The invention also provides methods for determining the concentration of at least one metabolite or analyte in skin tissue. According to these methods, a small molecule metabolite reporter (SMMR) agent is administered to the skin tissue. The SMMR agent penetrates to a region of the skin at a depth between the dermis and the epidermis, wherein the depth from the surface of the skin is from about 10 μm to about 175 μm. The SMMR agent is irradiated with a source of electromagnetic radiation, and the fluorescence spectra emitted from the SMMR agent is detected. The emitted fluorescence spectra are then analyzed, which results in a determination of the concentration of the metabolite or analyte. Measuring the fluorescence spectra according to these methods can include a bloodless calibration procedure, such as, for example, the procedure(s) outlined in equations 13, 16, 17, 18, 19, 20 and 21 set forth herein.

Unless otherwise defined, all technical and scientific terms used herein have the same meanings as commonly understood by one of ordinary skill in the art to which the invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Other features and advantages of the invention will be apparent from the following detailed description and claims.

### BRIEF DESCRIPTION OF THE DRAWINGS

FIG. 1 is a schematic showing the preferred location for coloring skin cells using a skin SMMR composition of the invention, wherein the Reporter and Marker colors are introduced into the stratum germinativum near the surface of the skin.

FIG. 2A and FIG. 2B are schematics showing the method for coloring epidermal skin cells (i.e., keratinocytes) of the fingertip (FIG. 2A) using a skin SMMR composition of the invention, wherein one or more SMMRs applied to the skin surface are transported for up to 50 microns (μm) through the top of the skin using passive or active transport (FIG. 2B).

FIG. 3A and FIG. 3B are schematics showing the fluorescence response to D-glucose using a Lactate/H⁺ small molecule metabolite reporter (FIG. 3A), and the corresponding epidermal location of the SMMR in the stratum germinativum near the surface of the skin (FIG. 3B) and demonstrates a spectral response to changes in D-glucose as measured by lactate/H⁺ reporting shown in FIG. 3A.

FIG. 4A and FIG. 4B are schematics showing a measurement technique for determining D-glucose concentrations utilizing one or more wavelengths. FIG. 4A depicts Reporter and Marker channel detection using a dual wavelength measurement technique. FIG. 4B depicts measurement of the Total Integrated Fluorescence Signal (gray region). The initial signal measured to determine glucose concentration [Glucose_{I}] is derived as a function of the ratio of the fluorescence signal from the reporter to marker such that [Glucose_{I}]=f(Reporter/Marker). A multichannel wavelength correction is applied later. As designated in the FIG. 4A, FL*=Fluorescence detection.

FIG. 5A and FIG. 5B are schematics showing a broad wavelength correction technique for correcting the fluorescence ratio. Corrected signal for Glucose concentration [Glucose_{C}] is a function of the ratio of reporter to marker signal corrected for variation in reflection (i.e., broad wavelength reflection) unique for each individual, such that [Glucose_{C}]=f(Reporter/Marker)×(Broad Wavelength Reflection Correction). FIG. 5A depicts light correction profile detection. FIG. 5B depicts broad wavelength reflection signal measurement (gray area). As designated in the FIG. 5A, DR*=Diffuse Reflection.

FIG. 6 is a flow chart showing signal processing logic for determining glucose levels. The Detector signal (as fluorescence or diffuse reflectance) is pre-amplified and the initial glucose calculation is made. One or more of a series of Demographic functions (e.g., empirical modeling of different Demographic clusters of the population of diabetics, as shown in the figure) are applied to the initial glucose calculation. A physiological correction is then further applied, as well as a glucose model to derive the corrected blood glucose computation (i.e., glucose concentration).

FIG. 7 is a flow chart showing determination of glucose concentration. The Detector signal (as fluorescence or diffuse reflectance) is pre-amplified and the total fluorescence counts are determined. The initial glucose calculation is made and is corrected using the diffuse reflection information as per FIGS. 5A and 5B. Demographic and Physiology functions (e.g., empirical modeling of different Physiological clusters of the population of diabetics, as shown in the figure) are then applied to correct for individual skin optical properties and unique physiology. The corrected glucose levels are then subjected to a final correction model relating measured skin glucose to blood glucose (lag correction). The result is a blood glucose computation derived from a measurement of skin fluorescence.

FIG. 8 is a schematic showing blood glucose concentration determination using measured fluorescence ratio versus D-glucose. The measured ratio response versus D-glucose changes as a function of changing blood glucose concentration. Also shown is the corresponding relative lactate/H⁺ concentration.

FIG. 9A and FIG. 9B are schematics showing blood glucose concentration results determined for actual versus measured SMMR ratios for a timed rat-clamp study. Blood glucose concentration determination using measured fluorescence ratio versus blood D-glucose ranges from 118 to 249 mg/dL blood D-glucose concentration obtained using the YSI method (YSI Incorporated, PO Box 279, Yellow Springs, Ohio 45387 USA) (FIG. 9A). Glucose is infused at 2:28, 25 g/dL at 7.5 ml/hr. The results of this study are plotted in FIG. 9B on a standard Clarke Error grid showing all data points from the experiment in Region A (center diagonal labeled “A”) having 6.76% total error, 1 sigma. As shown, the Clarke Error grid analysis divides the correlation plot into five regions. Region A represents glucose values that deviate from the comparative value by <20%, or are <70 mg/dL when the comparative value is <70 mg/dL. The B regions (broader center diagonal labeled “B”) represent values that deviate by greater than 20%, and if heeded would lead to benign treatment. Deviations within Regions A and B are considered clinically acceptable. Region C (mid-axis near top and bottom labeled “C”) values are described as those deviations that would overcorrect an acceptable glucose. Region D (mid-axis left and right labeled “D”) consists of those deviation values that would result in a dangerous failure to detect and treat a blood sugar condition. Region D values below 70 mg/dL are particularly common among the majority of consumer use glucose measurement devices. Many home blood glucose meters have up to 20% of data points in the D region. Region E deviations (left vertical and bottom labeled “E”) are described as those points that if heeded would result in a potentially erroneous and dangerous treatment.

FIG. 10A and FIG. 10B are a schematic and a magnified insert, respectively, showing how small metabolites present in the blood are transported from the blood vessels of the dermis into the interstitial fluid of the epidermis. This occurs as the metabolites move from small blood vessels in the subcutaneous layers of the integument into the capillary fields of the dermis. Metabolite molecules useful for tracking glucose include D-glucose, lactate; H⁺; NAD(P)H; Ca²⁺; FAD⁺; redox potential (mitochondrial membrane); ATP/ADP; and O₂ (aerobic). Such small metabolite molecules move from the capillaries to the interstitial fluid surrounding the epidermal keratinocytes via mass transport. Thus the metabolite concentration for interstitial fluid outside the keratinocytes is proportional to the concentration of metabolites in peripheral dermal blood vessels. The only exception to this from the list of useful metabolite molecules is oxygen, which decreases with distance from the subcutaneous blood vessels. At approximately 50 to 100 μm down from the surface of the skin, there is very little oxygen, which is why the keratinocytes must function using anaerobic glycolysis. Applications of SMMRs as reporters for blood metabolite and precursor levels can be inferred from peripheral tissue metabolite levels (i.e., why measurements of skin are useful for measurement of some blood metabolites). Small metabolite molecules move from the capillaries to the interstitial fluid via non-insulin regulated, concentration dependent, mass transport (i.e., a diffusion rate of ˜4 to 10% per minute of the difference in concentration between capillary and skin metabolite levels). The skin cells transport via GluT1 (GenBank Accession Number: K03195), not GluT4 (GenBank Accession Number: M91463).

FIG. 11 is a schematic showing the placement of at least one SMMR into a keratinocyte. SMMRs are added to the skin surface with, e.g., a disposable patch, and are passively or actively transported to a keratinocyte. Indirect mechanisms 1-3 and direct mechanisms 4-5 for fluorescence measurement are further detailed in FIGS. 12-16.

FIG. 12 is a schematic of SMMR mechanism 1 for an indirect energy transfer reporter. SMMR energy transfer reporter mechanism for fluorescence signal detection is based upon energy transfer from a metabolite molecule to the SMMR. The metabolite molecule is excited and transfers energy to the SMMR. Then, the emission from the SMMR is detected with a sensor. Under conditions where energy transfer is a significant route for the decay of the excited metabolite, and where there is present a non-rate-limiting excess of SMMR, the emission intensity is then proportional to the concentration of metabolite present.

FIG. 13 is a schematic of SMMR mechanism 2 for an indirect metabolite reporter. SMMR metabolite reporter mechanism for a fluorescence signal is based upon the influence of a metabolite molecule on the SMMR. The fluorescent SMMR is excited wherever the influence of the metabolite alters the fluorescence properties of the SMMR. This altered fluorescence emission from the SMMR is detected with a sensor. Where there is a non-rate-limiting excess of SMMR, the emission intensity is proportional to the concentration of metabolite present.

FIG. 14 is a schematic of SMMR mechanism 3 for an indirect membrane potential reporter. SMMR membrane potential reporter mechanism for a fluorescence signal is based upon the fluorescence properties of an SMMR when bound to a cellular membrane, such as the inner membrane of mitochondria. A fluorescent SMMR is excited wherever the influence of the membrane potential at the membrane-binding site alters the fluorescence properties of the SMMR. This altered fluorescence emission from the SMMR is detected with a sensor. Where there is a non-rate-limiting excess of SMMR, the emission intensity is proportional to the concentration of metabolite present.

FIG. 15 is a schematic of SMMR mechanism 4 for a direct complex intensity reporter. SMMR direct complex intensity reporter mechanism for a fluorescence signal is based upon the specific binding of a metabolite molecule (e.g., D-glucose) into a larger protein (e.g., enzyme-based) SMMR. The fluorescent protein SMMR is excited wherever the influence of the specifically bound metabolite alters the fluorescence properties of the SMMR. This altered fluorescence emission from the SMMR is detected with a sensor. Where there is a non-rate-limiting excess of SMMR, the emission intensity is proportional to the concentration of metabolite present. This mechanism is effective for intracellular, extracellular, and in vitro glucose quantitative measurements. This mechanism could also be useful for in vitro diagnostic use.

FIG. 16 is a schematic of SMMR mechanism 5 for a direct complex lifetime reporter. SMMR direct complex lifetime reporter mechanism for an absorption signal is based upon the specific binding of a metabolite molecule (e.g., D-glucose) into a larger protein (e.g., enzyme-based) SMMR. The protein-based SMMR is excited by irradiation using modulated light, whereas irradiation with a second wavelength of light is used to monitor the transient absorption lifetime using a detection system that can include a lock-in amplifier. The influence of the specifically bound metabolite such as glucose alters the excited state lifetime properties of the SMMR. This altered lifetime from the SMMR is detected with a sensor. Under conditions where the influence of the metabolite is significant, and where there is a non-rate-limiting excess of SMMR, the fluorescence lifetime signal is proportional to the concentration of metabolite present. As in the case of mechanism 4, this mechanism is effective for intracellular, extracellular, and in vitro glucose quantitative measurements. It would be obvious to one skilled in the art that this mechanism could also be useful for in vitro diagnostic use.

FIGS. 17A, 17B, 17C and 17D are schematics depicting mechanisms operating in skin metabolism, which are referred to as Scheme 1, Scheme 2 and Scheme 3, respectively. FIG. 17A depicts mechanisms operating in skin metabolism and points of measurement using SMMRs (Scheme 1). FIG. 17B depicts an overview of the metabolic pathways for glucose in epidermis (Scheme 2). FIG. 17C depicts the structure of a generic chemical backbone for designing a pH sensitive dye for specific action as a lactate/H+ SMMR (Scheme 3). FIG. 17D illustrates fluid issues related to in vivo skin calibration (Scheme 4).

FIG. 18 is a schematic of glycolysis showing the specific analytes where glucose measurements are made for the invention. SMMRs are used by measuring glucose directly, or by measuring metabolites as indirect indicators of the quantity of glucose entering the cellular glycolytic pathway. Such metabolites are described in detail for the invention and examples are given here as: reducing equivalents molecules (e.g., NAD(P)H, NADH, FAD, FADH₂); changes in ATP-driven processes (e.g., cation pumping, transport at membranes, membrane reduction-oxidation electric potential, and pH gradient); and stoichiometric products of glucose utilization in glycolysis (e.g., lactate, hydrogen ion, pH, and pyruvate).

### DETAILED DESCRIPTION

In vivo fluorescence (autofluorescence) has been used for a number of years to determine the metabolic state and to monitor pharmaceutical effects in cells and tissues. See, e.g., Dellinger et al., Biotechnol Appl Biochem, 28 (Pt. 1): 25-32, (1998). Consideration of the photophysics involved in autofluorescence rapidly leads one to the conclusion that the use of autofluorescence alone, as the analytic probe or mechanism, imposes some severe limitations on any measurement technique.

Recently, the state-of-the-art in making time resolved fluorescence measurements have advanced to a degree whereby robust and low-cost instrumentation can be readily assembled. However, so far, effective measurements have only been made in vitro for specific analytes, and real-time in vivo analysis has yet to be reported. Researchers have used phase-modulation fluorometry in vitro to demonstrate first generation sensing devices for a number of analytes (pO₂, pH, pCO₂, NH₃, etc.). See e.g., Dalbey, R. E., et al., J. Biochem. Biophys. Meth., 9: 251-266, (1984). The use of long lifetime red-sensitive probes has also allowed for transdermal sensing to become a reality since human skin is translucent at wavelengths above 630 nm. Lifetime-based sensing offers novel applications in the bioprocessing and biomedical arenas. Measurement of Green Fluorescent Protein (GFP) as a marker for expression of heterologous proteins does not require any additional co-factors for its visualization. GFP-fusion proteins have been expressed in a variety of cell lines and in situ measurements in bioreactors have been made. Fluorescence polarization measurements for the quantitation of large antigens, such as antibodies labeled with long-lived fluorescent labels, can, in principle, directly measure antigens of several million Daltons (Da).

Fluorescence techniques are capable of detecting molecular species at picomole (pm) levels or less. This sensitivity arises because of the simplicity of detecting single photons against a dark background. This advantage disappears if there are other fluorescent species in the detection volume that is obtained from the sample material being measured. Furthermore, fluorescence intensity is not an absolute technique and measurements must be referenced to an internal standard using a ratiometric or comparative method.

Autofluorescence arises from the innate fluorescence of compounds that are not particularly efficient fluorophores and that are not photostable. Because of these properties, detectors for autofluorescence need to have an excellent signal-to-noise ratio, with sufficient dynamic range, and require that the excitation source be of low enough power so as not to cause photosensitization. In addition, there are a number of fluorescing species present in the skin that constitute a significant background signal. The situation is further complicated in that it is quite difficult to identify or introduce a standard optical reference material or apparatus into the skin.

It is well known that specific dyes bind to cellular structures and allow imaging and anatomical/histological studies of intracellular structures. See, e.g., the information available from companies such as Molecular Probes or Sigma-Aldrich. It is also well known that some signals from these dyes can be used to characterize cellular metabolism in vitro. Fluorescent chemical sensors have been reported to play a critical role in the elucidation of cellular mechanisms by giving real-time information about the environment of a cell in a non-destructive manner. See, e.g., Glass, J. Am. Chem. Soc. 2000, 122: 4522-4523. For these applications, sensor affinity and selectivity are of utmost concern, Thus, a useful sensor must recognize its analyte with high specificity and possess an affinity that is commensurate with the average concentration of the analyte in solution. Id. However, no specific fluorophores have been named, and no fluorophore design requirements have been published for in vivo, non-invasive elucidation of metabolic pathways for any medical applications in general, and, specifically, for those described by this invention (i.e., measurement of blood glucose). A surprising discovery has been made that the detailed and specific absorption and emission spectral characteristics of a select set of dyes, when introduced into living cells of organisms (in vivo), change as a qualitative and quantitative indication of extracellular and intracellular metabolism. One or more dyes of the select set presented herein are specifically used to report metabolite concentration, which are then used to further define the quantity or quality of metabolic activities within living organisms, such as glycolysis.

This invention most specifically relates to small molecule metabolite reporters (SMMRs) that indicate the rate and quantity of glycolysis occurring within the living cell loci. The detailed spectral changes noted as direct and indirect metabolic reporters include: variation in fluorescence emission intensity and lifetime, variation in wavelength position for absorption and emission maxima, and variation in bandwidth and spectral shapes of absorption and emission spectra. These measurable changes vary in direct proportion to the changes in concentrations of metabolite molecules within the physical proximity of associated extracellular and intracellular structures. The information provided by measuring the changes in specific reporter dye spectra following introduction into living organisms has led to a low-cost method and apparatus for the detailed, real-time measurement and delineation of metabolic pathways and processes in living organisms. When molecules are used in the method for providing in vivo metabolite reporting as described herein, they are referred to herein as “small molecule metabolite reporters” or SMMRs.

A dye that is classified as an SMMR meets several minimum criteria: SMMRs have low toxicity; they can be delivered precisely to target tissue; they report quantitative information with respect to the concentration of specific metabolites when measured in vivo; and they are fluorescent.

In order to qualify as a SMMR according to this invention, dyes require one or more of the following criteria:

1. Enhancement of signal-to-noise ratio of native autofluorescence measurements through the process of:

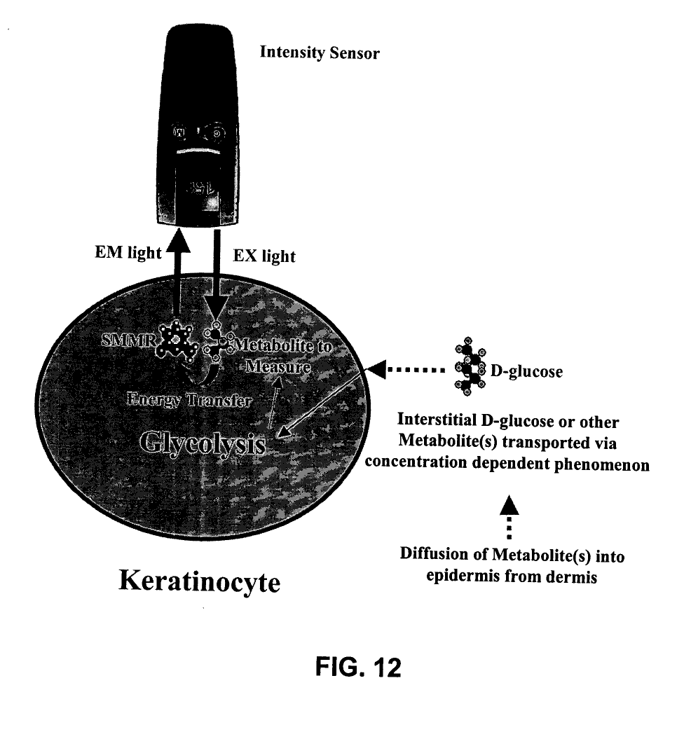

ENERGY TRANSFER from NADH, NAD(P)H, or FAD⁺ to SMMRs (which boosts signal by 5 to 50 fold) that is an indirect indication of redox transfer coenzyme activity within cells and tissues due to glycolysis (see FIG. 12; Mechanism 1);

2. Enhancement of Specific Metabolite and Precursor Signals such as:

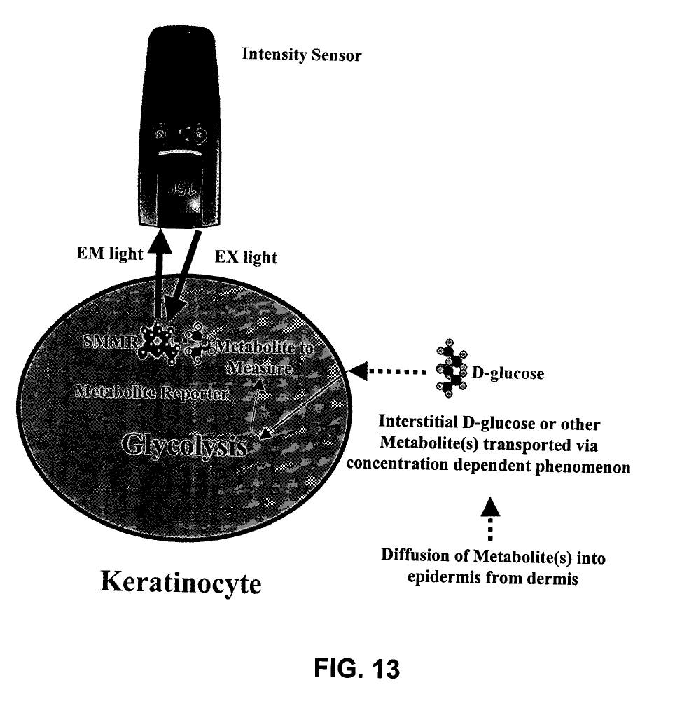

a. Lactate SMMRs that indicate lactate/hydrogen ion formation from anaerobic glycolysis activity (measurement sites include intracellular, extracellular, and organelle loci) (see FIG. 13; Mechanism 2);

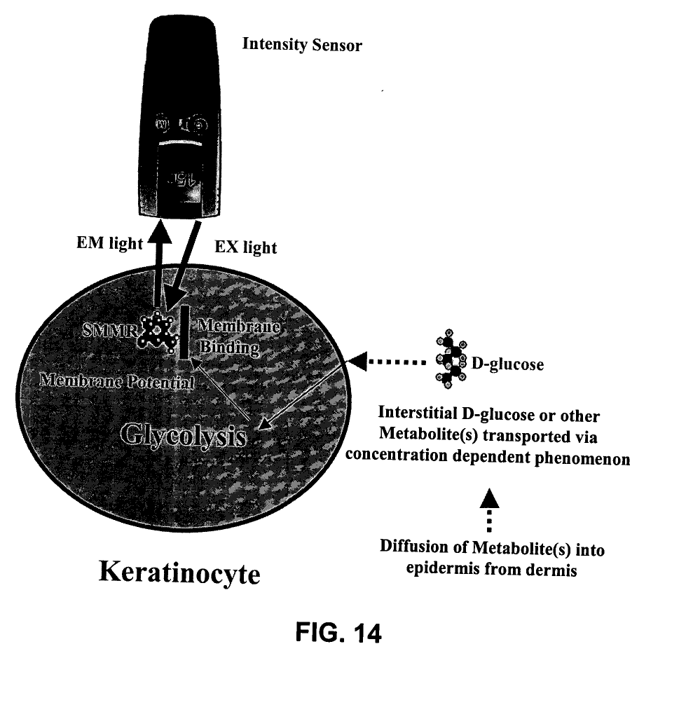

b. Mitochondrial Membrane Potential SMMRs that indicate overall changes in mitochondrial membrane redox-potential that corresponds to changes in glucose (see FIG. 14; Mechanism 3);

c. Calcium ion (Ca²⁺) tracking SMMRs that indicate available adenosine triphosphate (ATP) and ion pump transport activity fueled by glycolytic activity (see FIG. 13; Mechanism 2);

d. Glycogen SMMRs using glycogen-staining molecules that indicate the occurrence of glycolysis and resultant storage of glycogen molecules (see FIG. 13; Mechanism 2).

3. Direct measurement of glucose molecules in vivo using:

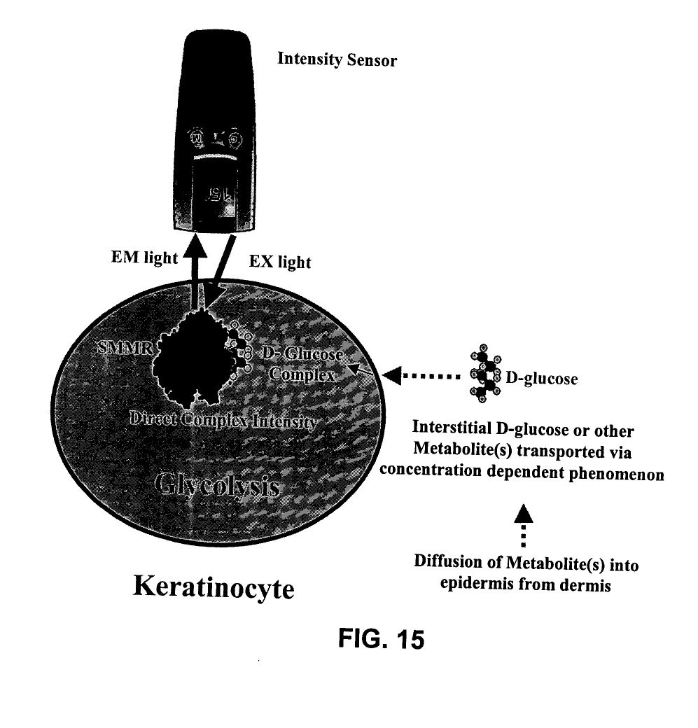

a. Protein-labeled fluorophores such as proteins that are specifically bound to glucose and have enhanced fluorescence quantum efficiency. When placed into the skin, the resulting fluorescence is indicative of the amount of glucose present (see FIG. 15; Mechanism 4);

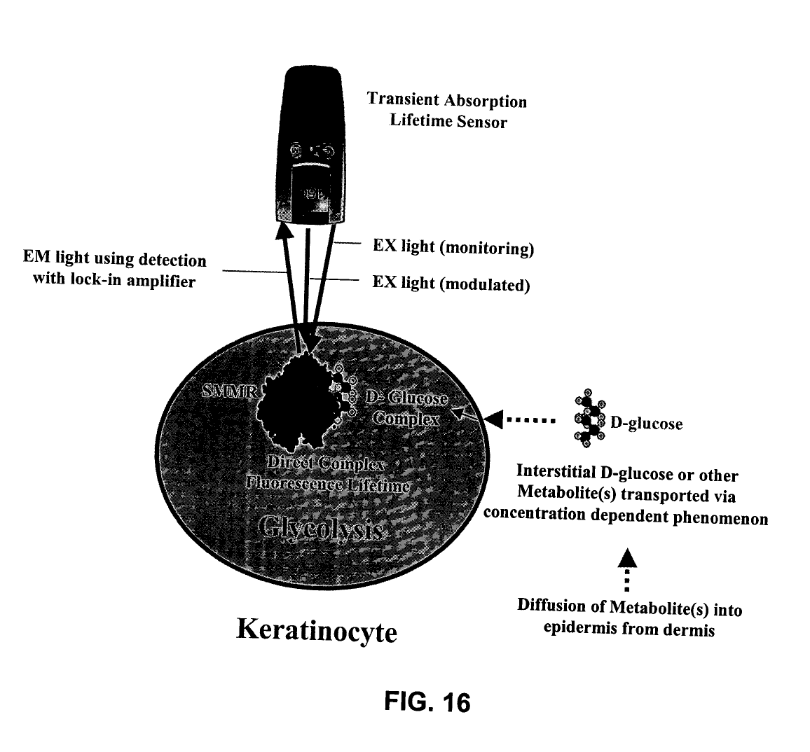

b. Proteins with a photoredox active cofactor (such as flavin adenine dinucleotide, i.e., FAD) that are used to observe excited state lifetime fluorescence by monitoring the triplet state of FAD (³FAD*) (see FIG. 16; Mechanism 5).

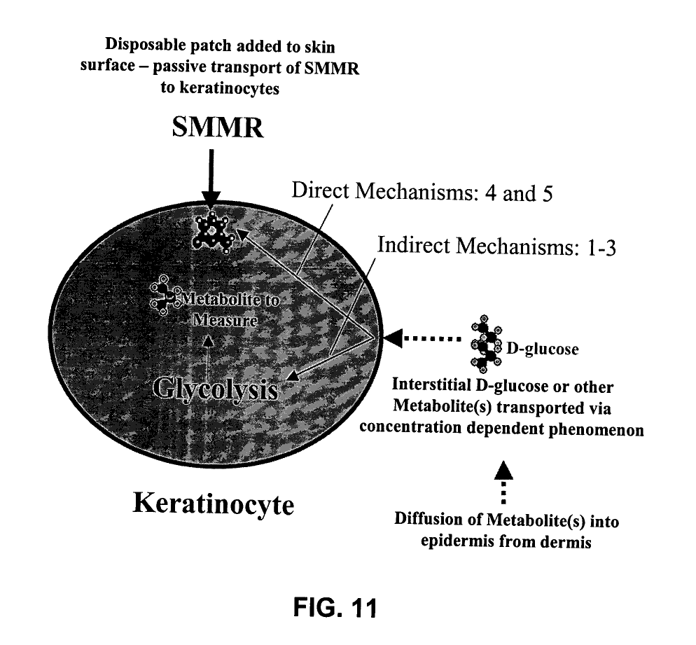

These mechanisms are referred to as Mechanisms 1-5 and are depicted schematically in FIGS. 11-16. Table 1 below provides a summary of these methodologies, which depict several overall exemplary SMMR mechanisms for detection of metabolites using changes in fluorescence response.

Required mechanisms for energy transfer using SMMRs, such as depicted in FIG. 12, include but are not limited to a singlet bimolecular electronic energy transfer (ET) reaction that can be designated as B*+A→B+A*, where the energy is transferred from molecule B (metabolite) to A (SMMR). Such an energy transfer takes place by one or more of the following energy transfer mechanisms:

a. Long-range resonance energy transfer (a.k.a., Fluorescence Resonance Energy Transfer (FRET) or Förster transfer), which is a transfer of energy from a metabolite fluorophore to a SMMR fluorophore as a result of a dipolar coupling between adjacent fluorophores. This transfer occurs between molecules over a distance of up to 5 nanometers (nm);

b. Short-range collisional energy transfer (CET), which requires electron-exchange interactions between the donor and acceptor molecular orbitals (that is the main mechanism of transfer in the majority of SMMRs);

c. Static quenching in which the donor and acceptor molecules are in close proximity in the ground state; and,

d. Radiative energy transfer (RET), involving donor emission and reabsorption of the photon by the acceptor. RET is often referred to as a trivial mechanism for ET.

### Table 1

1.0 Enhancement of Signal-to-Noise of native autofluorescence (INDIRECT)

1.1 Energy Transfer from NADH, NAD(P)H, or FAD to Reporters (boosts signal by 5 to 50) indicating redox transfer coenzyme activity within cells and tissues (Mechanism 1; FIG. 12)

2.0 Enhancement of Specific Metabolite and Precursor Signals (INDIRECT)

2.1 Lactate Reporters indicate lactate formation from anaerobic glycolysis activity (intracellular, extracellular, and organelle) (Mechanism 2; FIG. 13)

2.2 Mitochondrial Membrane Potential Reporters indicates overall mitochondrial membrane potential (Mechanism 3; FIG. 14)

2.3 Ca²⁺ Reporters indicate available ATP and ion pump transport activity fueled by glycolytic activity (Mechanism 2; FIG. 13).

3.0 Glucose Reporters indicating quantitative levels of D-glucose (DIRECT)

3.1 Protein-Labeled Fluorophores (Mechanism 4; FIG. 15)

3.2 Proteins with a photoredox active cofactor (such as FAD) to observe 3FAD* (Mechanism 5; FIG. 16)

Mechanisms for identifying and/or constructing exemplary SMMRs of the invention are described below. Mathematical models are provided based on the metabolite or metabolic pathway to be analyzed. In many embodiments, the SMMR is a fluorescent reporter dye. In addition, certain exemplary SMMRs of the invention are available commercially, and include, but are not limited to, the following: (1) Rh123 for measuring NAD(P)H (nicotinamide adenine dinucleotide (phosphate), reduced form) using energy transfer, or FAD⁺ (flavin adenine dinucleotide, oxidized form) using energy transfer; (2) membrane localizing dyes such as diphenylhexatriene, xanthenes, cyanines as well as diphenyl hexatriene and its derivatives, for measurement of energy and glucose transport by membrane receptors such as GluT1; (3) pH (i.e., lactate/H⁺) indicating dyes such as phenolphthalein, xanthene dyes such as 2′,7′-bis-(2-carboxyethyl)-5-(and-6)-carboxyfluorescein, (BCECF), benzenedicarboxylic acid, 2(or 4)-[10-(dimethylamino)-3-oxo-3H-benzo[c]xanthene-7-yl]-(SNARF-1) for calculations of lactate/H+ ratios, cytosolic NAD/NADH ratios or pyruvate/lactate ratios; (4) dyes known to have altered emission properties depending on the redox potential, ATP/ADP ratio, Ca²⁺-pumping rate, Mg²⁺-pumping rate, Na⁺-pumping rate, or K⁺-pumping rate of its surroundings, as these processes are ATP regulated and ATP formation in keratinocytes is a direct result of glycolysis fueled by glucose; (5) vital mitochondrial membrane stains or mitochondrial membrane dyes, especially those that produce a fluorescence response to changes in mitochondrial membrane potential; (6) reactive molecules that directly or inversely correlate to glucose concentration, such as nitric oxide (NO); and (7) molecules that directly bind to D-glucose producing a fluorescence response.

One skilled in the art may surmise that the SMMRs and methods of the invention have both in vitro and in vivo applications. However, a unique advantage of using SMMRs in clinical diagnostic and treatment applications is that their spectral response measurements are made in vivo, a distinct improvement over current in vitro analysis.

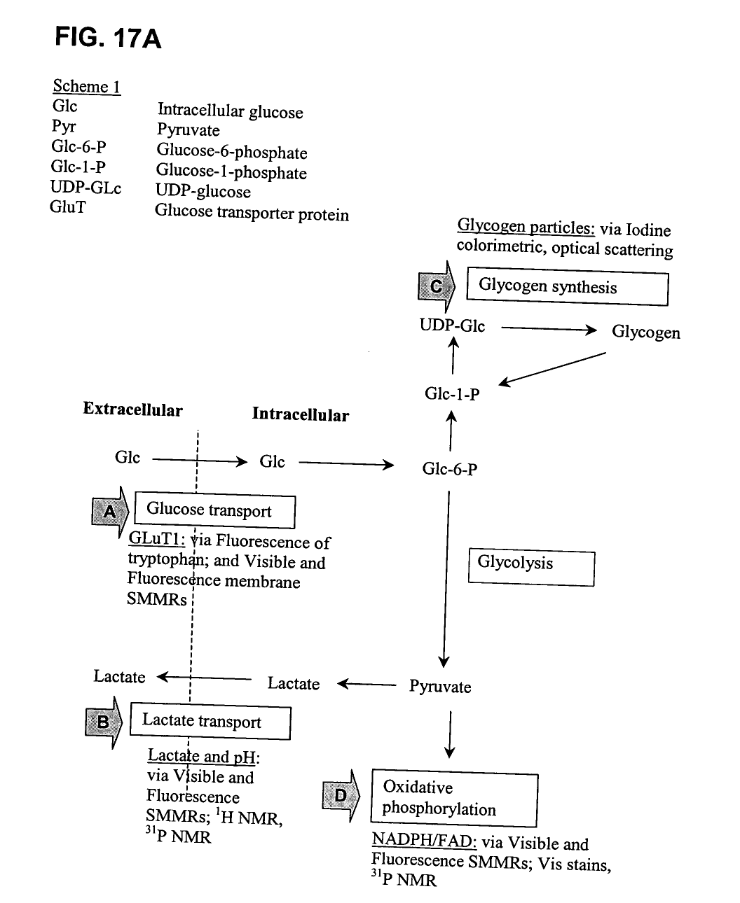

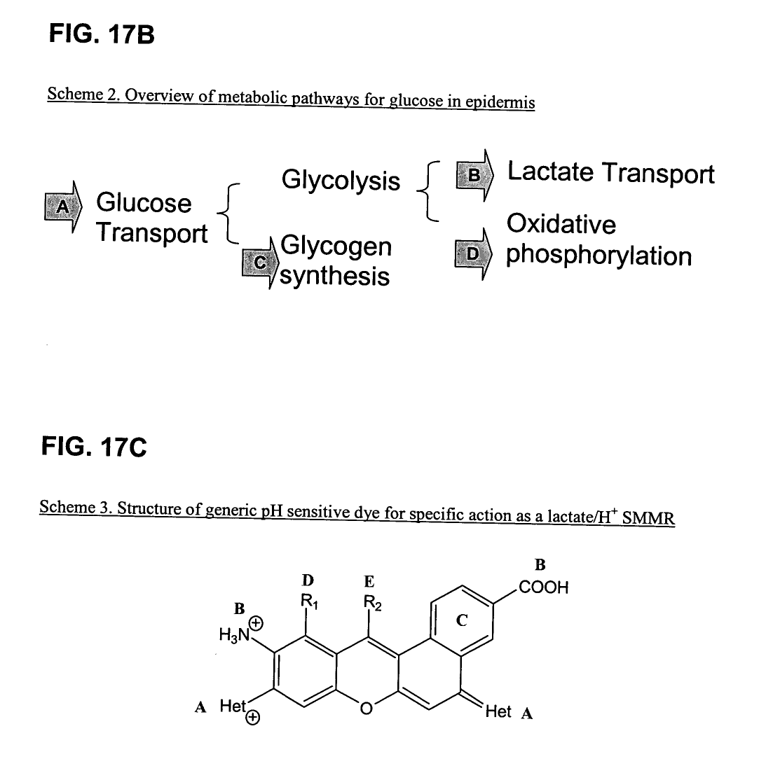

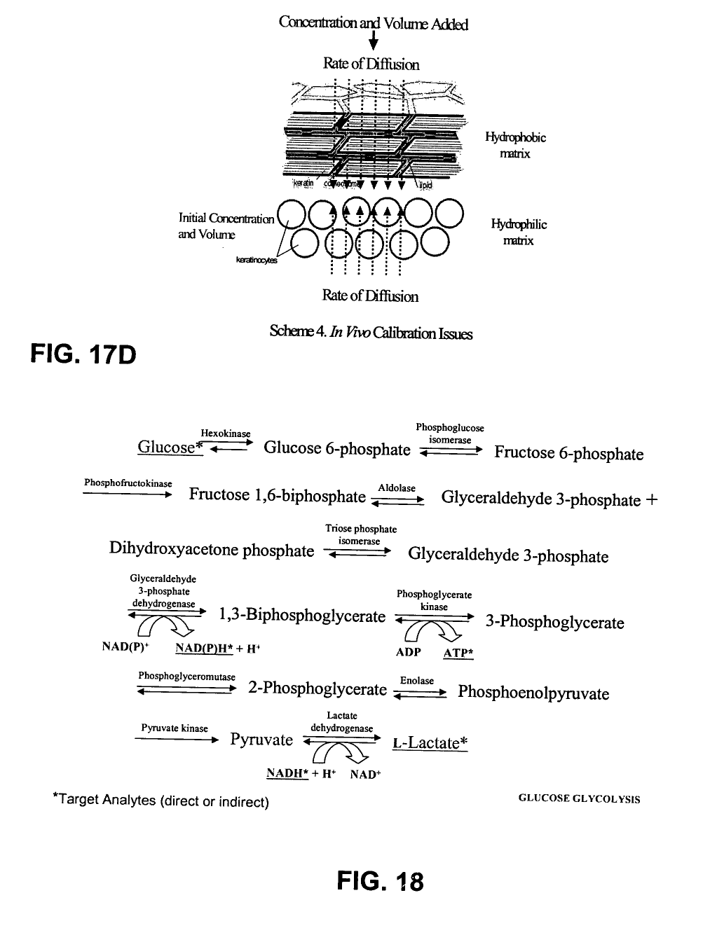

In vivo SMMR measurements require the in situ interaction of living cells with the SMMR molecules to give an accurate and real-time indication of the metabolic state for a whole organism, an organ, a tissue type, or individual cells. The measurements of the metabolic state for living organisms can thus be made non-destructively and non-invasively using spectroscopic measurements on living tissues and cells. Furthermore, custom molecules can be synthesized based on detailed understanding of SMMR interactions with in vivo metabolic processes. See, e.g., FIGS. 17A through 17C. This discovery has led to further work that allows optimization of these dye molecules in their active role as SMMRs, for reduced toxicity, selective residence time in targeted tissues, cellular binding site specificity, analyte selectivity and sensitivity, photostability, and fluorescence spectral characteristics. These fluorescence spectral characteristics can be selected based on molecular structures for SMMRs, which include: emission intensity and lifetime, location of excitation/absorption and emission maxima, Stokes shift, bandwidth, spectral shape changes due to the presence of metabolites, quantum yield, and quantum efficiency.

Therefore, this discovery is a vast improvement over current techniques such as antibody:antigen labeling, because it relates explicitly to a unique use of small molecules capable of penetrating the stratum corneum, that when placed in living tissue allow a measurable fluorescence response proportional to metabolic changes in living cells, tissues, and whole organisms (e.g., animals and humans); but without initiating an immune response. These measured metabolite signals provide delineation of metabolic pathways by measuring the spectra of certain dye molecules when the molecules are used in precise ways, under exacting conditions, and when placed in specific structures within living cells and tissues.

Mitochondrial stains have been used in vitro for measuring glucose concentration in immortal cell lines by fluorescence. See, e.g., N. Borth, G. Kral, H. Katinger, Cytometry 14:70-73 (1993). However, no known previous work determines the glucose concentration in blood for a living organism by non-invasive, in vivo measurement of the glucose level in skin by means of fluorescence measurements of metabolic indicators/reporters (such as SMMRs) of glucose metabolism.

Mitochondrial vital stains are particularly useful as SMMRs. A preferred mitochondrial vital stain or dye is a polycyclic aromatic hydrocarbon dye, including, but not limited to: rhodamine 123; di-4-ANEPPS, di-8-ANEPPS; DiBAC₄(3); RH421; tetramethylrhodamine ethyl ester, perchlorate; tetramethylrhodamine methyl ester, perchlorate; 2-(4-(dimethylamino)styryl)-N-ethylpyridinium iodide; 3,3′-dihexyloxacarbocyanine, 5,5′,6,6′-tetrachloro-1,1′,3,3′-tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene dyes, especially, 2′,7′-bis-(2-carboxyethyl)-5-(and-6)-carboxyfluorescein; and benzenedicarboxylic acid, 2(or 4)-[10-(dimethylamino)-3-oxo-3-H-benzo[c]xanthene-7-yl]; and iodine dissolved in potassium iodide. Other dyes or stains that are useful as SMMRs include, but are not limited to, fluorescein-based compounds; coumarin; derivatives of coumarin; anthraquinones; cyanine dyes; azo dyes; xanthene dyes; arylmethine dyes; pyrene derivatives; and ruthenium bipyridyl complexes.

In some cases, the measurement of temperature may be combined with direct or indirect fluorescence measurements of glucose using one or more of the following parameter measurements: pH (as lactate and/or H⁺); redox potential; inorganic phosphate (Pᵢ); glycogen; pyruvate; nicotinamide adenine dinucleotide phosphate, oxidized form (NAD(P)⁺); nicotinamide adenine dinucleotide (phosphate), reduced form (NAD(P)H); flavin adenine dinucleotide, oxidized form (FAD⁺) for energy transfer; flavin adenine dinucleotide, reduced form (FADH₂) for energy transfer; adenosine triphosphate (ATP); adenosine diphosphate (ADP); the ATP/ADP ratio; Ca²⁺-pumping rate; Mg²⁺-pumping rate; Na⁺-pumping rate; K⁺-pumping rate; oxygen (O₂) utilization and vital mitochondrial membrane stains/dyes/molecules fluorescence response. Accurate direct or indirect in vivo measurement of glucose concentration in immortal cell lines, human keratinocyte cell cultures, and mammalian (including human) skin are achieved by using this application of in vivo fluorescence labeling and detection of SMMRs in skin. These analytes measured in skin using the techniques taught herein give a complete picture of epidermal skin glycolytic metabolism where local epidermal analyte (glucose) quantities are proportional to the concentration of glucose in systemic blood, specifically the capillary fields within the papillary layer of the dermis (corium). The control of temperature at the measurement site, or the additional measurement of temperature, can be useful to correct measured fluorescence for optical pathlength, vasodilatation, perfusion, and local physiology.

The fluorescence measurement of extracellular and intracellular reporter molecules placed into the cytosol, nucleus, or organelles of cells within intact, living, tissue will track the concentration of blood glucose in an organism. When any one of a series of analytes or metabolites is measured using this technique, the molar concentration of blood glucose can be calculated. Fluorescence measurements of metabolite reporters described for this invention in a metabolic pathway of interest can be taken from one or more of the following parameters: pH (e.g., as lactate/H+); redox potential; NAD(P)H (nicotinamide adenine dinucleotide phosphate, for the reduced form using energy transfer); FAD⁺ (flavin adenine dinucleotide, for the oxidized form using energy transfer); ATP/ADP ratio; Ca²⁺-pumping rate; Mg²⁺-pumping rate; Na⁺-pumping rate; K⁺-pumping rate; and redox potential of mitochondrial and other cellular membranes. Those skilled in the art will recognize that FAD and FADH₂ are formed in the citric acid cycle during aerobic (oxidative) biosynthesis and are used for electron transport in this pathway. FIG. 18 shows anaerobic glycolysis, where NAD(P)H and NADH are the major electron donors for reductive biosynthesis.

The fluorescence response using the SMMRs according to the invention can be used for in vivo measurement of glucose concentration in immortal cell lines, human keratinocyte cell cultures, and mammalian (including human) keratinocytes of the skin epidermis. The accurate measurement of these metabolites (analytes) within epidermal tissue using this fluorescence labeling mechanism provides a complete picture both of epidermal glucose concentrations and systemic blood glucose concentrations. When SMMRs are placed within the epidermis, their fluorescence properties efficiently and accurately report on skin glycolytic metabolism, thus providing a measure of local cellular glucose quantities that are proportional to the concentration of glucose in systemic blood. The computation of blood glucose levels from a measure of skin glucose is possible due to the proportionality between systemic blood glucose concentration and the concentration of glucose in the epidermis of living organisms.

Measurement of these specific analytes and metabolites, individually or combined with ancillary measurements, provides detailed information describing glucose metabolism in living tissue. The specific invention delineated here relates to the determination of blood glucose levels based upon skin glucose levels for use in the monitoring and control of diabetes mellitus. A description of the metabolic pathways for glucose in dermis and epidermis is helpful to provide a basis for this present invention. Mechanisms operating in skin metabolism are shown in Scheme 1 of FIG. 17A. An additional overview scheme is provided in FIG. 17B. This present invention models systemic blood glucose levels based upon the application of specific first principle mathematical models to direct non-invasive fluorescence measurements made using SMMRs placed within the skin.

This invention targets in vivo measurement of analyte/metabolites that provide detailed information for epidermal glycolytic pathways that are driven specifically by D-glucose, fructose, galactose and other simple sugars, but are unaffected by molecules similar to D-glucose that are not metabolically active. Such non-active metabolites include L-glucose and other levorotatory optical isomers, or enantiomeric forms of simple or complex sugars. This in fact is used as an efficacy test for the action of glycolytic reporting SMMRs. For complex glycolytic processes such as the biosynthesis of NAD(P)H, or for glycolytic processes that are distinctly non-linear, more than one pathway can be combined to enhance analytical information content to model glucose concentration. The additional information provided by monitoring more than one metabolite is used to improve analytical performance for monitoring glucose. In this way, a final measurement system provides for a wide dynamic range for glucose and is less prone to measurement errors caused by potential interferences.

Although Scheme 1 (FIG. 17A) shows that the substrate for oxidative phosphorylation is glucose-derived, this pathway may also be fueled by lipid metabolism. This issue is not a concern when monitoring glycolysis fueled by glucose for human or mammalian epidermal keratinocytes, since this metabolic pathway is not relevant to the invention for glucose measurement in keratinocytes as only two percent of skin metabolism comprises this alternative lipid pathway, whereas 70% of assimilated glucose is metabolized by glycolysis, which is a metabolic process that derives energy for the cell exclusively from the metabolism of glucose. This and other details of skin metabolism can be found, e.g., in Johnson and Fusaro. The Role of the Skin in Carbohydrate Metabolism in: Advances in Metabolic Disorders, R. Levine (Ed.), Academic Press, 1972, 60, 1-55.

The supply of glucose in the blood both diffuses and is actively transported into the cytosol of epidermal cells. The rate of transport into the epidermis is indicative of the differential concentration of skin glucose levels and blood glucose levels. The rate of transport into skin allows for an accurate first principles mathematical extrapolation of blood glucose levels.

Once modeled, the kinetics of blood glucose transport to the skin from the blood supply of subcutaneous blood vessels enables the determination of the precise first principles mathematical relationship between the rate of change of skin glucose and the rate of change of blood glucose. Thus, rapid up or down changes in blood glucose concentration can be accurately tracked by knowing the skin glucose mean concentration levels and the rate of change of skin glucose levels. First principles mathematical models can be developed, preferably for individual patients, more preferably for small local populations, and most preferably for the universal patient case.

The invention provides at least one skin composition that includes endogenous chromophores and exogenous fluorophore/reporters (i.e., SMMRs as molecules that fluoresce as an indication of metabolic rate or by an increase in metabolite levels). By convention, factors routinely affecting the glycolytic velocity assumption set for quantitative analysis of metabolites, including lactate/H⁺, are as follows: (1) pH generally has a small effect at less than 5% relative change between pH 7 and 8; (2) temperature has a small metabolic effect at semi-controlled temperatures (e.g., 25° C. to 27° C.); (3) enzyme/coenzyme concentration is normally in excess to allow glycolysis over all physiological ranges of glucose; (4) cellular substrate concentrations are normally in excess to allow glycolysis over all physiological ranges of glucose; (5) anaerobic/aerobic ratio for target cells of interest (e.g., epidermal keratinocytes) is assumed constant per individual; and (6) cell maturity is relatively constant and assumed to be constant over the gradient of the epidermis.

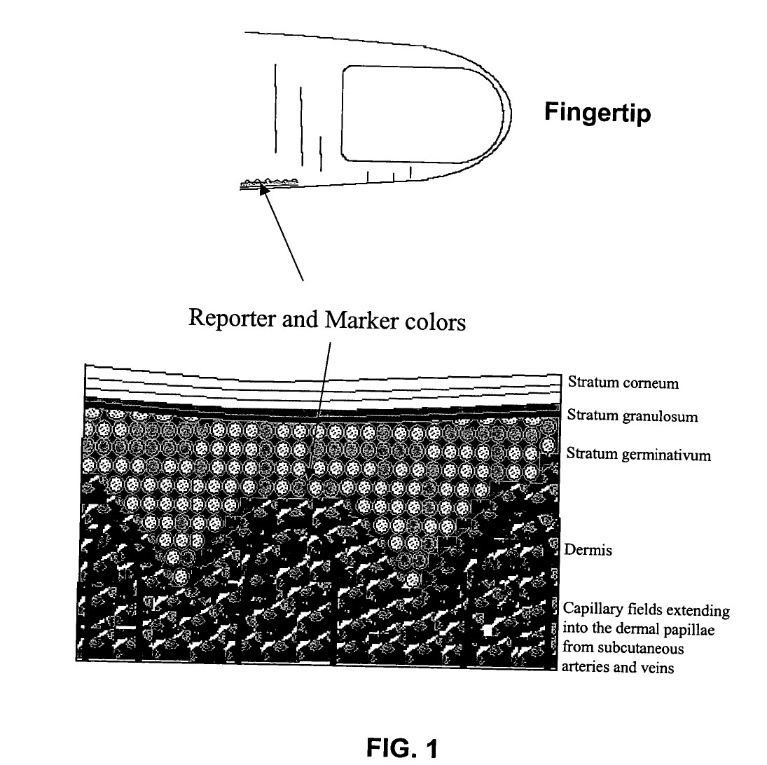

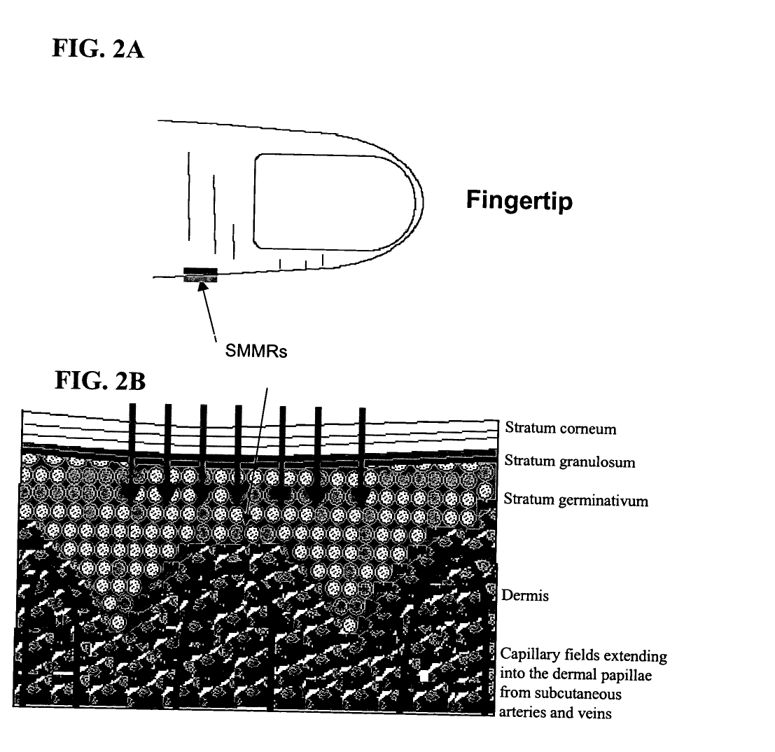

For human keratinocytes in situ, a specific layer of the epidermis (above the dermal papillae and within or above the stratum basale) is in a comparatively homeostatic condition and the major metabolic biosynthetic process is anaerobic glycolysis. This layer of cells is referred to as the stratum germinativum. Therefore, cells in the stratum germinativum make an ideal location for the introduction of SMMRs into the skin. See FIGS. 1-3 and 10. Other tissues favorable for use in the methods and compositions of the invention include all those having predominantly anaerobic glycolysis as the main biosynthetic process for glucose utilization. Thus, the epidermis throughout the human skin and at all locations becomes a prospective target site for the invention. Other epithelial tissues lining cavities within the body are also target cells for the invention. These tissues include: Simple Epithelium, e.g., squamous, cuboidal, and columnar; Stratified Epithelium, e.g., squamous, cuboidal, columnar, and transitional; and Pseudostratified Epithelium. Preferred sites for measurement application include, but are not limited to, the fingertip, the volar forearm, the upper arm, the foot, or any location where easy access to the skin is obtained without the need to disrobe.

The transport of glucose into the cell is non-insulin regulated, and the stoichiometry of anaerobic glycolysis provides two lactate/H+ molecules per one glucose molecule. Thus, intracellular lactate/H+ measurement provides the basis for inferring interstitial fluid glucose concentration in normal keratinocytes. The direct in vivo intracellular measurements of intermediate or end-product metabolites (analytes) resulting from glycolysis within keratinocytes are thus used to infer glucose substrate concentrations within the cell in real-time without the use of invasive techniques. Endogenous, native fluorophores are not considered useful reporters of metabolic state due to low signal-to-noise and to background interferences, but they do provide information about the optical properties of the tissue and the integrated history of premature tissue glycosylation that occurs over time due to the diabetic condition. Future advances in measurement technologies will likely provide accurate means for measuring autofluorescence and for relating these fluorophores to glucose concentration in tissues. However, due to the limitations in technical developments of photonic components, a more enhanced signal is required to make low-cost measurements at this time.

In contrast, the exogenous molecules described herein that are added as SMMRs to the skin result in fluorescent signals that directly report on the type and level of metabolite present in the cell. The SMMRs described herein provide unique fluorescence signals that are of sufficient magnitude to be measured using standard, low-cost, commercial photonic components. By using SMMRs, the extracellular, intracellular, and organelle microenvironments can be accurately and specifically assessed for glycolytic function within a tissue or for an organism. The exogenous fluorophore/reporters are added to the skin and are used to locate and measure metabolites located within the epidermal layer of the living skin in situ, thereby indicating the metabolic state of the organism. In alternative embodiments, these dyes are applied through oral ingestion, or more preferably by passive or active topical administration.

Effective concentrations of SMMRs to be applied are in the range of at least 1 to 500 μg/ml, e.g. 5 to 150 μg/ml or 10 to 100 μg/ml. The concentration of SMMR used is preferably from 10 to 500 μM, more preferably from 100 to 300 μM, and most preferably from 150 to 250 μM.

The localization of the dye in the skin may be controlled by various mechanisms, including but not limited to the use of electroporation, laser or mechanical poration, iontophoresis or more generally, by passive transfer using special solvent and reporter molecule mixtures. A preferred method for small molecular weight dyes is passive transport, including wicking. Passive transport may be used to allow small molecules of typically 100 Daltons (Da) to 1000 Da to enter tissues and cells. Specific examples are provided in the section labeled “Application of the small molecule metabolite reporter(s) as SMMRs.”

In some embodiments, electroporation is used to provide an exponential decay voltage pulse to create aqueous pathways through membranes. See, e.g., Zhang et al., Biochim Biophys Acta, 1572(1): 1-9, 2002. These pathways or pores may be made large enough to allow large molecules of typically 20 kDa to 250 kDa to enter tissues and cells. In an alternative SMMR embodiment, electroporation is used to deliver metabolite reporter molecules (SMMRs) in vivo to rat epidermis directly through the stratum corneum.

Electroporation also facilitates the delivery of dyes bound to large molecules that serve as anchors such as polymer beads, large polysaccharides, or colloidal particles. These approaches are contemplated as being within the invention, but are less advantageous in that the particles are often too massive to pass through the stratum corneum without active poration or mechanical injection. Once in the skin, they do not readily dissolve or organically reabsorb into the body. Such less desirable approaches would create undesirable particles that would either remain in place indefinitely or accumulate in lymph nodes, in other circulatory cavities and/or in other organ sites.

SMMRs can be made with specific properties such that they are retained only within skin cells (keratinocytes) while they report on glycolytic activity and do not harm or affect cellular metabolism. These SMMR compounds are sloughed off after a few days, even when permanently integrated into, or attached to, keratinocyte cells. The small quantity of SMMRs that diffuse away from the epidermis are rapidly degraded within the body and are completely eliminated within a few days. In preferred embodiments, reapplication of the SMMRs is relatively easy to perform. The process of sloughing off (or desquamating) follows a normal ten-day to twenty-day (typically fourteen-day) cycle as the residence time of epidermal keratinocytes moves from the basal layer (stratum basale) to the desquamating layer of the stratum corneum. Thus, SMMRs are developed to be applied once every 2 to 3 days, preferably every 3 to 4 days, and more preferably every 5 or more days.

The methods and compositions of the invention employ the measurement of the fluorescence of metabolite reporters (SMMRs) added to the skin to monitor glycolytic metabolic processes in the skin. These processes respond to blood analyte levels and to disease states affected by glycolytic activity. Autofluorescence by itself is insufficient to monitor many analytes, particularly glucose, because it does not have the necessary signal-to-noise ratio and dynamic range to be useful (i.e., accurately measured at low cost). Instead, the instant methods and compositions replace or supplement autofluorescence measurements with measurements of exogenous molecules that act as metabolite reporters localized within the epidermis.

Each of the following aspects of the SMMR system was optimized in order to derive the methods for utilizing exogenous molecule fluorescent signals in the keratinocytes for deriving blood glucose levels. The key informational requirements and assumptions include:

1. Diffusion following the laws of mass transport is the main mechanism of transport for small molecules (including D-glucose) from blood in the dermis to the keratinocytes of the epidermal layers;

2. Human keratinocytes utilize GluT1 (GenBank Accession Number: K03195) at the cell membrane (i.e., glucose transport is not insulin or GluT4 (GenBank Accession Number: M91463) regulated);

3. Glucose transport at the keratinocytes is constant relative to the maximum velocity of molecular transport and the number of active transporters within the keratinocyte cell membrane. If these are not constant, they must be modeled based upon a first principles understanding of the events that bring about changes in the transport rate. The overall effect must allow modeling of extracellular glucose levels based upon intracellular glucose levels. Thus, the intracellular glucose concentration must be based upon a known relationship to the concentration of glucose within the interstitial fluid;

4. Keratinocytes are relatively simple cells utilizing as much D-glucose as is available at any time without changing metabolic mechanisms (they remain essentially glycolytic); and they process glucose in real-time into metabolites that are directly measurable using SMMRs;

5. There is a net NAD(P)H production via the pentose shunt from glycolysis, thereby providing a mechanism for glucose measurement by using an amplified NAD(P)H signal;

6. SMMR compounds can be synthesized to demonstrate desired performance properties based upon known characteristics of molecular structure;

7. All proposed techniques using the SMMR compounds described in this invention are adaptable to small, inexpensive measurements, such as using a handheld device;

8. pH (as lactate/H⁺), NAD(P)H, Ca²⁺, FAD⁺, ATP/ADP ratio, and redox potential can be used to directly track D-glucose concentration present in the fluid surrounding human skin keratinocyte cells;

9. For anaerobic glycolysis (i.e., the metabolism of target human skin cells or keratinocytes), pH (as lactate/H⁺), NAD(P)H energy transfer, and redox potential provide the most rapid and trackable responses to glucose. The shortest response times are from 15 seconds to 2 minutes. SMMRs utilize three separate reporting mechanisms to report for these three glycolytic metabolites, including direct reporting, energy transfer, and redox potential, respectively;

10. There is a lag time for diffusion of glucose from the capillary fields of the dermis to the cells of the epidermis of no more than approximately 5-10 minutes for highly vascularized regions of the body, such as the fingertip;

11. Intracellular, extracellular and organelle lactate/H⁺ is measured as a direct indication of D-glucose concentration of surrounding fluid, where lactate/H⁺ is an indicator of keratinocyte glycolysis;

12. Measurable D-Glucose response range for these parameters is 5 to 500-plus mg/dL;

13. Human skin cells are scavenger cells, which utilize as much D-glucose as is available at any time without changing glycolytic or transport mechanisms;

14. Commercially available dye probes are useful but not optimal. Thus, strategies for independent new molecules in this regard have been developed;

15. Reporters passively transported to the skin can last up to 4 days or more using currently known methods;

16. Direct glucose measurements are possible for small treated areas of the skin but require the use of larger SMMR compounds (i.e., 100-160 kDa or more), indicating the possible requirement for electroporation schemes;

17. Small quantities of larger SMMR compounds can be optimized for signal intensity and, thus, are useful for making glucose measurements without toxicity or irritation issues in mammals, including humans.

18. A very small reaction site (i.e., 200 to 300 microns in diameter) can be used, thereby minimizing toxicity issues;

19. SMMRs as proteins, reporters and markers are placed at desired locations at the skin surface or below, namely from 10 to 500 microns in depth from the tissue surface;

20. Reporters are easy to get into the skin using passive mechanisms, but electroporation gives enhanced signal magnitudes by factor of 2 to 3 times. Electroporation is inexpensive, but adds a degree of complexity to the method;

21. None of the tested mechanisms respond to L-glucose, thereby making the tests specific for D-glucose only. (This is the ‘gold standard’ for testing the efficacy and veracity of any glycolytic and physiologically active glucose-concentration measuring technique);

22. Simple sugars, such as D-glucose, fructose, and galactose, are the sugars of interest relative to fueling glycolysis, and all cause glycolytic activity in keratinocytes.

Specific technical and scientific terms used herein have the following meanings:

As used herein, a “small molecule” is defined as a molecule from 100 Da to 250 kDa. Molecules of this molecular weight range have a demonstrated ability for use as quantitative reporters of glucose activity.

As used herein, a “chromophore” is defined as a molecule exhibiting specific absorption or fluorescence emission when excited by energy from an external source. This is a more generic term than fluorophore.

As used herein, a “fluorophore” is defined as a molecule exhibiting specific fluorescence emission when excited by energy from an external source.

As used herein, a “dye” is defined as a molecule having large absorptivity or high quantum yield and which demonstrates affinity for certain materials or organic (cellular) structures.

As used herein, a “xanthene dye” is defined as a molecule having a xanthene-like skeletal structure, which exhibits large absorptivity and high quantum yield and which demonstrates affinity for certain materials or organic (cellular) structures.

The phrase “energy transfer from reducing equivalents (e.g., NAD/NADH, NAD(P)/NAD(P)H, FAD/FADH₂) indicating SMMRs” refers to a use of SMMRs whereby the presence of these reducing equivalents molecules, is detected by excitation of the reducing equivalents molecules from an external source, energy transfer from the reducing equivalents molecule(s) to an SMMR, and detection of the fluorescence emission at the SMMR emission wavelength.

The phrase “transmembrane redox potential indicating SMMRs” refers to the use of SMMRs to indicate the degree of reduction-oxidation electric potential occurring within cellular membranes, including such organelle structures as the inner mitochondrial membrane. In one such case, the degree of reduction-oxidation electric potential is indicated by the number of SMMR molecules bound to the inner mitochondrial membrane. In this case, SMMR binding is proportional to the membrane potential as indicated by quantitative fluorescence quenching. Thus, an increase in glucose brings about an increase in glycolysis and membrane potential, thereby reducing the fluorescence signal. This phrase refers to the generic use of SMMRs as a means for detecting intracellular reduction-oxidation electric potential.

The phrase “mitochondrion-selective vital SMMRs” refers to SMMRs that bind selectively to the inner mitochondrial membrane of living cells.

The phrase “pH:lactate/H⁺ indicating SMMRs” refers to SMMRs that report on the local intra- or extracellular environment with respect to hydrogen ion concentration, pH, or lactate.

The phrase “enzyme-based SMMR, including a fluorescent protein SMMR” refers to a protein-based SMMR that is capable of reacting directly with glucose to form a fluorescence response, whether measured directly as fluorescence emission intensity or fluorescence lifetime.

The phrase “intracellular pH sensitive SMMRs” refers to SMMRs that enter the cell membrane and report on intracellular pH within the cytosol. Other pH SMMRs are distinguished as reporting on organelle pH or extracellular pH, independent of cytosolic pH.

The phrase “extracellular pH sensitive SMMRs” refers to SMMRs that remain on the outside of the cell membrane and report on extracellular pH within the interstitial fluid or extracellular environment. Other pH SMMRs are distinguished as reporting on intracellular pH, independent of extracellular pH.

The phrase “absorption/diffuse reflection or fluorescence spectrum” refers to two types of spectra measured independently. The absorption/diffuse reflection spectrum refers to the energy reflection spectrum from a material reported in either the dimensions of reflectance or absorbance versus wavelength. The fluorescence spectrum is measured independently as the fluorescence emission intensity or the fluorescence lifetime of a fluorophore following excitation from an external source.

The phrase “molecular size attachment” refers to the molecular size in Angstroms (Å), which is proportional to molecular weight in Daltons (Da), of an attachment added as an adjunct to an SMMR.

As used herein, a “reporter” is defined as an SMMR having the property of optical or fluorescence signal proportional to the quantity of analyte in the immediate vicinity of the SMMR. Thus, as the analyte quantity increases, the fluorescence signal changes (up or down) in proportion.

As used herein, a “marker” is defined as a molecule having the property of yielding a fluorescence signal that is constant when applied to target cells or tissues. Its main purpose is for use as a reference signal channel. As such, it is applied in a ratiometric measurement for correction of a reporter signal. The variation in physiological and optical characteristics of individual subjects requires a reference channel signal to correct or normalize a reporter channel signal when the ratio of reporter to marker is used for quantitative applications.

As used herein, a “sensor” is defined as a handheld device capable of making absorption or fluorescence measurements at one or more wavelengths, and converting the ratios and sums of these measurements into analyte concentrations. These analyte concentrations are used to infer the rate or quantity of a specific metabolic process.

As used herein, a “metabolite” is defined as a substance produced by a metabolic process, such as glycolysis, which can be quantitatively measured as an indication of the rate or quantity of a specific metabolic process.

As used herein, an “analyte” is defined as a measurable parameter, using analytical chemistry, which can be quantitatively measured as an indication of the rate and quantity of a specific metabolic process. The term analyte is a generic term describing such concepts as metabolites, ions, processes, conditions, physico-chemical parameters, or metabolic results that can be used to infer the rate or quantity of specific metabolic processes.

As used herein, a “response range” is defined as an analyte range (lower and upper limits) over which a metabolic process, and its measured absorption or fluorescence signal, follow a linear or defined mathematical function.

The phrase “physico-chemical parameter” refers to a subset of broadly defined analyte parameters specifically related to the physical chemistry constants of materials. These constants can be used in combination with the measurement of other analytes to infer the rate or quantity of specific metabolic processes. Such constants refer specifically to atomic mass, Faraday constant, Boltzmann constant, molar volume, dielectric properties, and the like.

As used herein, “wicking” is defined as the flow of a liquid into a solid material via the pull of gravity, Brownian motion, adhesion, mass transport, or capillary action such that a natural movement of a liquid occurs into a solid material.

The phrases “direct metabolic reporters,” and “indirect metabolic reporters” refer to the mechanism of action of SMMRs for reporting glucose concentration. Direct metabolic reporters report the concentration of glucose directly, whereas indirect metabolic reporters report the concentration of analytes used to infer the concentration of glucose.

As used herein, an “octanol-water coefficient (K_{ow})” is defined as a measure of the extent to which a solute molecule is distributed between water and octanol in a mixture. The octanol-water partition coefficient is the ratio of a chemical's solubility (concentration) in octanol to that in water using a two-phase mixture at equilibrium.

As used herein, “toxicity” is defined as the degree or quality of being toxic or hazardous to the health and well being of human and other mammalian organisms, organs, tissues, and cells.

The phrase “specialized tattoo” or more precisely the “active viewing window” refers to an area of tissue treated with an SMMR. That area is used for viewing the fluorescence ratio measurements of the SMMR interaction with tissue, in order to directly measure, calculate, or otherwise infer the concentration of skin and blood glucose or other metabolites of interest.

As used herein, a “keratinocyte” is defined as a living cell comprising the majority of the epidermis of mammalian skin. The keratinocyte is unique in both its proximity to the surface of an organism as well as in its glycolytic behavior. The keratinocyte metabolizes glucose in such a way as to produce a number of analytes whereby the glucose concentration within the cell can be inferred.

As used herein, “Rt (in ohms)” is defined as the sum of a 5-ohm series resistor and the resistance (impedance) of the skin in parallel with a 50-ohm resistor.

As used herein, “Rskin” is defined as impedance representing a function of the electrode contact resistance, the distance between electrodes, and the applied pulse. Rskin is typically in the range of 30 to 100 kohm/cm².

As used herein, “molecular size attachments” is defined as adducts to the fluorescent moieties of SMMRs to include, but are not limited to structural modifications of fluorescence SMMRs as the additions to the fluorescence structure of: acetoxy methyl esters, chloro-methyl derivatives, alkyl chain adducts, highly charged moieties, enzyme substrate mimics, enzyme cofactor tethers, and membrane binding tethers.

As used herein, a “mammal” includes both a human and a non-human mammal (e.g., rabbit, mouse, rat, gerbil, bovine, equine, ovine, etc.). Transgenic non-human animals are also encompassed within the scope of the term.

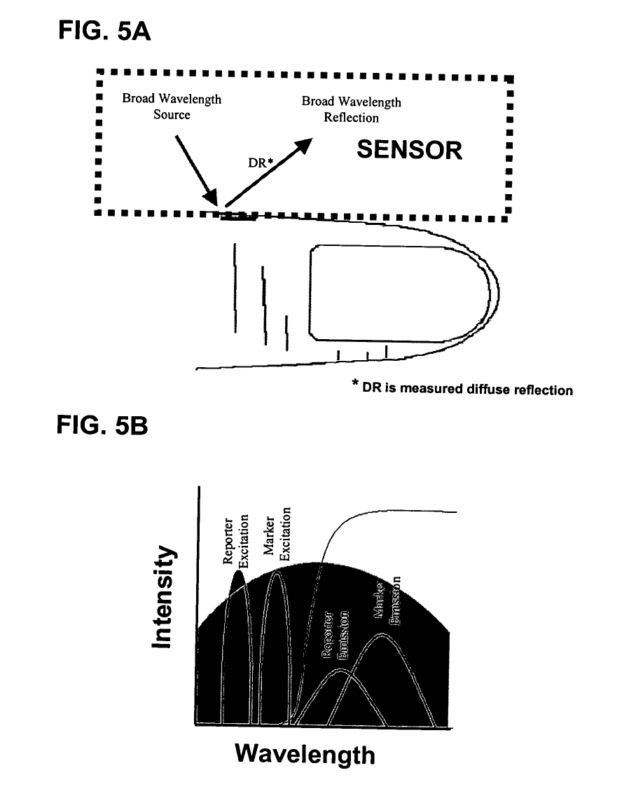

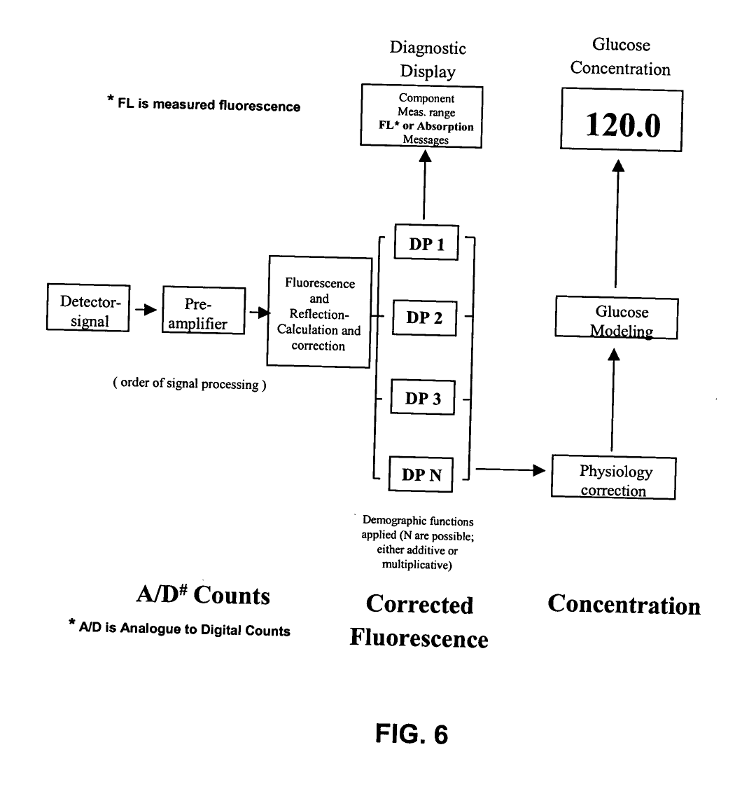

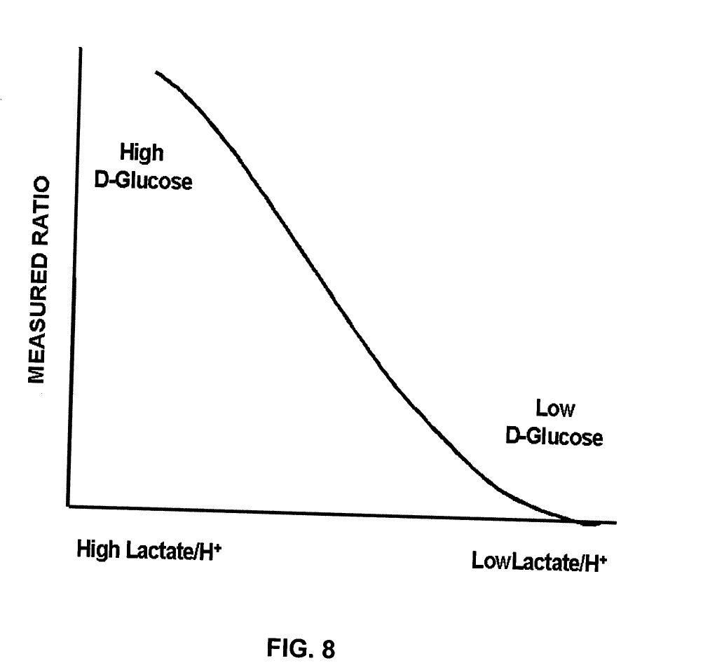

FIGS. 1-18 and Table 1 illustrate the apparatus and methods described in detail throughout the following text.

Algorithm Development and Interpretation of Data

The use of fluorescence and absorption of endogenous and exogenous chromophores and fluorophores is directed by known glycolytic metabolic pathways that operate in living tissue. The interpretation of these data and the application of the invention to the monitoring of in vivo analytes, particularly glucose, is simplified by the use of mathematical models of these metabolic processes. A number of researchers have published computer models of these processes that vary in complexity but may include: glucose transport, glycogen synthesis, lactate formation and transport, oxidative phosphorylation and the generation of reducing equivalents in tissue. These mathematical models are relevant as they allow the actual glucose concentration entering the cell to be inferred using the concentration of one or more metabolites formed during the glycolytic process. See, e.g., Jamshidi, N. et al. Bioinformatics Applications, 17(3), 2001, 286-287; Jamshidi, N. et al. Genome Research, article and publication at http://www.genome.org/cgi/doi/10.1101/gr.329302; Wiback, S. J. and Palsson, B. O. Biophysical Journal, 83, 2002, 808-818. These models are used to identify the optimum experimental conditions to measure a metabolite concentration, in particular the blood glucose concentration.

Application of the Small Molecule Metabolite Reporter(s) as SMMRs

In one embodiment, the invention provides a series of techniques that allow the placement of specialized fluorescent or absorptive molecules (SMMRs) into the epidermis using electroporation, laser poration, iontophoresis, or mechanical poration; direct application by painting; tattooing methods involving application by needle, an equivalent electrical tattooing technique; or most preferably by using passive transport. An exemplary method of passive transport is wicking. The method is comprised of a direct measurement of the fluorescence of SMMRs placed within epidermal cells, i.e., keratinocytes. This fluorescence is measured using molecules with specific properties for defining glucose metabolism in epidermis and for inferring the magnitude of the change in fluorescence signal to blood glucose concentrations.

With passive absorption, a molecule is placed on the surface of the skin and allowed to penetrate in proximity to the epidermal cells (keratinocytes) directly above the basal layer (stratum basale) at a depth from the surface of skin from 10 μm to 50 μm and up to 175 μm in the pits of the stratum basale extending into the dermis between the dermal papillae. For measurement of glucose, the placement of the SMMR is below the stratum corneum yet above the dermis, more specifically in the stratum spinosum or stratum basale immediately above the upward extensions of the dermal papillae. This SMMR placement is accomplished by varying the combination of the polarity and charge on the SMMR, the size of molecular attachments or anchors, as well as by the polarity and hydrophilicity characteristics of the solvent system. The specific conditions for poration or passive diffusion for placement of the SMMR in the skin are controllable factors. Using any combination of these factors, it is possible to control the localization of the dye within the skin layers and target cells.

In another embodiment, a small disposable film patch composed of polyolefin, polyester, or polyacrylate and having an SMMR dispersed into a transfer gel applied to the transfer side of the film patch, is used for SMMR application. The patch is applied with the gel side toward the skin and the gel contacts the external surface of the skin. Following the gel application, a poration or passive transfer technique is used to introduce the mixture into the appropriate skin layer(s) (as described above). Another embodiment of the SMMR application involves the use of a reservoir containing molecular tag or SMMR. This reservoir is used to either automatically or manually dispense a dose of the SMMR mixture topically prior to poration or passive transport. A non-limiting example of a topical dose is a small dot or spot from 100 μm to 5 mm. A smaller area is preferred in most embodiments, but a larger area is also contemplated. For measurement of glucose, the SIR is placed in the keratinocytes at 30 μm to 50 μm and up to 175 μm so that placement is precisely in the specific layer of the epidermis (e.g., above the dermal papillae and within or above the stratum basale), within a comparatively homeostatic keratinocyte stratum. The molecular tag or SMMR penetrates into the skin for some period of time (depending upon molecular size and solvent mixture used) to allow activation following passive diffusion kinetics (i.e., mass transport). Once activated, the change in fluorescence response of the skin cells to changes of extracellular and intracellular glucose is monitored directly using an optical reader.

The dyes may be introduced into the skin by passive diffusion over a period of 24-48 hours, more preferably over a period of 2-6 hours, and most preferably in 10 seconds to 5 minutes. Contemplated diffusion times include periods less than 48 hours, 24 hours, 10 hours, 6 hours, 2 hours, 1 hour, 30 min, 15 min, 10 min, 5 min, 1 min, 30 sec, 10 sec or 1 sec. An active mechanism utilizing skin permeation, electroporation, or ultrasonic poration is another procedure for introducing SMMRs into the skin. Pulse lengths for poration technologies are provided below. An example of an ultrasonic poration device includes those manufactured by Sontra Medical Corporation, Cambridge Massachusetts. Sontra and other commercial manufacturers of devices useful for this application have previously described a method for sensing glucose directly in the interstitial fluid surrounding the skin cells by removing fluid or gaining access to removed fluid for analysis. See e.g., J A Tamada, M Lesho and M J Tierney, “Weekly Feature: Keeping Watch on Glucose—new monitors help fight the long-term complications of diabetes.” IEEE Spectrum Online, Jun. 10, 2003 at website: <http://www.spectrum.ieee.org/WEBONLY/publicfeature/apr02/glu.html>(last visited Jun. 26, 2003). The methods and compositions of the invention do not remove fluid but, rather, place small quantities of solution containing low concentrations of SMMR into the skin for direct reading of the SMMR fluorescence spectral characteristics as an indication of both epidermal skin and blood glucose levels.

Electroporation, or more preferably passive transport, is used to introduce the SMMR solution into the skin. Electroporation has been utilized for introducing chemotherapy treatments, for introduction of DNA into living cells and tissues, and broadly recommended for introducing materials into tissues for cosmetic or medical treatment applications. If poration schemes are used, the optimized settings for an electroporation device are achieved by commercially available or by a customizable device having settings that provide conditions as described within this invention. Commercial systems utilizing a square wave voltage pulse have been described within the literature, such as those available from Genetronics Biomedical Corporation, 11199 Sorrento Valley Road, San Diego, Calif. 92121. Such a small device can be inexpensively made to have one or more constant settings for the optimized conditions disclosed for this invention.

Electroporation uses a short pulse electrical field to alter cell membrane permeability. Micro-pores form in the membrane of skin cells allowing the introduction of various molecular size mixtures into the cells at an appropriate depth of penetration for this specific inventive application. When the electric field is discontinued, the cells return to normal and one or more SMMRs introduced into the cell using the technique remains at the cellular site specifically within the epidermal cell until either the dye is chemically degraded and disposed of within the tissue or is sloughed off in a normal desquamating cycle. The process of sloughing off (or desquamating) follows a normal ten-day to twenty-day (typically fourteen-day) cycle as the residence time of epidermal keratinocytes moving from the basal layer (stratum basale) to the desquamating layer of the stratum corneum.

When employed, electroporation is optimized for use in this invention by selection of voltage range (from about 40 to 90 Volts), gap distance (from about 0 to 2 mm), pulse length (from about 150 to 250 ms), number or pulses (from about 1-10), pulse interval (from about 5 to 60 s), specific electrode design, and desired field strength (from about 40 to 60 V/cm). In addition, the selection of molecular tag molecules, solvent molecules, concentration, and lag times relative to measurement onset is determined as precisely as possible. In certain embodiments, specific parameters are determined empirically using specific solvent and SMMR selection. For example, optimization of electroporation involves the following specifications:

1. Output voltage range: 0 to +200 VDC;

2. Discharge capacitor (Cdis) values in microfarads are on or about: 200, 500, 700, 1000, 1200, 1500, 1700 μF;

3. Pulse type: exponential decay;

4. Pulse RtCdis decay time constant where Rt (total)=5+Rskin in parallel with 50 ohms. If Rskin>>50 ohms then Rt=55 ohms and Rt×Cdis=11, 27.5, 38.5, 55, 66, 82.5, 93.5 milliseconds (ms).

An ability to incorporate molecular tags into the skin without the use of external devices is a preferred embodiment due to the reduced cost and increased user convenience.

The present invention introduces one or more SMMRs into the skin and then measures the fluorescence of the SMMR (or SMMRs) as an indicator of the skin glucose concentration. This specific use of electroporation to introduce SMMRs into a specific skin site for measurement of SMMRs to report glucose has not previously been used. Electroporation or passive transport via diffusion and wicking is used explicitly to introduce one or more specific molecular compounds (SMMRs) and a solvent system into the appropriate skin layer. This invention goes beyond the use of electroporation, or an alternative passive molecule delivery, to treat or condition the skin, or to introduce medication. Rather, electroporation is used to more rapidly introduce a SMMR for subsequent fluorometric analysis.

In a preferred embodiment, the passive transdermal delivery solvent system employed is efficacious and safe. A more elaborate solvent regime may be applied than for the active mechanisms of traditional tattooing procedures, where dyes and inks are placed into the dermis for permanent marking; or poration schemes such as electroporation, laser-poration, iontophoresis, mechanical-poration, pressurized delivery and ultrasonic poration.

The more advanced solvent systems useful for passive transdermal delivery include, but are not limited to, e.g., creams, emulsions (both oil-in-water and water-in-oil), oils (ointments), gel film patches, a reservoir device, paints, polar solvents and non-polar solvents. Non-polar solvents are preferred, as these are most miscible with the SMMRs of the invention and the stratum corneum lipids cementing the keratinocyte lamellae in place. “Lipid solvent systems” have been reported in the literature for use in transdermal drug delivery, and are composed to resemble the chemistry of stratum corneum lipids. Such a mixture may also be used to place the SMMR into the appropriate point within the epidermis. Such a suggested mixture includes: (w/w): ceramide (50%), cholesterol (28%), palmitic acid (17%) and cholesteryl sulfate (5%). See, e.g., Downing D T, Abraham W, Wegner B K, Wilhman K W, Marshall J L: Partition of dodecyl sulfate into stratum corneum lipid liposomes. Arch. Dermatol. Res. 1993, 285:151-157.

The objective of these solvent systems is to provide passive transdermal SMMR delivery into the skin at a preferred depth of from about 10 to 175 μm (microns), more preferred from about 20 to 100 microns, and most preferred from about 20 to 50 microns. For example, the following solvents as additives to the final SMMR mixtures are added to the skin to initiate passive transport of the SMMR to the target cellular site. The materials listed aid the process of skin penetration for SMMRs and create a diffusion rate enhancing solvent system for transdermal delivery: dimethyl sulfoxide, ethanol, isopropanol, chloroform, acetic acid, saturated hydrocarbon solvent (with from 10 to 40 carbons as linear or branched chained molecules), soybean oil, hazelnut oil, jojoba oil, sweet almond oil, olive oil, calendula oil, apricot kernel oil, grapeseed oil, wheat germ oil, refined light mineral oil and mineral oil spirits, triundecanoin (akomed C), undecanoic acid, caprylic/capric glycerides (akoline MCM), caprylic/capric triglycerides, propylene glycoldiester of caprylic-/capric acid, and emu oil. All are low viscosity mixtures, preferably less than 35 cSt at 35° C. In certain embodiments, mixtures of one or more of the above oils are used in combination with a non-polar dilution solvent.

Factors that control the depth of penetration of the SMMR and its compartmentalization into the cells and domains of the epidermis include the polarity and partition coefficient of the SMMR as well as the solvent and the molecular size. The SMMR compound may also be derivatized so that it is readily taken up by the cell and then acted upon by enzymes that chemically alter the SMMR to prevent it from leaking out of the cell. One advantage of this type of approach is that the SMMR is only taken up in its active form by viable cells. Predictive schemes for determining appropriate derivatization of SMMR compounds are provided below. Alternative methods of derivatization well known to those skilled in the art are also contemplated as part of the invention.

The physical properties of the solvent system that strongly influence permeability in the skin include the molecular size, the vapor pressure, the water solubility, and the octanol water coefficient. Smaller molecular size increases the diffusion coefficient. The vapor pressure controls the balance between diffusion into the skin and evaporation from the surface. The water solubility and the octanol water partition coefficient determine the miscibility of the SMMR solution between aqueous interstitial fluid and hydrophobic core of the cell membrane.

For a passive solvent delivery system, the depth of penetration of the SMMR is strongly dependent on the volume of solvent added. Typically, the volume of SMMR used is from 10 μL to less than about 100 μL. Preferably, the concentration of SMMR is from 10 to 500 μM, more preferably from 100 to 300 μM, and most preferably from 150 to 250 μM. Target cells are exposed to extracellular concentrations in the range of 1 to 10 μM. Dilution of the SMMR concentration arises because of the diffusion properties from the surface of the tissue to the target cell site.

The proposed volume range added to the skin or other tissue is preferably from 1 to 50 μL, more preferably from 5 to 20 μL, and most preferably from 5 to 15 μL. Alternatively, a gel patch is used containing an SMMR coated surface of approximately 6 mm in diameter consisting of a concentration of SMMR preferably from 10 to 500 μM, more preferably from 100 to 300 μM, and most preferably from 150 to 250 μM.

Solvent systems used for SMMRs may be adjusted depending upon their molecular properties and compatibility with the specific SMMRs being delivered. For example, solvent hydrophobicity and polarity are noted along with the solubility properties of the SMMR, which will all have an effect on the movement of the SMMR into the tissue. Each SMMR has a certain affinity for the solvent and the tissue. The solvent's activity for delivering the SMMR directly to target tissue is a matter for empirical testing. One preferred embodiment of the invention uses an SMMR dissolved in DMSO (dimethyl sulfoxide) and further diluted in a saturated hydrocarbon solvent (with from 10 to 40 carbons as linear or branched chained molecules), or an alcohol (with from 2 to 4 carbons) at a volume ratio of 5:95 to 20:80, respectively. The optimum volume of DMSO in the delivery solvent is less than 20 percent, as the DMSO is used to facilitate dissolution of the SMMR into the carrier hydrocarbon mixture. The mixture is added to the tissue in the concentrations and volumes described above.

A gel patch may be used to apply the SMMR. In one embodiment, a gel is comprised of the SMMR in a volatile hydrocarbon solvent in suspension with a polymer such as PVA (polyvinyl alcohol). When placed against the skin or other living tissue, the heat of the skin causes the SMMR (dissolved in the PVA-hydrocarbon solvent) to diffuse into the skin. The final diffusion depth is controlled by length of application time. Volumes below 100 μL minimize extraneous transdermal delivery and maximize delivery into the epidermis target area. In some embodiments, optimum passive solvent delivery is attained by using a solvent mixture or emulsion that facilitates the movement of SMMR across the stratum corneum into the epidermis, but then dissipates rapidly to limit movement of the SMMR away from the target area. Solvent systems that have the lowest toxicity include water, saturated hydrocarbon oils, polyethylene glycols and glycerol. Solvents systems that include alcohols and dimethyl sulfoxide are less favored in this application since these solvents are less biologically inert.

In an exemplary embodiment, the SMMRs are applied directly to the surface of the skin and then passively allowed to penetrate the skin for a period of 1 minute to 5 hours, more preferably less than 4 hours, and most preferably less than 1 hour. Ideally, a solvent delivery system would be developed to provide SMMR delivery to the target tissue in less than 1 hour, more preferably less than 30 minutes, and most preferably in less than 5 minutes. This time period allows the passive diffusion of the SMMR into the appropriate epidermal cells.

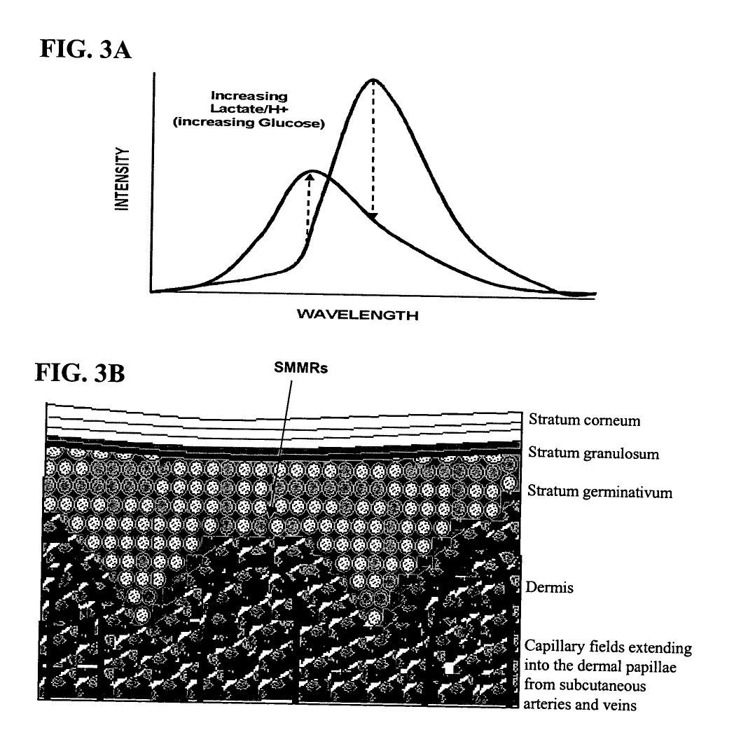

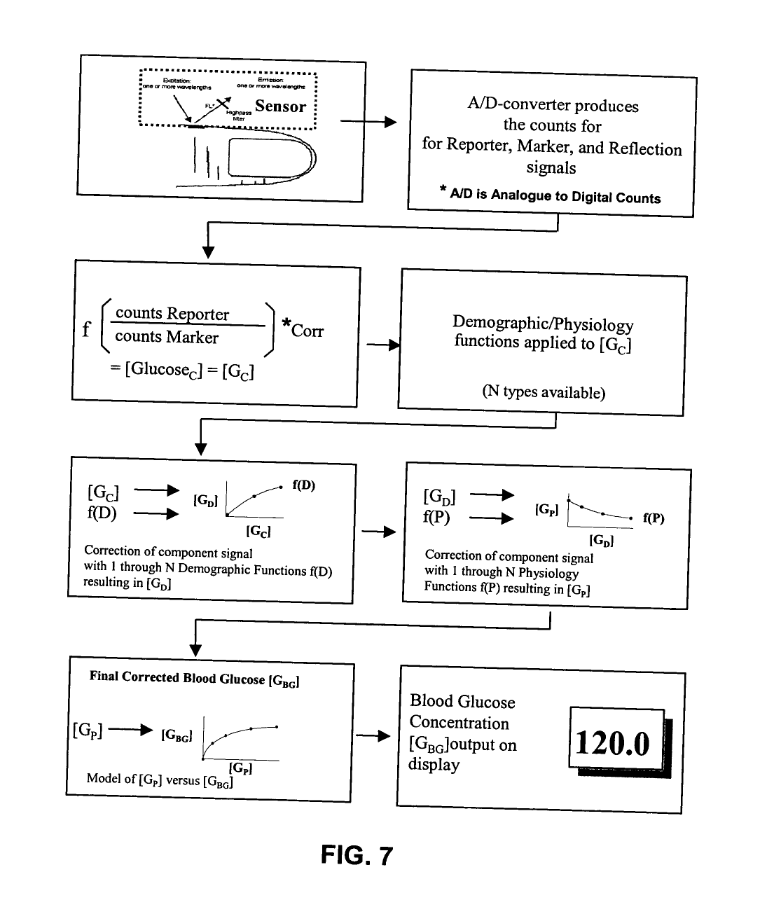

Once the one or more SMMRs are activated due to their placement within the skin, measuring fluorescence monitors the response of the skin cells to glucose. As described herein, the fluorescence mechanism used is either a direct or indirect indication of the glucose concentration in the target cell environment. Fluorescence is typically measured using an optical reader. In an exemplary embodiment, the optical reader calculates the skin response to glucose, applies first principles mathematical models to the response (as described below and shown in FIG. 7), and provides a determination of the blood glucose levels (See FIGS. 3, 8-9). The choice of the particular commercially available or custom designed optical reader that is compatible for use with the methods and compositions of this invention is within the ability of one skilled in the art of the invention.

FIG. 7 is a flow chart showing signal processing logic for determining final corrected blood glucose levels from a fluorescence measurement of an SMMR in the skin or other tissue. The Detector signal (as fluorescence or diffuse reflectance) is pre-amplified and the initial fluorescence ratio calculation is made. The signal is corrected using a diffuse reflection or empirical correction scheme (*Corr.) to produce G_{C}. Next, one or more of a series of Demographic functions are applied to the initial glucose (G_{C}) calculation to obtain a demographically corrected glucose level (G_{D}). This correction takes into account optical and minor physiological differences derived for demographic groups. The G_{D} value is then corrected for physiological differences such as exercise level, diabetes state, and health conditions resulting in a glucose value corrected for unique physiological conditions (G_{P}). The G_{P} value is subjected to a final correction for the lag component of translating diffusion of blood glucose to skin glucose levels resulting in the final blood glucose estimation (G_{BG}). This result is reported as the physiological blood glucose for an individual and is considered the analytical result.

Simultaneously, a quality value is calculated telling the user the quality of the measurement taken and of the resultant glucose value reported. Based on this quality value, the user may be instructed to make one or more additional measurements until the quality value is indicative of an accurately reported glucose result.

An obvious extension of this embodiment is the addition of SMMR molecules that are allowed to penetrate more deeply into the skin. In some embodiments SMMRs penetrate as far as the papillary layer of the dermis (upper corium), and into the reticular layer of the dermis (lower corium). In other embodiments, SMMRs are applied into the subcutaneous layers of the skin. In further embodiments, injection or ingestion of reporter molecules into the bloodstream, or into specific organs or tissues, is utilized. The resultant fluorescence response is measured at the site of application, e.g., by using an optical reader with remote optics (i.e., optical waveguides).

In alternative embodiments, the tissue being monitored is exposed, as in surgery or injury, or viewed remotely using invasive fiber optics, light pipes, or camera-based remote optics.

In alternative embodiments, the SMMR is applied directly to the tissue of a subject or is integrated into paint or gel. The paint or gel containing the SMMR is placed directly on the stratum corneum (outer skin) at one or more of several recommended sites. These sites include, but are not restricted to, e.g., the fingertip, the volar forearm, the upper arm, the foot, or any location where easy access to the skin is obtained without the need to disrobe. An added advantage for such a choice of location is to not induce embarrassment when in public display. A preferable location is on the side of the finger, where SMMRs are tagged just above the first knuckle. This avoids both inconvenience to the user and contamination brought about by prevalent use of the fingertip for routine activities. The volar forearm may also be used but is less preferred due to a decrease in vascularization at this skin site. The fingertip area is most preferred due to its increased relative vascularization as compared to the volar forearm and due to its convenience as a personal monitor site for both public and private use.

SMMRs may be packaged and sold in any clinically appropriate manner known to those skilled in the art, including in individual containers or in kits, and with or without disposable or non-disposable applicators. In an exemplary embodiment, a disposable applicator containing a solvent mixture, including but not limited, e.g., to a liquid or gel, and containing one or more SMMRs, is placed directly onto the outer skin and allowed to remain in place for a period of time. The time required for the SMMR to become activated is typically from 1 sec to 3 hours, preferably less than 2 hours, less than 1 hour, less than 30 min, less than 10 min or less than 5 minutes.

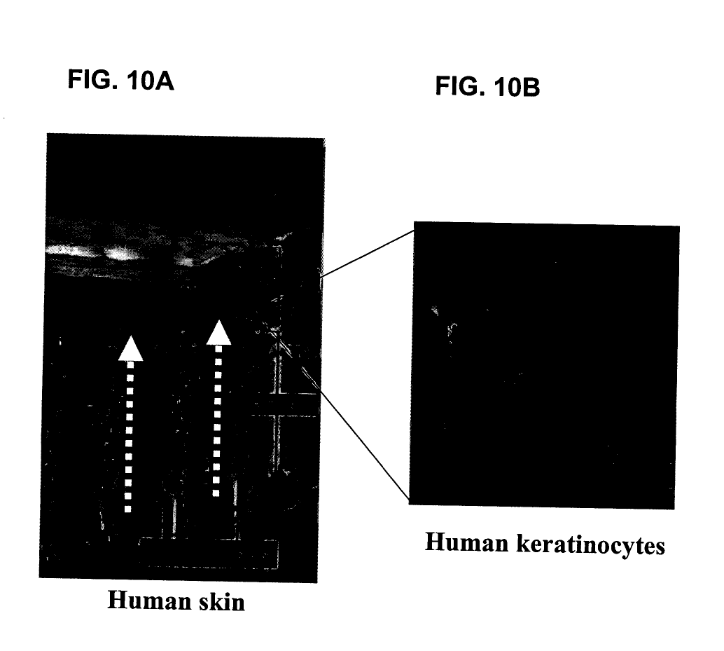

The mechanism of action of the SMMR is to act as an in vivo fluorescence reporter for metabolites that are stoichiometrically proportional to other non-fluorescent metabolites that are part of well characterized metabolic pathways (such as glycolysis). These stoichiometric relationships are most applicable when operative in living systems. These mechanisms and the techniques used to target these pathways are summarized in Scheme 1, FIGS. 10-17, and Table 1.

Mitochondrial Membrane Redox Potential

Once introduced to the epidermal intercellular fluid and keratinocytes, the SMMR will migrate preferentially to the target cells and cellular structures of the live epidermal cells (keratinocytes), which are directly above the basal layer (stratum basale). For effective measurement of glucose, the placement of the SMMR is preferably below the stratum corneum yet above the dermis, more specifically above the dermal papillae. This is because the device measures the glycolytic/metabolic activity of living cells, and the cells in the stratum corneum are essentially dead. The use of dyes that require activation by metabolic processes within the cell limits background interferences from a SMMR that has penetrated into dead tissue. Therefore, the methods and compositions of this invention are also useful for distinguishing between live and dead tissue using the principle of activation by metabolic processes. For example, SMMRs used as metabolic reporters will report the level of metabolic activity in target cells whether dead, normal, hypo-metabolic, or hyper-metabolic. This is due to the surprising discovery that SMMR-treated tissue provides unique spectral responses for metabolically active living tissue that are significantly different from the spectral responses from dead tissue. SMMRs that provide the most useful spectral responses include, but are not limited to, molecules providing fluorescence reporting of reducing equivalents, reduction-oxidation potential, and the presence of metabolites actively produced during biosynthetic processes such as glycolysis.

The SMMRs selected for use in the methods and compositions of the invention should have an affinity for the keratinocyte target cells and cellular structures located within the stratum spinosum. In some embodiments, the SMMR remains in place for several hours and/or throughout the life cycle of the epidermal keratinocyte cells and is eventually sloughed off as part of the desquamating layer of the stratum corneum. Epidermal keratinocytes have an average lifespan of 14 days, ranging from 4 to 20 days, and even up to 30 days, depending upon the individual subject skin health conditions, physical abrasion, or unprotected use of caustic or acidic chemicals on the skin. In most embodiments, the SMMR is introduced in low concentration, typically from 10 μM to 500 μM, at nominal volumes from 200 μL to 5 μL, respectively. The insertion of the SMMR within the epidermis provides an added safety feature, such that only short-term exposure to the SMMR occurs at any potential measurement site. In certain embodiments, the final interstitial fluid concentration of SMMRs used is from 0.01 to 500 μg·ml⁻¹, more preferably 1 to 100 μg·ml⁻¹, and most preferably 5 to 20 μg·ml⁻¹, based upon a molecular weight of approximately 380 Daltons for the SMMR. These concentrations apply irrespective of the molecular weights of the SMMRs, which range from approximately 100 Da to approximately 250 kDa. For the invention, the SMMRs for these molecular weight ranges can be placed into target tissues for reporting of glycolytic activity or other metabolic processes. Dosage of the SMMR solution involves adding 1 to 100 μL to a spot that is 0.1-5 mm in diameter, preferably less than 2 mm in diameter. One skilled in the art will be able to modify these dosage requirements based on empirical test results for specific metabolic reporting applications and signal intensity requirements.

SMMRs—Properties and Mechanisms

The methods and compositions of the present invention uses SMMRs such that two basic techniques are used for obtaining ratiometric measurements of glucose concentration or utilization versus fluorescence response. Mechanism 1 utilizes a combination of a reporter dye having a specific and fluorescence response proportional to a change in metabolite concentration, where that metabolite has a direct stoichiometric relationship to a change in glucose concentration. Mechanism 1 also utilizes a marker dye, which is stable but unresponsive to changes in glucose and is used explicitly to produce a reference signal. An example of a suitable marker dyes includes the class of coumarins, which fluoresce in the blue region of the spectrum and locate in the cytosol of the cell, but do not respond to a change in glucose or metabolite concentration. For embodiments where the reporter dye is located in the cytosol of the cell, it is necessary to have the marker in a different cellular compartment. One skilled in the art of photochemistry (including synthetic organic chemistry) can readily synthesize derivatives of these dyes that have these altered properties. For example, alkyl coumarins maintain the fluorescent properties of the coumarin parent but localize in the membranes of cell.

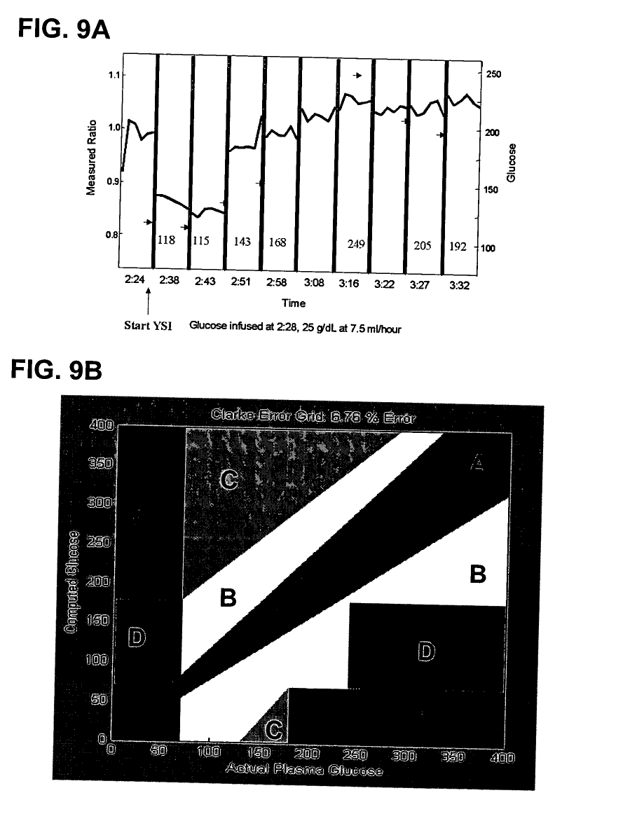

As an alternative, mechanism 2 utilizes a single dye having two wavelengths where fluorescence signal varies with the introduction of D-glucose concentration to living cells. This phenomenon is illustrated in FIGS. 3-5, and 8 with analytical results demonstrated in FIG. 9. The mechanisms by which SMMRs report on the rate and quantity of metabolic activity, particularly glycolytic processes for the invention are described herein.

Energy Transfer

A measurement of the change in fluorescence signal brought about by using an SMMR in vivo to track the formation of NAD(P)H (nicotinamide adenine dinucleotide (phosphate), reduced form) for energy transfer, FAD⁺ (flavin adenine dinucleotide, oxidized form) for energy transfer, can be used as an indirect measurement of the quantity of glucose entering a cell.

Metabolite Reporter

Metabolites present in the cell, which are produced as the result of glycolysis can also be measured in vivo, using SMMRs. These metabolites include pyruvate, pH (as lactate/H+), and cation or other ion pumping mechanisms such as Ca²⁺-pumping rate, Mg²⁺-pumping rate, Na⁺-pumping rate, and K⁺-pumping rate. Individually or in combination, these metabolites measured in skin using the techniques taught herein give a complete picture of epidermal skin glycolytic metabolism, and an indirect measure of the quantity of glucose molecules entering the cells.

Reduction-Oxidation Electric Potential

The measurement of electric oxidation-reduction potential across cell membranes in vivo is an accurate indirect indicator of glucose quantities entering the cell to fuel glycolytic processes. SMMRs reporting on changes in membrane potential are attached to cell membranes including the inner and outer cell membranes, the nuclear membranes, as well as those of organelles, such as mitochondrial membranes. SMMRs, acting as vital mitochondrial membrane stains, bring about a fluorescence response to changes in membrane potential. Membrane potential measured in skin cells using the techniques taught herein give a complete picture of epidermal skin glycolytic metabolism.

Direct—Emission Intensity

Proteins acting as SMMRs and as described herein can be used in vivo for direct measurement of intracellular or extracellular glucose. Fluorescence emission intensity response is proportional to the glucose concentration within the cell or external to the cell in interstitial tissue fluid or blood.

Direct—Lifetime

Proteins acting as SMMRs and as described herein can be used in vivo for direct measurement of intracellular or extracellular glucose. Fluorescence lifetime intensity response is proportional to the glucose concentration within the cell or external to the cell in interstitial tissue fluid or blood.

SMMRs useful for illustrating this mechanism include pH:lacate/H⁺⁻indicating molecules where two or more wavelengths change directly in proportion to a change in pH:lacate/H⁺ concentration. A two-photon fluorescence lifetime imaging within the dead uppermost layers of the epidermis (i.e., the stratum corneum) has been described, where the fluorophores are introduced into the tissue to measure the pH gradient across human skin. See, e.g., Hanson et al. Hanson et al., 2002, Biophysical Journal 83: 1682-1690, incorporated herein by reference. However, the skin tissue was removed from the animal prior to analysis, such that their in vitro technique was performed on dying tissue.

The essential characteristic in identifying a member of the class of SMMR dyes includes those compounds that report fluorescence changes in proportion to changes in glucose concentration for in vivo measurements. These dyes may be discovered empirically by screening large numbers of compounds for signal efficacy, or they may be designed using a basic understanding of photochemistry. The spectroscopic properties of SMMRs useful for routine analysis using low-cost instruments include, but are not limited to, one or more of the following: molecules that exhibit a large molar absorption coefficient (10,000 L mol⁻¹ cm⁻¹ and above), molecules that exhibit a high Stokes shift (e.g., 20 to 150 nm), long (e.g., 2 hours to 4 weeks) residence time at target site, molecules that are highly photostable (e.g., less than 5 percent signal loss at use excitation power), molecules that exhibit little or no excited state chemistry (i.e., inert or non- reactive in excited state), and molecules that exhibit large fluorescence quantum yield (e.g., Quantum Yield [φ_{F}] greater than 0.4).

Examples of suitable SMMRs include, but are not limited to, modifications of fluorescence dyes to include molecular size attachments as: acetoxy methyl esters, chloro-methyl derivatives, alkyl chain adducts, highly charged moieties, enzyme substrate mimic, enzyme cofactor tethers, and membrane binding tethers. The basic starting dyes used to develop SMMRs are polycyclic aromatic hydrocarbon dyes, including, but not limited to: rhodamine 123; di-4-ANEPPS, di-8-ANEPPS; DiBAC₄(3); RH421; tetramethylrhodamine ethyl ester, perchlorate; tetramethylrhodamine methyl ester, perchlorate; 2-(4-(dimethylamino)styryl)-N-ethylpyridinium iodide; 3,3′-dihexyloxacarbocyanine, 5,5′,6,6′-tetrachloro-1,1′,3,3′-tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene dyes especially 2′,7′-bis-(2-carboxyethyl)-5-(and-6)-carboxyfluorescein; and benzenedicarboxylic acid, 2(or 4)-[10-(dimethylamino)-3-oxo-3-H-benzo[c]xanthene-7-yl]; and iodine dissolved in potassium iodide. Other dyes or stains that are useful as SMMRs include, but are not limited to: fluorescein derivatives, modified coumarin; derivatives of coumarin, anthraquinones; cyanine dyes, azo dyes; xanthene dyes; arylmethine dyes; pyrene derivatives; and ruthenium bipyridyl complexes. An exemplary backbone of a contemplated SMMR is shown in FIG. 17C and described further in the Examples, infra.

However, the use of other dyes exhibiting similar characteristics and chemical structure for the in vivo determination of glucose is an extension of this concept, and an important aspect of this invention. Dyes that exhibit the provided characteristics and chemical structures would be known to one skilled in the art. Alternative embodiments would differ only in the dye selected and in the optimization of the techniques shown herein. This invention includes methodologies to develop optimum conditions for the use of other dyes expressly for the purpose of extending or refining this application.

One specific redox potential indicating dye, namely Rhodamine 123 (Rh123), provides an illustrated working example of the present invention. Rh123 dye has the systematic name (2-(6-Amino-3-imino-3H-xanthen-9-yl) benzoic acid methyl ester), given CAS No. 62669-70-9. Membrane reporting redox potential indicating dyes such as Rh123 have been used in concentrations of 10-150 μM for multiple applications, many related to intracellular mitochondrial activity, specifically for measurement of fluorescence response proportional to changes in transmembrane redox potential for the expressed purpose of research in the mechanics of cell metabolism.

Rh123 is commonly known as green fluorescent mitochondrial dye and is widely applied in cytometry studies involving mitochondrial membrane potential. Its spectral properties include an excitation maximum wavelength of 485 to 505 nm with an emission wavelength of 525 to 534 nm. It exhibits an absorption maximum from 485 to 505 nm and has a molar absorption coefficient of 97,000 Lmol⁻¹ cm⁻¹. This dye is an orange-red solid that is soluble in methanol (MeOH), dimethyl sulfoxide (DMSO) and dimethylformamide (DMF). These dyes are light sensitive. Once in solution they should be kept at less than 5° C. and protected from direct illumination for long-term storage and optimum efficacy. Rh123 has a molecular formula of C₂₁H₁₇ClN₂O₃ and a molecular weight of 381 daltons. The molecule has low toxicity and has a reported intravenous lethal dose for animals (LD10) of approximately 20 mg/kg of body weight; and a fifty percent lethality (LD50) of 89.5 mg/kg (i.v.) in rats (Merck).

Rh123, and other dyes exhibiting similar molecular structures, have a specific set of chemical properties whereby the molecule is fluorescent, cationic (i.e., positively charged), of low molecular weight, lipophilic, and configurable as a water-soluble salt. Having these molecular properties, dyes such as Rh123 exhibit preferential binding to negatively charged mitochondrial membrane lipids. The final quantity of dye, which collects within the mitochondrial membrane, is dependent on the molar concentration of the dye within the surrounding medium (i.e., intercellular and cytosol concentrations) and, more importantly, the mitochondrial membrane potential. The dye is distributed into the membrane by means of general diffusion such that the molecules move into the cell and then to the mitochondrial membrane at a rate that is dependent on chemical kinetics and metabolic rate. Thus, increases in temperature and thereby metabolic rate, will increase the rate of random motion that is driving the concentration of Rh123 molecules in solution to equilibrium.

Accurate measurements can be made over nominal temperature ranges from 75 to 105° F. or wider. Variations in the subject temperature wider than approximately ±5° F. of the target tissue require re-calibration, as noted elsewhere. The method used to recalibrate for any temperature range is to make certain that the temperature is measured while the calibration is performed using equations 1-5 and 13-16 of the invention. Any subsequent measurement of the test sample may be performed within ±5° F. without concern for temperature variation. Each cationic molecule of dye accumulates stoichiometrically as negatively charged moieties within the inner mitochondrial membrane of healthy metabolizing cells at a concentration dependent rate. The final concentration of dye uptake for each cell is dependent upon the number of mitochondria present within the treated cells as well as the changes in the mitochondrial membrane potential within each cell.

Under conditions where glucose is the major metabolic substrate for the cell, oxidative phosphorylation is fueled by the products of glycolysis. See, e.g., Johnson, L. V., et al. J. Cell Biol. 83, 526 (1981). Additional discussions describing research applications of membrane potential-indicating dyes are found in, e.g., R. C. Scaduto, and L. W. Grotyohann, Biophysical Journal 76, 469 (1999) and related references. For most of the reducing reactions that occur in cells, the reducing power is provided by NAD(P)H. The pH gradient that generates the mitochondrial membrane potential is fueled by NADH. This NADH may be derived from the Krebs citric acid cycle as well as from glycolysis.

Illumination using energy at approximately 490 nm excites Rh123 directly and its fluorescence emission can be detected at approximately 530 nm. The final baseline intensity of the dye is proportional to the concentration of dye present at the mitochondria, and to the mitochondrial density. The changes in the net fluorescence intensity of Rh123-like dyes are directly proportional to the changes in the membrane potential of the cell. Molecules behaving in this manner are referred to as transmembrane redox potential indicating SMMRs (FIG. 14). Metabolic activity fueled by glucose is indicated by Rh123-like dye fluorescence intensity.

Specific chemical agents are known to disrupt oxidative phosphorylation and glucose metabolism. Any such agent causing decreased cellular respiration, cellular energy balance, and cell viability will affect the fluorescence intensity of the dye bound to the mitochondria. A decrease in the glucose concentration available to the cell causes a reduction in ATP production due to depletion in metabolism from lowered oxidative phosphorylation. Such a decrease in glucose concentration is indicated by a corresponding decrease in fluorescent intensity. The demonstration of a linear increase in mitochondrial-bound Rh123 fluorescence with changes in respective glucose concentration for immortal cell lines has been shown. See, e.g. Borth, et al. Cytochemistry 14, 70 (1993), incorporated herein by reference. Borth demonstrated that for isolated 3D6-LC4 human-mouse heterohybridoma cells in suspension the mean fluorescence intensity was dependent upon glucose availability (i.e., concentration) rather than to either increased growth rate or metabolic rate.

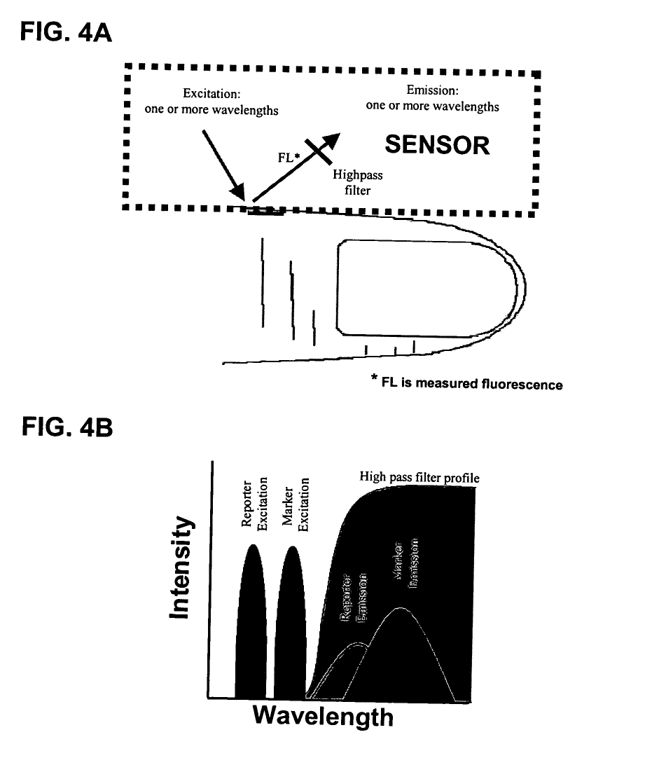

One embodiment of the invention utilizing a sensor that acts as a reporter and marker channel detector is provided in FIG. 4. The signal detected by the sensor is derived from the relaxation of a metastable excited state generated by the absorption of energy from a lamp, light-emitting diode, or laser source. The fluorescence process is repetitive, meaning the fluorescence response to glucose can be measured repetitively or continuously, as long as the molecular tag molecules are not destroyed or removed. The same molecular tag can be repeatedly excited and its emitted energy detected. In the methods and compositions of this invention, the emission intensity, given from a known concentration of at least one molecular tag, is proportional to the number of energy transfer events from NAD(P)H to Rh-123 where Rh-123 acts as the SMMR. Compounds exhibiting such a mechanism of action are representative of a family of compounds referred to as “energy transfer from reducing equivalents indicating SMMRs” (FIG. 12). The number of these energy transfer events is proportional to the rate of glycolysis modulated by the glucose concentration of the intercellular environment and ultimately to the blood glucose concentration.

The chemical structure of Rh123 is shown below as Structure A. This dye belongs to a broad range of compounds referred to as xanthene dyes. The general structure of xanthene dyes is shown in Structure B. Substitution of these dyes at any of the positions marked “R” on the xanthene moiety influences the wavelengths of absorption and emission while substitution of the phenyl ring at position 9, shown in Structure B, influences the solubility of the molecule. As drawn, the molecule absorbs light in the ultraviolet region of the spectrum. Substitution at the positions marked R with a heteroatom that readily exchanges hydrogen causes extended conjugation across the ring, wherein the molecule absorbs in the visible region of the spectrum. In the case of Rh123, the heteroatom is nitrogen and the R group may exist as an amino group or an imino group. Many xanthene dyes are amphipathic, that is, they have both polar and non-polar regions on the molecule. This property gives the molecule a high affinity for binding to the surface of biological membranes.

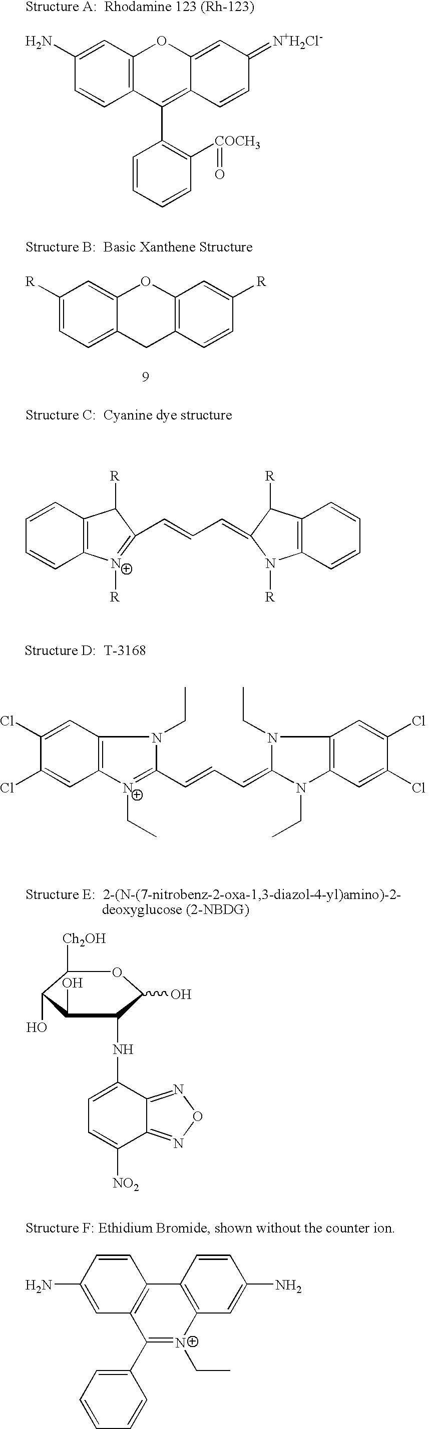

Molecular structures of some of the SMMRs of the invention include those shown as Structures A-F. Other mitochondrial or membrane potential dyes useful for this invention include any molecules exhibiting properties as defined for Rh123 above (Structure A) including those mentioned here, and general compounds falling within these molecular structures, activity, solubility, toxicity, and overall action as described. Specific dyes meeting some or all of these requirements include, but are not limited to, the following.

Xanthene Type Dyes (Structure B):

Examples of Xanthene type dyes include: TMRE as tetramethylrhodamine ethyl ester, perchlorate (C₂₆H₂₇ClIN₂O₇. Molecular Weight 515), TMRM as tetramethylrhodamine methyl ester, perchlorate (C₂₅H₂₅ClIN₂O₇. Molecular Weight 501), Dihydrorhodamine 123 (C₂₀H₁₈N₂O₃. Mwt: 346), Dihydrorhodamine 123, dihydrochloride salt (C₂₀H₂₀Cl₂N₂O₃. Mwt: 419).

Cyanine Type Dyes (Structure C):

Examples of cyanine type dyes include: 5,5′,6,6′-tetrachloro-1,1′,3,3′-tetraethyl-benzimidazolylcarbocyanine both the chloride and the iodide salts.

Bis-Oxonol Dyes

Examples of bis-oxonol type dyes include: DiBAC4(3) as bis-(1,3-dibarbituric acid)-trimethine oxanol (C₂₇H₃₉N₄O₆, Molecular Weight 519).

Styryl Pyridinium Dyes:

Examples of styryl pyridinium type dyes include: RH421, N-(4-sulfobutyl)-4-(4-(p-(dipentylamino)phenyl)butadienyl)-pyridinium (C₂₉H₄₂N₂O₃S, Molecular Weight 498.72), DASPEI as 2-(4-(dimethylamino)styryl)-N-ethylpyridinium iodide) with a molecular formula of C₁₇H₂₁IN₂ and molecular weight of 380 daltons, Pyridinium, 4-(2-(6-(dibutylamino)-2-naphthalenyl) ethenyl)-1-(3-sulfopropyl)-, hydroxide Di-4-ANEPPS (C₂₈H₃₆N₂O₃S; Molecular Weight 481).

Carbocyanine Dyes (Structure D):

T-3168 is a cationic carbocyanine dye that yields green fluorescence. It accumulates in mitochondria and is a sensitive marker for mitochondrial membrane potential. It exists as a monomer at low concentrations and forms J-aggregates at higher concentrations that exhibit a broad excitation spectrum and an emission maximum at ˜590 nm.

Glucose Analog (Structure E):

2-(N-(7-nitrobenz-2-oxa-1,3-diazol-4-yl)amino)-2-deoxyglucose (2-NBDG). The 2-NBDG fluorophore typically displays excitation and emission maxima at around 465 nm and around 540 nm, respectively. It is visualizable using optical filters designed for fluorescein and is sensitive to its environment. A fluorescent nonhydrolyzable glucose analog 6-NBD-deoxyglucose (6-NBDG) is also available commercially to track glucose diffusion rates in cells. (Molecular Probes cat. no. N-23106).

Viability and Toxicity Dyes (Structure F):

The cell-impermeant Ethidium Bromide is excited by an argon-ion laser and is useful for detecting and sorting dead cells by flow cytometry. It is also used in combination with fluorescein-based probes (such as calcein, CellTracker Green CMFDA or BCECF) for two-color applications, and as a marker when a reporter dye responds at only one emission wavelength.

The dyes mentioned above are available commercially in relatively pure forms from suppliers of custom molecules as well as from Biotium, Inc., 3423 Investment Blvd. Suite 8, Hayward, Calif. 94545. The preceding dyes are commonly described in the scientific literature as molecules “that stain mitochondria in living cells in a membrane potential-dependent fashion [with varying excitation and emission wavelengths].” See the Merck Index (The Merck Index: An Encyclopedia of Chemicals, Drugs, and Biologicals, Thirteenth Edition, Maryadele J. O'Neil, Ann Smith, Patricia E. Heckelman, John R. Obenchain Jr., Eds., Merck & Co., Inc., Whitehouse Station, N.J., USA, 2001), and other comprehensive collections of properties for organic compounds. Such references provide information regarding details of chemical and physical properties of molecules, including availability, solubility, and synthesis for each class of molecule described herein. Additional information is available from commercial suppliers, e.g., Aldrich Chemical Company, Inc., 1001 West St. Paul Avenue, Milwaukee, Wis. 53233; Sigma Chemical Co., Inc., 3050 Spruce Street, St. Louis, Mo. 63103; Fluka-RdH, P.O. Box 2060, Milwaukee, Wis. 53201 USA; Molecular Probes, Inc., 29851 Willow Creek Rd., Eugene, Oreg. 97402 USA; and other manufacturers. Preferred dyes, acting as SMMRs, emit fluorescence signals at wavelengths above 450 nm.

The design of specific SMMRs for particular locations and mechanisms within tissue takes into consideration the specific molecular properties of the SMMRs. Under conditions where intracellular and extracellular pH measurements are to be made simultaneously using redox potential measurements, for example, it is important that the dyes emit in different wavelength regions of the electromagnetic spectrum. The spectral regions that are preferentially selected for emission bands are bands at or about 100 nm wide and centered at or about 600 nm, 700 nm and 800 nm. These regions contain little or no autofluorescence and a relatively small and stable absorption background. These properties allow a relatively interference-free measurement for the SMMR fluorescence.

The use of known mitochondrial specific redox potential fluorescing dyes, or energy transfer fluorescing dyes for the purpose of sensing live human keratinocyte glucose metabolism in situ has not been previously described. The fluorescence intensity of mitochondrial-bound redox potential sensing dyes, as well as dyes reporting on reducing equivalents via energy transfer, are indicative of the reduction potential for the sum of oxidative phosphorylation, fatty acid metabolism, and NADH shuttling. The use of redox potential fluorescing dyes has not previously been applied in situ for direct determination of in vivo skin glucose concentration. The ability to use these dyes for such an application is based upon an understanding of skin glucose metabolism, the mechanism and importance of reducing equivalents in glycolytic metabolism of skin, an understanding of the fluorescent properties of the selected dyes, and an understanding of the optical properties of skin. In addition, these methods and compositions require a detailed understanding and optimization of dye introduction to the human keratinocyte cells; of the derivation of the appropriate conditions for temperature, pH, concentration, purity, lag times, reaction times, response times, quantum yields, optical properties of the various skin layers for the appropriate excitation and emission wavelengths, of the concentration/fluorescence response model, metabolic models for skin; and of the lag time between blood glucose and skin glucose concentrations.

In contrast, U.S. Ser. No. 60/438,837, which is herein incorporated by reference, describes a direct mechanism for in vivo fluorescence measurement of glucose. The direct measurement technologies utilize a mechanism for fluorescent spectra that responds directly to the glucose molecule itself, rather than ones that respond to changes in a related metabolite or analyte.

The invention will be further described in the following examples, which do not limit the scope of the invention described in the claims.

### EXAMPLE 1

Relating Fluorescence of Mitochondrial Membrane Probes to D-Glucose Concentration

Described herein is a technique for establishing the dose-response relationship for tracking skin and blood glucose concentrations using mitochondrial membrane potential. The SMMRs used in this embodiment have the demonstrated property of being mitochondrial-specific vital stains that respond in a direct relationship to the rate of glycolysis, which is directly related to intracellular glucose concentration. The fluorescence response of one specific embodiment of this invention uses SMMRs exhibiting an excitation wavelength of from 290 to 790 nm, more preferably 400 to 490 nm, and most preferably from 440 to 490 nm, i.e., the wavelengths used to excite a fluorescence response of the SMMR. The fluorescence is monitored at above 480 nm, preferably above 490 nm and most preferably at 501 nm. The upper range for monitoring is at or below 790 nm. Excitation and emission wavelengths are selected to minimize absorption and fluorescence by endogenous chromophores and fluorophores.

Mitochondrial activity as monitored by oxidative phosphorylation is directly correlated to the number of reducing equivalents derived from NADH, which is generated by aerobic glycolysis or from the conversion of pyruvate to CO₂ within the mitochondrial organelles. For aerobic metabolism the number of reducing equivalents is directly and quantitatively (i.e., stoichiometrically) equal to ten times the number of glucose molecules entering the metabolizing cell. The glucose glycolysis pathway is depicted in FIG. 18.

For anaerobic glycolysis, the metabolism of glucose to pyruvate generates two NADH molecules in the cytoplasm of the cell per glucose molecule. This NADH is available to the mitochondria by a NADH shuttle system in the mitochondrial membrane. The stoichiometry of this process is such that for every glucose molecule metabolized, two pyruvate molecules are generated. The conversion of pyruvate to acetyl CoA and subsequently to carbon dioxide in the Krebs citric acid cycle is accompanied by the generation of an additional four NADH molecules per pyruvate metabolized. Therefore, the overall yield of NADH per glucose metabolized is ten molecules. The final product of glucose metabolism is carbon dioxide and water.

Under conditions where the most important metabolic substrate is glucose to drive glycolysis, as occurs in the skin, the fluorescence response is linear and in direct proportion to the intracellular glucose concentration. Once the SMMR is introduced to the appropriate cell layers (specifically live epidermal cells (keratinocytes) directly above the basal layer (stratum basale)), the SMMR enters the keratinocyte cell membrane and accumulates in the cell mitochondria. When the SMMR is in place for living cells, the fluorescence response may be fully sufficient for in vivo noninvasive determination of the rate of oxidative phosphorylation (i.e., the Kreb's cycle) for living human epidermal cells.

The fluorescence response of these dyes is then related to blood glucose level by the relationships shown in equations 1 and 2. The action of any SMMR or other dye meeting the requirements outlined above include those molecules that are mitochondrion-selective vital SMMRs, which act to indicate the NAD(P)H activity within the mitochondria and, in some cases, the cytosol. The dyes, when used singly or in combination, have an affinity for the mitochondria and accumulate within this organelle in a quantity that is directly proportional to the living cell membrane potential. In other preferred embodiments, all such dyes useful for this invention are nontoxic, non-carcinogenic, non-teratogenic, and do not deleteriously affect the skin when exposed to ultraviolet light or natural sunlight. In preferred embodiments, such dyes included in the present invention are highly fluorescent, are evenly dispersible in the cell and interstitial cell fluid, cannot aggregate or agglomerate, and do not exhibit binding-dependent fluorescent efficiency and quantum yields. In most embodiments, these dyes do not inhibit or restrict normal cell metabolism nor adversely affect cell viability or health in the concentrations and manner used.

Indirect measurement of blood glucose concentration is made as follows. A two-dye measurement regime is provided wherein a non redox indicating dye, which exhibits stable fluorescence with a change in glucose or other metabolites (i.e., the marker dye); and a dye that exhibits direct changes in fluorescence intensity with a change in glucose (i.e., the reporter dye) are measured individually. Optimized dyes are safe, relatively permanent, and non-absorbing into the dermal tissue. The individual dye fluorescence intensity measurements are made using an ultraviolet or visible light emitting diode (LED) or laser diode for an excitation source. The emission detector (i.e., the sensor) collects the light from the emission of the dye signal within the skin. In most embodiments, the sensor device also calculates the ratio of reporter dye fluorescent (following a predetermined lag time as lagt) to the marker dye fluorescence (following the same lag period lagt). A linear univariate computational formula for calibrating such an analyzer for blood glucose is given in equation (1) as:

$$[ {Glucose}_{Blood} ] = {k}_{1} \times \frac{Reporter {Fluorescence}_{lagt}}{Marker {Fluorescence}_{lagt}} + {k}_{o} \qquad ( 1 )$$

where k₁ is the regression coefficient (slope for the line) describing a change in fluorescence for the Reporter to Marker ratio versus glucose concentration in the blood, and k₀ is the calibration line intercept. Additionally, a change in glucose concentration over a time interval from T₁ to T₂ involves the relationship given in equation (2) as:

$$\begin{matrix}Δ [ {Glucose}_{Blood} ] = {k}_{1} \times \\ \frac{Reporter {Fluorescence}_{lagt} ( {T}_{2} - {T}_{1} )}{Marker {Fluorescence}_{lagt} ( {T}_{2} - T1 )} + {k}_{o}\end{matrix} \qquad ( 2 )$$

where Δ[Glucose_{Blood}] represents the change in blood glucose concentration and the terms (T₂-T₁) represent the change in reporter or marker dye fluorescence over the time interval.

The dyes described within this invention may also exhibit an exponential, logarithmic, power, or other non-linear relationship between fluorescence intensity and glucose concentration such that the computational formula for calibrating such an analyzer for blood glucose using an exponential relationship is given in equation (3) as:

[Glucose_{blood}]=k₀ e^{k}₁^{R} (3)

where R is the ratio of Reporter Fluorescence_{lagt} to Marker Fluorescence_{lagt}.

The computational formula for calibrating such an analyzer for blood glucose using a logarithmic relationship is given in equation (4) as:

[Glucose_{blood}]=k₀+k₁ ln R (4)

where R is the ratio of Reporter Fluorescence_{lagt} to Marker Fluorescence_{lagt}.

The computational formula for calibrating such an analyzer for blood glucose using a power relationship is given in equation (5) as:

[Glucose_{blood}]=k₀x^{k}₁ (5)

where R is the ratio of Reporter Fluorescence_{lagt} to Marker Fluorescence_{lagt}.

R can represent the intensity at either a measure wavelength referenced to a baseline wavelength, or as described above as the ratio of Reporter Fluorescence_{lagt} to Marker Fluorescence_{lagt}.

The methods and compositions of the invention relate to the measurement of glucose using the mitochondrial membrane potential as the metabolic marker. However, as described in Scheme 1 (FIG. 17A), other pathways may also be used to make this measurement and/or to give additional or validation information about the measurement.

### EXAMPLE 2

Relating Fluorescence of Energy Transfer to a Reporter Dye to D-Glucose Concentration

SMMRs can also be used to report the metabolic state of cells, by using such dyes to monitor NAD(P)H concentration. NAD(P)H can be excited at wavelengths of 340 to 360 nm. Over this wavelength range, the molar absorption coefficient of SMMRs such as Rh123 is low (Rh123 ε<500 L·M⁻¹ cm⁻¹ from 345 nm to 425 nm compared with 6.3×10³ L·M⁻¹ cm⁻¹ for NADH. (NADH and NAD(P)H are indistinguishable by their absorption or emission spectra.) Excitation at 350 nm of tissue that has been incubated with Rh123 shows a distinct fluorescence signal at 530 nm. This fluorescence arises because of collisional energy transfer from NAD(P)H to the Rh123. Under conditions where the energy transfer is efficient this process leads to an enhancement of the sensitivity with which NAD(P)H can be detected, shown in equation (6) as:

$$NAD ( P ) {H}^{*} + Rh - 123 {\to }^{energy transfer} Rh - {123}^{*} + NAD ( P ) H \qquad ( 6 )$$

The excited state of Rh123 (Rh-123*) relaxes to the ground state by fluorescence with almost unit efficiency. As a result, the sensitivity of the fluorescence technique to monitor NAD(P)H is increased by at least an order of magnitude or more over autofluorescence. One skilled in the art of photochemistry can easily identify similar conjugated molecules to be used for collisional energy transfer reporting for reducing equivalent molecules, including predominantly NAD(P)H and FADH.

### EXAMPLE 3

Relating Fluorescence of Membrane Localizing Reporter Dyes to D-Glucose Concentration

Membrane localizing dyes are used to detect activity of membrane bound proteins. Dyes such as diphenylhexatriene have been used in the past to monitor membrane fluidity. However many dyes may be used to monitor membrane activity by energy transfer mechanisms. Dyes that are useful in this role include molecules that have lower singlet energy levels than amino acid residues such as tryptophan, that is, they absorb light at longer wavelengths than 320 nm. Suitable dyes include, but are not limited to xanthenes, cyanines as well as diphenyl hexatriene and its derivatives. The efficiency of energy transfer is determined by the separation of the donor and acceptor pair and is given by the expression in equation (7):

$$E = \frac{{R}_{o}^{6}}{{R}_{o}^{6} + {r}^{6}} \qquad ( 7 )$$

where E is the efficiency, Ro is the Förster radius and r is the donor acceptor separation. The Förster radius is defined as the donor acceptor separation that gives an energy transfer efficiency of 50% and is dependent on the donor and acceptor used. This mechanism is particularly useful for proteins that physically move during activity such as glucose transporter (GluT) proteins. The distance between excited state amino acid residues and an acceptor molecule, usually located in the membrane, will change as the protein carries out its function and hence the efficiency with which the acceptor fluoresces will vary with the activity of the protein. GluT undergoes conformational changes as it transports glucose across the membrane. Excitation of tryptophan residues in a GluT molecule leads to energy transfer to the membrane bound acceptor and the overall fluorescence is then dependent on the concentration of glucose transported across the membrane.

### EXAMPLE 4

Relating Fluorescence of pH Indicating Reporter Dyes to D-Glucose Concentration

Determination of the cytosolic intracellular pH relates the ratio of the cytosolic NAD/NADH ratio to the pyruvate/lactate ratio by the expression as can be derived from textbook information such as that provided by L. Stryer, Biochemistry, W. H. Freeman and Co., New York, 1988 (3^{rd} Ed.), pp. 363-364, Chapter 18. An example calculation of intracellular pH is given in equation (8):

$$\frac{{[ NAD ]}_{cyt}}{{[ NADH ]}_{cyt}} ∝ \frac{[ pyruvate ] \times {10}^{- p H}}{[ lactate ]} \qquad ( 8 )$$

The measurement of pH as a direct indicator of lactate/H⁺ concentration in skin yields direct information on skin and blood glucose concentrations. The parameter of pH as −log₁₀[H⁺] can be measured using calibrated pH sensitive dyes or with a variety of known microprobe electrodes specifically designed for pH determination. One embodiment involves a series of techniques that allow the placement of a specialized “tattoo”, or more precisely the “active viewing window” comprised of one of a choice of specific pH indicating SMMR, into the epidermis using methods including, but not limited to, electroporation, direct application by painting with specific transporter solvent mixtures, tattooing methodologies, laser poration, sonic poration, iontophoresis, mechanical-poration, solvent transport, wicking, pressurized delivery or by an equivalent active or passive application technique. In another embodiment of the SMMR application techniques a small disposable polymer patch comprised of an SMMR dispersed into a transfer gel is applied to the skin using a pre-specified protocol. Another embodiment is to have a small dispenser with a specialized tip for placing a measured dose of the SMMR, with or without a solvent mixture, onto the skin. The molecular tag or SMMR is allowed to penetrate the skin for some period of time to allow activation (from 1 minute to 3 hours, depending upon the mixture used). Once activated, the response of the skin cells to glucose is monitored directly using an optical reader on the SMMR-treated viewing window. The optical reader calculates the skin fluorescence response to glucose, applies first principles mathematical models to the response, and provides a determination of the blood glucose levels. The concepts and results are demonstrated in FIGS. 1-9, especially FIGS. 3-5, 8, 9. A quality value may be simultaneously calculated in the optical reader/sensor telling the user the quality of the glucose value reported. Based on this quality value, the user may be instructed to make one or more additional measurements until the quality value is indicative of an accurate result.

These features provide a technique for establishing the dose-response relationship for tracking glucose. See, e.g., FIGS. 3, 8, 9. Specific SMMRs to be used have demonstrated properties of being pH:lactate/H⁺-indicating SMMRs that respond in a direct linear, exponential, or sigmoid relationship to intracellular glucose concentration. An increase in intracellular glucose causes a direct increase in intracellular lactate/H⁺ via glycolysis, thereby decreasing the intracellular pH in real-time by a stoichiometric inverse proportionality, relative to the increase in glucose concentration. The visible light response of these SMMRs is such that a diffuse reflection or fluorescent emission spectrum or signal obtained after excitation at one or more optimum wavelength(s), e.g., between 300 nm and 750 nm, and more preferably at least 450 nm, is directly correlated to the quantity of glucose available to fuel metabolic (glycolytic) activity and is unaffected by cellular metabolic rate. Therefore the absorption/diffuse reflection or fluorescence spectrum measured is in direct proportion to the intracellular glucose concentration. The reaction velocity assumption set for quantitative analysis of metabolites, including pH, is described above.

When the SMMR is comprised of a mixture including a fluorescent indicator dye, then fluorescence spectroscopy may be used to determine the pH/lactate/H⁺ in the microenvironment of the cell. Once the tattoo or SMMR mark is produced, these methods and compositions can be used for in vivo noninvasive determination of the rate of glucose utilization, whether occurring by glycolysis, oxidative phosphorylation (i.e., the Kreb's cycle), or a combination of these metabolic processes. In the case of mammalian or human keratinocytes, anaerobic glycolysis is the pathway defined using this technique. The determination of glucose is accurate for living mammal or human epidermal cells as long as the SMMR remains within the area of the stratum spinosum. SMMRs meeting the requirements for this embodiment include but are not limited to phenolphthalein, which is useful for absorption measurements of pH. Fluorescent SMMRs include but are not limited to molecules that are xanthene dyes, especially 2′,7′-bis-(2-carboxyethyl)-5-(and-6)-carboxyfluorescein, (BCECF), and other standard pH indicating fluorescent dyes available from, e.g., Aldrich, Sigma, Molecular Probes, and other manufacturers. Alternatively, as the structures are known, those skilled in the art may be able to synthesize these materials.

Other SMMRs meeting the requirements of this invention include BCECF, which can be used at 439 nm and 490 nm excitation. pH is calculated from the emission detected at 520 nm. Measurements may also be made of the lifetime of BCECF, and such measurements have been made in the stratum corneum. See, e.g., Hanson, K. M., et al., Two-photon fluorescence lifetime imaging of the skin stratum corneum pH gradient. Biophysical Journal, Vol. 83, pp. 1682-1690. An alternative molecule is benzenedicarboxylic acid, 2(or 4)-[10-(dimethylamino)-3-oxo-3H-benzo[c]xanthene-7-yl]-(SNARF-1) using 514 nm excitation and fluorescence detection at 640 nm and 587 nm, respectively. The fluorescence ratio at these emission wavelengths allows the determination of the ratio of the protonated and unprotonated forms of the dye. This ratio allows the determination of the pH of the dye environment using the Henderson-Hasselbalch equation (9).

$$pH = pKa - log \frac{HA}{{A}^{-}} \qquad ( 9 )$$

As an illustrative example, for BCECF this relationship becomes equation (10):

$$pH = pKa + log ( \frac{( \frac{{F}_{490}}{{F}_{439}} ) - ( \frac{{F}_{490}^{a}}{{F}_{439}^{a}} )}{( \frac{{F}_{490}^{b}}{{F}_{439}^{b}} ) - ( \frac{{F}_{490}}{{F}_{439}} )} ) \qquad ( 10 )$$

In this expression the fluorescence is monitored at a wavelength of 535 nm, the terms F₄₉₀ and F₄₃₉ refer to the fluorescence intensity monitored at excitation wavelengths of 490 nm and 439 nm respectively and the terms with superscripts a and b represent the limiting values of the fluorescence ratio in acid (a) and base (b) respectively.

Use of the dye to measure absolute values of pH requires a small correction of the fluorescence ratio since the two fluorescence emission bands are not completely separated.

A difference comparison of both intracellular and extracellular pH measurements allows measurements to be made of lactate synthesis, transport and diffusion out of the interstitial fluid. The difference between intracellular and extracellular pH is indicative of the hydrogen ion produced within the cell (due to glycolysis) and those hydrogen ions that are produced systemically.

Estimation of the Effect of Glucose Metabolism on Changes in Intracellular pH

Numerous prior studies measured intracellular pH in a variety of organisms and cell types. See, e.g., Roos, A. and Boron, W. F. (1981) Intracellular pH. Physiological Reviews, vol. 61, pp. 297-434. Of interest are experiments that examine the effect of weak acids and bases on the pH of cell extracts and homogenates. Using a simple equation (11) from Michaelis to describe the buffering capacity of a solution, the physicochemical buffering of these samples can be expressed as “intracellular buffering power” as follows:

$$β = \frac{ⅆ ( A or B )}{ⅆ ph} \qquad ( 11 )$$

β: total buffering power of intracellular fluid

A: amount of added acid

B: amount of added base

See, e.g., Roos and Boron, 1981, pp. 389-400.

The intracellular buffering power of different tissues and cell types are summarized in Roos and Boron (1981) supra Table 13, at p. 399. Table 2 (below) uses equation (11) and the intracellular buffering power of rat tissues to calculate the potential effect of 5 mM glucose (undergoing glycolysis) on intracellular pH. Information on skin (for any organism) was not available in the art. These calculations also are based on studies reporting that a net of two protons (two lactate) are produced for every molecule of glucose that is metabolized. See e.g., Busa, W. B. and Nuccitelli, R. (1984) Am. J. Physiol., vol. 246, R409-R438; and Robergs, R. (2001), Professionalization of Exercise Physiology-Online. vol. 4, no. 11. Thus, by deriving this information for a specific cell type and for the conditions of an individual subject, the glucose available to a cell for glycolysis can be calculated from the measured pH.

TABLE 2

| | | pH change (5 mM glucose |
|---|---|---|
| Tissue | Buffering Power | or 10 mM H⁺) |
| Rat Brain (whole) | 18.5 | −0.54 |
| Rat Diaphragm Muscle | 67 | −0.15 |
| Rat Skeletal Muscle | 66-68 | −0.15 to −0.16 |
| Rat Cardiac Muscle | 51 | −0.19 |
| Rat Ventricular Muscle | 77 | −0.13 |

Measurement Protocol

The rationale for making measurements of D-glucose and other simple sugars using pH (ie., lactate/H⁺) sensitive intracellular dyes is described. The specific rationale is based upon the concept that glycolytic mechanisms may be monitored via metabolite concentration to give an estimation of the total D-glucose available to the cell (Scheme 1, FIG. 17A). In this invention, the fluorescence of a pH-sensitive dye is used to determine blood glucose concentrations. This measurement is possible because for every glucose molecule undergoing glycolysis, two lactate/H⁺ molecules result. Thus, depending upon the buffering capacity for any specialized cell types, the pH is indicative of the quantity of glucose available. During glycolysis, the glucose is immediately converted to lactate.

In most embodiments, two steps are required for the glucose measurement. The measurements to be made are the intensity of fluorescence at about 580 nm and 640 nm with 532 nm excitation. The bandwidth of these measurements is typically 10 nm wide. Intracellular pH is monitored using an intracellular dye that is equivalent or superior in efficacy to SNARF 5 AM; i.e., extracellular pH is monitored using an extracellular dye equivalent or superior in efficacy to SNARF 5 (SNARF®-5F 5-(and-6)-carboxylic acid). The dyes are typically applied in two different places. A third spot is applied using an intracellular dye equivalent or superior in efficacy to SNARF 5 AM to be used to determine the spectra of the acidic and basic forms of the dye. All dyes are applied in a 10 μL volume having a final concentration of 200 μM.

The protocol used for application of the dye requires that a skin temperature between 30° C. and 37° C. be maintained. The area of skin to be measured is rinsed with approximately 1 mL of distilled water and wiped dry with an uncoated tissue or Kimwipe. It is preferable to wipe the area clean with a clinical alcohol wipe. Once a clean area of skin has been prepared, 10 μL of dye is applied to the skin with an automatic dispenser or pipette. The dye spots are protected from room light and all manipulations are carried out under dimmed room light. After one hour, any dye that remains on the surface of the skin is blotted off with a Kimwipe. The uptake of dye was monitored using a two-photon microscope and by measuring spectra after the dye is applied. It was determined by observation that measurement should begin three hours following application of the SMMR.

To test the efficacy of the sensor measurement during the measurement period, sensor readings were recorded every time a blood sample was withdrawn for reference measurements. The test measurement was designed so that the autofluorescence, and the fluorescence from SMMRs located in the intra and extracellular spaces can be acquired ideally at the same time. The only way to do this at present is to move the sensor to different sites between measurements. However, other methods may be used as they become available to provide equivalent information.

Reference blood samples drawn were analyzed for blood glucose, lactate and hematocrit. Spectra were acquired from the skin, to obtain autofluorescence, from the spot where an extracellular dye equivalent or superior in efficacy to SNARF 5 was applied to obtain the extracellular pH, and from the spot where an intracellular dye equivalent or superior in efficacy to SNARF 5 AM form (available from Molecular Probes, Inc. Eugene, Oreg.) was applied to obtain the intracellular pH. Finally, acid and base were applied to the control spot to obtain the spectra from the fully protonated and fully deprotonated dyes.

For normal prandial studies, this measurement protocol lead to a smaller range of glucose values than those obtained using clamp studies. Typically, fasted mammals have a blood glucose concentration of anywhere between 50 mg/dL to 100 mg/dL, whereas fed mammals have a glucose concentration range of 100 mg/dL to 150 mg/dL.

The data were analyzed according to equation (12):

$$pH = {pK}_{A} + log [ \frac{R - {R}_{B}}{{R}_{A} - R} \times \frac{{F}_{B ( λ 2 )}}{{F}_{A ( λ2 )}} ] \qquad ( 12 )$$

where R is the ratio of the fluorescence intensity at 580 nm and 640 nm, R_{B} is the same ratio when the dye has been made alkaline and R_{A} is when the dye has been acidified. The terms F_{A} and F_{B} are the intensity measurements at 640 nm in acid and base respectively.

Equation (12) is a modified version of the Henderson-Hasselback equation that describes the fraction of molecules that are protonated in an acid-base system at a certain pH. The term in parentheses is inversely proportional to the hydrogen ion concentration. The ability to relate glucose concentration to pH is based on the stoichiometry of glycolysis. For every equivalent of glucose that is metabolized, two equivalents of hydrogen ions are generated. pH is simply the negative log of the hydrogen ion concentration.

The fluorescence ratio values were obtained after the intensity of the autofluorescence has been subtracted. Although this expression actually gives the pH value in these measurements, it should be realized that the glucose concentration is only a function of the pH. If the oxidation of glucose results in the formation of hydrogen ions then it is the corrected fluorescence ratio that is important in the determination of glucose concentration. As far as the influence of external pH, it is surmised that the changes in intracellular pH are dependent on the difference between intracellular and extracellular pH. The basis for this assumption is that the monocarboxylate transporter is a facilitated diffusion pump. As a result, hydrogen ions can be pumped out if the external pH is high compared to the intracellular pH. It is more difficult to pump hydrogen ions out if the pH is low.

### EXAMPLE 5

Empirical Calibration Scheme—General Case

An empirical correction scheme for obtaining quantitative fluorescence spectra from molecules embedded within the skin of individual human subjects is required due to the unique scattering and absorptive properties of individuals. The effects on fluorescent spectra brought about by these individualistic optical properties include changes in bandshape and relative fluorescence intensity. A general equation (13) for obtaining quantitative fluorescence calibration spectra, which will accommodate for unique tissue matrix effects, is written as:

$${\hat{C}}_{i} = ( 𝔍 - {𝔍}_{B} ) \frac{{c}_{1} - {c}_{2}}{( {f}_{1} - {𝔍}_{B} ) - ( {f}_{2} - {𝔍}_{B} )} \qquad ( 13 )$$

where Ĉᵢ is the estimated concentration for a test sample I; ℑ is the fluorescence response of the test sample I; ℑ_{B} is the fluorescence response of the test sample site with solvent treatment only; f₁ is the fluorescence response of the sample site at concentration c₁ (a concentration higher than the expected concentration of the test sample I); f₂ is the fluorescence response of the sample site at concentration c₂ (a concentration lower than the expected concentration of the test sample I). See, e.g., Harrison, G. R., Lord, R. C., and Loofbourow, J. R. Practical Spectroscopy, Prentice-Hall, Inc., New York, N.Y., 1948, pp. 412-414.

### EXAMPLE 6

Empirical Calibration Scheme—Special Case of Lactate/H+: pH Measurements

Specifically for the case involving quantitative determination of lactate/H⁺ using intracellular or extracellular pH measurements, one would work with hydrogen ion concentration directly as [H⁺]. In the case where an indicator dye exhibits a fluorescence response due to a change in [H⁺] following the relationship as shown in equation (14):

$$[ {H}^{+} ] = {k}_{a} ( \frac{\frac{f ( {λ}_{1} )}{f ( {λ}_{2} )} - \frac{{f}_{B} ( {λ}_{1} )}{{f}_{B} ( {λ}_{2} )}}{\frac{{f}_{A} ( {λ}_{1} )}{{f}_{A} ( {λ}_{2} )} - \frac{f ( {λ}_{1} )}{f ( {λ}_{2} )}} ) \cdot ( \frac{{f}_{B} ( {λ}_{1} )}{{f}_{A} ( {λ}_{2} )} ) \qquad ( 14 )$$

where f(λᵢ) is the fluorescence measurement at wavelength i and the subscripts A and B represent the respective acidic and basic endpoints using a titrimetric approach. See, e.g., Molecular Probes Product Information Sheet #MP 01270, SNARF pH Indicators, Molecular Probes, Eugene, Oreg., Oct. 22, 2002). This relationship, shown in equation (15), holds noting that background correction is applied to each fluorescent signal prior to ratio calculation. If λ₂ is selected as the isosbestic point, then the relationship below holds. For a dye such as SNARF-1: λ₁=580 nm, λ₂=640 nm, and λ_{EX}=514 nm, λ_{Isosbestic}=608 nm.

$$[ {H}^{+} ] = {k}_{a} ( \frac{\frac{f ( {λ}_{1} )}{f ( {λ}_{2} )} - \frac{{f}_{B} ( {λ}_{1} )}{{f}_{B} ( {λ}_{2} )}}{\frac{{f}_{A} ( {λ}_{1} )}{{f}_{A} ( {λ}_{2} )} - \frac{f ( {λ}_{1} )}{f ( {λ}_{2} )}} ) \qquad ( 15 )$$

Then the corrected equation 13 for measurement of hydrogen ion concentration accounting for matrix effects should be as equation (16):

$${[ {\hat{H}}^{+} ]}_{i} = ( 𝔍 - {𝔍}_{B} ) \frac{{[ {H}^{+} ]}_{1} - {[ {H}^{+} ]}_{2}}{( {f}_{1} - {𝔍}_{B} ) - ( {f}_{2} - {𝔍}_{B} )} \qquad ( 16 )$$

where [Ĥ⁺]ᵢ is the estimated concentration for a test sample i; ℑ is the fluorescence response of the test sample i; ℑ_{B} is the fluorescence response of the test sample site with solvent treatment only; f₁ is the fluorescence response of the sample site at concentration [Ĥ⁺]₂ (a concentration higher than the expected concentration of the test sample i); f₂ is the fluorescence response of the sample site at concentration [Ĥ⁺]₂ (a concentration lower than the expected concentration of the test sample i).

### EXAMPLE 7

Use of External Calibration Standards for General Case

The use of external calibration standards (i.e., standard addition) is essential in providing a bloodless method for calibrating in vivo measurements. In theory, a set of two or more calibration standards comprised of known concentrations of analytes (e.g., glucose) can be externally added to tissue and delivered to the specific analysis target site(s). Such a practice does not rely on a purely theoretical approach dependent on some fixed assumption set. Thus a more broadly applicable method would involve reliance on an empirical measurement approach. Such an approach must be applied across individualistic physiological properties of specific tissue sites including: perfusion rate, interstitial fluid volume, rates of diffusion into and out of the tissue, glucose transport, and the like. Such phenomena can be illustrated generally as in Scheme 4 (FIG. 17D) illustrating fluid issues related to in vivo skin calibration.

As an example, an in vitro experiment using such standard addition can be reviewed. The experiment is to determine the final concentration of a cuvet initially containing a liquid of 100 volume units (Vi) and a concentration of 100 w/v (Ci). In this case, neither the initial volume nor the initial concentration is known. To begin, a known Standard liquid (A1) is added to the cuvet having a volume of 100 volume units (Va1) and a concentration of 0.0 w/v (Ca1). A fluorescence measurement is made of the solution plus Standard A1 and the result recorded as ℑa1. The final concentration of the cuvet at this point may be determined using the general equation (17):

$${C}_{{f}_{a1}} = \frac{( {C}_{i} \cdot {V}_{i} ) + ( {C}_{a1} \cdot {V}_{a1} )}{{V}_{i} + {V}_{a1}} = \frac{( 100 \cdot 100 ) + ( 0.0 \cdot 100 )}{100 + 100} = 50 \frac{w}{v} \qquad ( 17 )$$

A second Standard liquid (A2) is then added to the cuvet having a volume of 100 volume units (Va2) and a concentration of 500 w/v (Ca2). A fluorescence measurement is made of the solution plus Standard A2 and the result recorded as ℑa2. Following the addition of Standard A2 the cuvet now contains a concentration calculated using equation (18):

$${C}_{{f}_{a2}} = \frac{( {C}_{i} \cdot {V}_{i} ) + ( {C}_{a2} \cdot {V}_{a2} )}{{V}_{i} + {V}_{a2}} = \frac{( 50 \cdot 200 ) + ( 500 \cdot 100 )}{200 + 100} = 200 \frac{w}{v} \qquad ( 18 )$$

If a fluorescence method has been developed capable of measuring the concentration of analyte defined as a linear relationship over a concentration range of between 50 and 400 w/v (see Table 3), then equation (19) holds as:

$$\frac{{𝔍}_{a2}}{{𝔍}_{a1}} = \frac{{C}_{{f}_{a2}}}{{C}_{{f}_{a1}}} \qquad ( 19 )$$

Thus a ratio measurement of ℑa2 and ℑa1 yields a value of 200/50=4.0 and provides sufficient information to compute absolute concentration of the initial fluid as well as the final fluid levels. The examples below consider two examples of the in vivo case.

### EXAMPLE 8

Equivalent volumes of Standards A1 and A2 are added to the tissue as volumes Va1 and Va2; these volumes approximate the current interstitial volume. The concentration of the added Standards A1 and A2 are 0.0 w/v (Ca1) and 300 w/v (Ca2). The interstitial volumes are assumed to remain approximately the same as the liquid from the Standards mix with the interstitial fluid, i.e., after a period, there is a mixing of liquids causing an equilibrium of the analyte levels, but no overall interstitial fluid volume change. The equivalent relationships can be calculated for any set of assumptions. Since in this case the equivalent volume assumption is made then equation (20) holds:

$${C}_{{f}_{ai}} = \frac{( {C}_{i} \cdot {V}_{i} ) + ( {C}_{ai} \cdot {V}_{ai} )}{{V}_{i} + {V}_{ai}} \qquad ( 20 )$$

Equation (20) reduces to a simple relationship where the volumes Vi, Va1 and Va2 are equivalent and there is assumed diffusion of the analyte from the Standards to the interstitial fluid equilibrating the concentration given sufficient time. Thus, equation (21) is used:

$${C}_{{f}_{ai}} = \frac{{C}_{i} + {C}_{ai}}{2} \qquad ( 21 )$$

This scenario takes into consideration that the addition of the first Standard A1 at 0.0 w/v concentration reduces the concentration; and then the second Standard A2 at 300 w/v concentration is added. As such the following Table 3 holds.

TABLE 3
Application of Standards A1 and A2 as 100 unit volume and
0.0 w/v and 300 w/v concentration.

| Initial Volume (Vi) | Initial Conc. (Ci) | C_{f}_{ₐ₁} | C_{f}_{ₐ₂} | $\frac{{C}_{{f}_{a2}}}{{C}_{{f}_{a1}}} = \frac{{𝒯}_{a2}}{{𝒯}_{a1}}$ |
|---|---|---|---|---|
| 100 units | 50 | 25 | 162.5 | 6.5 |
| 100 units | 100 | 50 | 175 | 3.5 |
| 100 units | 150 | 75 | 187.5 | 2.5 |
| 100 units | 200 | 100 | 200 | 2.0 |
| 100 units | 250 | 125 | 212.5 | 1.7 |
| 100 units | 300 | 150 | 225 | 1.5 |
| 100 units | 350 | 175 | 237.5 | 1.36 |
| 100 units | 400 | 200 | 250 | 1.25 |

### EXAMPLE 9

Equivalent volumes of Standards A1 and A2 are added to the tissue as volumes Va1 and Va2; these volumes approximate the current interstitial volume. The concentration of the added Standards A1 and A2 are 0.0 w/v (Ca1) and 400 w/v (Ca2). As in Example 8, the interstitial volumes are assumed to remain approximately the same as the liquid from the Standards mix with the interstitial fluid, i.e., there is a mixing of liquids causing an equilibrium of the analyte levels, but no overall interstitial fluid volume change. Since this assumption is made, then equations 20 and 21 are used.

This scenario takes into consideration that the addition of the first Standard A1 at 0.0 w/v concentration reduces the concentration; and then the second Standard A2 at 400 w/v concentration is added. As such the following Table 4 holds as.

TABLE 4
For the application of Standards A1 and A2 as 100 unit volume and
0.0 w/v and 400 w/v concentration.

| Initial Volume (Vi) | Initial Conc. (Ci) | C_{f}_{ₐ₁} | C_{f}_{ₐ₂} | $\frac{{C}_{{f}_{a2}}}{{C}_{{f}_{a1}}} = \frac{{𝒯}_{a2}}{{𝒯}_{a1}}$ |
|---|---|---|---|---|
| 100 units | 50 | 25 | 212.5 | 8.5 |
| 100 units | 100 | 50 | 225 | 4.5 |
| 100 units | 150 | 75 | 237.5 | 3.17 |
| 100 units | 200 | 100 | 250 | 2.5 |
| 100 units | 250 | 125 | 262.5 | 2.1 |
| 100 units | 300 | 150 | 275 | 1.83 |
| 100 units | 350 | 175 | 287.5 | 1.64 |
| 100 units | 400 | 200 | 300 | 1.5 |

### EXAMPLE 10

Screening and Optimizing Organic Dyes for SMMR Activity

In some embodiments of the invention, a dye is added into a tissue with an anticipated SMMR-response activity, and spectra are collected for a set of predetermined excitation and emission wavelengths. The excitation wavelength set selected corresponds to the maximum absorption spectrum of the dye being used. The optimal measurement wavelength for excitation and emission is then determined empirically for each SMMR application such that the selected excitation wavelength results in a combined effect where maximum emission intensity and response is achieved for each metabolite of interest. Metabolites useful for tracking glucose were derived from an understanding of the glycolytic pathway for the cells of interest and an understanding of which dyes may actually behave as SMMRs for quantitative reporting of these metabolites. By selecting the optimum wavelengths for SMMR measurement in an empirical fashion, the precise method for quantitative detection of each metabolite was achieved, thereby yielding maximum analytical selectivity, repeatability, and reproducibility.

Empirical Procedure for the Development of Calibration Protocols

In an exemplary embodiment, the following procedure is used to develop the calibration protocol for a blood glucose analysis method combining SMMRs with a low-cost, handheld sensor. The procedure follows the steps of: (1) the glucose (or another blood or tissue analyte) is measured for the test subject (or series of test subjects) by withdrawing blood from a subject and by analysis via a reference blood glucose measurement (the glucose may be intentionally varied within the test subject for the test evaluation period); (2) the metabolic reporter signal and a marker (or reference) wavelength signal are measured at a time-stamped interval corresponding to the blood glucose reference measurement (this is completed for a series of excitation and emission wavelengths); (3) the ratio of the metabolic reporter and reference or marker wavelengths is calculated for each set of excitation and emission wavelengths; (4) the series of ratio measurements of the reporter/reference is compared to the reference blood glucose measurements; (5) the optimum wavelength sets are derived and the absolute ratios determined that best correspond to specific blood glucose levels, taking into account the lag times and best mathematical model; (6) a small handheld device is provided, preferably where the device has the capability to measure the signal at the optimized specific wavelengths using exact excitation sources and emission detection schemes (with defined intensity and bandshape characteristics); (7) the ratio measurements of the device when coupled with specific SMMRs produces a metabolite profile that is used to directly predict blood glucose concentration using algorithms described herein. Those skilled in the art will recognize that when the metabolic pathways for multiple biosyntheses are defined, that this empirical testing method can be used to screen multiple dyes for their efficacy as SMMRs for a variety of metabolic measurements. Thus, without a great deal of knowledge about specific pharma-kinetic activity or dyes, a series of compounds can be screened and optimized for SMMR activity. All dye candidates to be tested for SMMR activity in humans are first screened properly to ensure safety.

### EXAMPLE 11

Factors Affecting the Molecular Structure and Action of Organic Dyes Suitable for use as SMMRs

Molecular Design

There are six main characteristics of a dye molecule that determine its efficacy as an SMMR in this application. These include: (1) its affinity and specificity for target cells and cell structures; (2) its binding properties and residence time in skin; (3) its safety to cells and organisms; (4) its speed of delivery; (5) its specificity for the metabolite of interest; and (6) its spectral properties. Properties that control the affinity and specificity for target cells and cell structures for SMMR molecules into skin cells include:

1. The partition coefficient in octanol/water together with the solubility in aqueous solution, which determines how the molecule is distributed between the aqueous and lipid phases in the tissue;

2. The charge, which affects electrostatic interactions of the compound;

3. The vapor pressure at 25° C., which determines the evaporation rate at the skin surface;

4. The molecular size, which controls the diffusion of the material through a porous interface or a viscous liquid.

Factors affecting the functionality of the molecule include the reactivity and reduction potential of the molecule, the pKa and the energy level of the first excited state.

Spectral properties that are important for SMMRs include the absorption spectrum of the chromophore, the fluorescence spectrum and the emission quantum yield. Properties that moderate the absorption characteristics include the degree of conjugation in the molecule, the number of electrons in the conjugated system and the electro-negativities of substituents attached to the molecule. Factors that affect the fluorescence emission spectrum are similar to those that affect the absorption spectrum. The fluorescence quantum yield, which determines the intensity of the fluorescence, is influenced by the flexibility of the molecule and the intramolecular reactivity.

An example of how a xanthene dye may be modified to act as a long wavelength pH sensitive dye for specific action as a lactate/H⁺ SMMR is described herein. One possible structure is shown in Scheme 3 (FIG. 17C). As shown in FIG. 17C, the xanthene ring is substituted with hetero atoms (A) that extend the conjugation of the molecule across the fused ring system. Electron density in the ring system is increased by the lone pair of electrons on the heteroatom that are partly delocalized into the ring system. Typical groups at these positions would include an amine and an amide, such as rhodamine.

The pKa of the molecule is controlled by the substitution of acidic and basic groups (B) and the nature of the heteroatoms (A). Small changes to the pKa may be made by substitution of electron donating or withdrawing groups to the ring (D). The quantum yield of fluorescence (φ_{F}) and hence the intensity of the fluorescence is determined by the balance between the rate constants for radiative (kᵣ) and non-radiative (kₙᵣ) decay as shown in equation (22):

$${φ}_{F} = \frac{{k}_{r}}{( {k}_{r} + {k}_{nr} )} \qquad ( 22 )$$

The radiative rate constant is determined by the probability of a transition whereas the non-radiative rate constant is affected by the number of modes of vibration that a molecule has and any intramolecular reactivity that can quench the excited state.

The absorption spectrum of the molecule is determined by the extent of the conjugation as well as substitution on the ring (C). Substitution of both electron withdrawing and electron donating groups in a push-pull type of system extends the overall conjugation of the system and causes a bathochromic shift (to longer wavelengths) of the spectrum. A number of empirical rules have been put forward to predict spectra. The well-known Woodward rules, for example, predict that for a simple conjugated system the addition of a double bond adds about 30 nm to the wavelength maximum.

The polarity of the molecule can be altered, without grossly affecting other properties of the molecule, by substitution of non-conjugated groups to the ring system (E). Many xanthene dyes are synthesized with a substituted phenyl ring at R₂. It is by specific modification of this dye and the measurement of its fluorescence signature that allows the dye to function as an SMMR to relate lactate/H⁺ to D-glucose concentration (as noted in FIGS. 1-18 and Table 1).

### EXAMPLE 12

Use of Glycogen Particle Density

The measurement of glycogen particle numbers indicates a direct proportionality to the amount of glucose in the metabolic pathway of the cell (for an individual metabolic rate). As Scheme 2 (FIG. 17B) illustrates, a measurement of glycogen synthesis provides an indicator of glucose concentration because the only biochemical route to glycogen is directly from glucose. The use of iodine-based SMMRs within the skin can be measured using an optical reader as a direct indicator of glycogen particle concentration in the skin. Skin glycogen concentration can be related to skin glucose levels, which in turn are mathematically related to blood glucose levels. Thus, skin glycogen concentration yields direct information on skin and blood glucose concentrations. The parameter of glycogen particles can be measured using a variety of known techniques. Glycogen particles are known to have a mean particle size diameter of approximately 30 nanometers (nm). Thus, an ideal wavelength for characterizing the presence of these particles in an absorptive-scattering media such as skin would be at 2.5 to 3.5 times the diameter or approximately 75 to 105 nm ultraviolet light. This invention contemplates utilizing such a wavelength to characterize the number of glycogen particles within the skin, as well as utilizing other potential methodologies for measuring the particle density for glycogen include scattering measurements in the 290 nm to 750 nm spectral regions, and includes optical coherent tomography. Mathematical manipulations of the data derived from these techniques can provide correlative information allowing prediction of glycogen particle numbers.

In a specific embodiment, a series of techniques are described in the invention which allow the placement of a specialized tattoo, comprised of at least one of a choice of specific glycogen indicating SMMRs, into the epidermis for analysis of mitochondrial membrane potential and pH indicating signals. Measurement of glycogen particles, which preferentially absorb SMMRs, is monitored directly using an optical scattering reader. The optical reader calculates the total absorption of the SMMR into the glycogen particles. Once determined, the glycogen content of the skin is empirically related (by first principles mathematical models) to reference skin and blood glucose levels. Simultaneously, a quality value is calculated, which tells the user the quality of the glucose value reported. Based on this quality value, the user may be instructed to make one or more additional measurements until the quality value is indicative of an accurate result.

Once the tattoo or mark is produced, this invention may be fully sufficient for in vivo noninvasive determination of the rate of glucose utilization within living human epidermal cells as long as the SMMR remains within the stratum spinosum. SMMRs meeting the requirements for this embodiment are described above, and include, e.g., iodine dissolved in potassium iodide. Iodine forms a blue-black complex with glycogen, the intensity of which is directly related to the number of particles of glycogen present in the tissue. The visible response of these SMMRs is then related to blood glucose level by the relationship given in equation (23):

$$[ G ] ∝ \frac{\# glycogen particles . \times NAD ( P ) H}{FAD \times NO \times pH \times {O}_{2}} \qquad ( 23 )$$

Equation (23) is based on measuring a cell function and normalizing this function for the relative metabolic rate of the tissue. The number of glycogen particles is directly related to the glucose concentration. This relationship will break down when metabolism is high and all the glycogen reserves have been utilized. The concentration of glycogen particles can be obtained from measurements using optical coherent tomography, light scattering, or differential staining of glycogen particles using iodine stains.

### EXAMPLE 13

An Example of a Targeted Pathway

Mathematical Modeling Applications to Glucose Concentration

FIG. 18 is a proportionality-qualitative description of how the glycolytic pathway (e.g., glycolysis) relates to glucose concentration in cellular metabolism. The quantitative description of these pathways is developed dependent upon accurate, selective, and responsive measurement parameters yielding indirect or direct information for glucose concentration. An example of the quantitative treatment for fluorescence changes associated with the activity of glucose oxidase is given in Equation (24).

Several examples of the mathematical models required for fitting the reported glucose to the measured blood glucose for this invention are given in Equations 1-5. The addition of glucose to a solution of glucose oxidase causes an increase in fluorescence after a lag time. The lag period can be related to the concentration of the glucose oxidase, the oxygen concentration and the glucose concentration. Assuming that the rate constant for the reoxidation of the reduced enzyme is significantly greater than the binding and oxidation of glucose, and that the concentration of the free oxidized enzyme is higher than that of other forms before the time at which the fluorescence changes, then the following expression in equation (24) has been derived.

$${t}_{m} - {t}_{0} = \frac{1}{{{k}_{1} [ {GO}_{x} ]}_{0}} ln ( \frac{{[ G ]}_{0}}{{[ G ]}_{0} - {2 [ {O}_{2} ]}_{0}} ) \qquad ( 24 )$$

where

tₘ Time at which the fluorescence changes

t₀ Time at which glucose is introduced

k₁ Rate constant for the reduction of GOₓ by glucose

[GOₓ]₀ Initial concentration of glucose oxidase

[G]₀ Initial concentration of glucose

2[O₂]₀ Initial concentration of oxygen

See, e.g., Sierra J. F., Galban J., Castillo, J. R. “Determination of Glucose in Blood Based on the Intrinsic Fluorescence of Glucose Oxidase.” Anal. Chem. 1997 69(8), 1471-1476).

### EXAMPLE 14

Other Monitoring Techniques and Metabolites

Lactate Transport

Lactate transport is monitored by measuring intracellular and extracellular pH using fluorescent SMMRs, as previously described. The xanthene dye BCECF has been used to monitor lactate transport in a number of tissues (see e.g., Carpenter, L. and Halestrap, A. P. 1994 Biochem. J. 304, 751-760). In the present invention this dye is used to monitor both intracellular and extracellular pH. The extracellular pH is monitored to measure variations in physiology within the body that are unrelated to glucose metabolism in the epidermis, but are related to metabolic pH changes in the body. The intracellular pH, as measured, is then corrected using the value of the measured extracellular pH.

Oxidative Phosphorylation

Oxidative phosphorylation can be monitored by NADH fluorescence. This fluorescence is measured in the presence and absence of oxygen. These two measurements yield the rate of oxidative phosphorylation and a measure of the overall metabolism of the cell. The rate of oxidative phosphorylation is dependent on the overall substrate availability to the cell, which requires oxygen. In the absence of oxygen, the overall metabolism is dependent on glycolysis alone.

The oxidative phosphorylation pathway for glucose is determined by measuring oxygen consumption along with the NADH/FAD fluorescence ratio. This ratio has been used in the past to determine the overall reduction potential of the cell. The measurement of the oxygen consumption rate determines the rate of oxidative metabolism in the tissue. The sensitivity of the NADH/FAD fluorescence ratio can be increased by the use of an energy transfer or redox potential measuring dye to amplify overall signal intensity. An example of such a dye suitable for use as an SMMR is rhodamine 123, although other compounds containing conjugated aromatic systems can also be used.

In a preferred embodiment, the amplifying SMMR molecule is positively charged at pH 7 and has a high quantum yield of fluorescence. In a further embodiment, the SMMR molecule has little absorption in the region where NADH absorbs. Excitation of NADH results in energy transfer to the SMMR dye that then fluoresces with efficiency at least ten times greater than that of NADH alone.

Photobleaching

Photobleaching is a process that occurs with virtually all fluorescent dyes. The term is something of a misnomer since it literally means the loss of color as a result of irradiation by light. The loss of color is the result of a photochemical reaction that results in a new chemically distinct compound being formed that does not exhibit the same fluorescence properties as the parent SMMR compound. This new photo-degraded compound will have altered photophysics compared to the parent molecule but its properties are not necessarily loss of color. Photobleaching is a hindrance to continuous fluorescence-based monitoring and is exacerbated by the presence of oxygen, high concentrations of reactive species and high light levels. For this present invention SMMRs are used with high quantum yields of fluorescence (which implies that the main process for deactivation of the excited state is fluorescence), and they are excited with the minimum amount of excitation light. Photoreactivity is also reduced by the low oxygen tension in the skin.

Differential Monitoring

The mechanism presented in Scheme 1 (FIG. 17A) for the measurement of glucose requires that the majority of glucose be metabolized by glycolysis because oxidative phosphorylation may also utilize fatty acid metabolites as substrates instead of glucose. Oxidative phosphorylation in skin comprises only ˜2% of metabolism and this fraction may be controlled by reducing the oxygen available to the cells, although experimental data suggests that there is little or no effect of oxygen concentration on glycolysis. By performing a differential measurement with and without oxygen, the fraction of glycolytic and oxidative metabolism is determined.

Glycolysis

In tissues that undergoes primarily anaerobic metabolism (i.e., glycolysis) the products of the glycolysis reaction pathway are lactate and adenosine triphosphate (ATP). ATP is synthesized from ADP, the diphosphate analog, and a phosphate. Lactate is generated as a waste product of the pathway. The lactate concentration within the cell is dependent on lactate transport out of the cell and on the rate of glycolysis. The extracellular lactate concentration is dependent on lactate transport and diffusion of lactate into the blood stream. The production of lactate correlates with the intracellular pH. The pH of epidermal tissue, using intra- and extracellular pH sensitive SMMRs, can be used to specifically relate intracellular pH changes to glucose utilization via glycolysis. The use of NMR techniques using phosphorous (³¹P) and proton (¹H) probes allows the measurement of ATP, phosphate, pH and lactate simultaneously. This technique alone can be used to determine the relationship between glucose concentration and glycolysis. The use of ³¹P NMR is described specifically for measuring the effect of exercise on the levels of ATP, phosphocreatine, and orthophosphate in human forearm muscle. See e.g., G. K. Radda. Science 233: 641 (1986). pH can also be measured in vivo and directly using ³¹P NMR. See e.g., citations in D. G. Gadian et al. I_{N}: Biological Applications of Magnetic Resonance, R. G. Shulman, ed., (Academic Press, 1979), p. 475. The ³¹P magnetic resonance technique also provides information on the orthophosphate concentration for glucose metabolism in the Kreb's cycle and/or oxidative phosphorylation pathway. The lactate/pyruvate ratio and the β-hydroxybutyrate/acetoacetate ratios have been used to estimate cytosolic and mitochondrial NADH/NAD(P)H ratios respectively. See e.g., Tischler, M. E., et al., Arch Biochem Biophys, 1977. 184(1): p. 222-36; Poole, R. C. and A. P. Halestrap, Am J Physiol, 1993. 264(4 Pt 1): p. C761-82; Groen, A. K., et al., J Biol Chem, 1983. 258(23): p.14346-53.

Nitric Oxide (NO):

NO has been shown to correlate inversely with glucose concentration. This reactive molecule acts as a vasodilator and interacts with thiol groups. The reaction of NO with hemoglobin has also been monitored in the past using absorption spectroscopy. NO may also be measured using an NO meter using a probe head that is as small as 30 μm.

Scheme 1 (see FIG. 17A) points to the measurement sites required to define the glucose metabolism in epidermis thereby providing complete information for the fate of glucose metabolized in the skin. NO causes physiological effects such as vasodilatation and is a reactive material that interacts with thiols and the basement membrane of the dermal/epidermal junction. Direct measurement of NO is possible using commercially available technology. The measurement of NO will be used, if necessary, for final correction of the glucose concentration. The determination of the NO correction follows initial comparisons of blood glucose estimated from fluorescence measurements when compared to blood glucose measured using a reference technique (e.g., YSI Incorporated, PO Box 279, Yellow Springs, Ohio 45387 USA). The change in glucose concentration as affected by NO concentration is described in the equation (23). The use of NO concentration information for final blood glucose correction is also described herein. When required, equations (1-5) of the invention are modified by the addition of an NO term as shown in equation (25). This adjustment accounts for the cases where NO alters the perfusion rate significantly.

$$[ {Glucose}_{blood} ] = f ( \frac{{𝔍}_{Reporter}}{{𝔍}_{Reference}} ) \cdot {k}_{i} ( \frac{1}{[ NO ]} ) \qquad ( 25 )$$

Where,

$$f ( \frac{{𝔍}_{Reporter}}{{𝔍}_{Reference}} )$$

is the in vivo fluorescence signal ratio of reporter fluorescence to reference (or marker) fluorescence varying with respect to changes in glucose concentration within the measured target tissue; and kᵢ is the computed weighting factor attributing the effect of NO concentration on the perfusion rate. The factor kᵢ is computed empirically following comparisons of blood glucose optically determined versus reference values using standard regression methods (see for example H. Mark and J. Workman, Statistics in Spectroscopy, 1^{st} Ed., Academic Press, 1991; and 2^{nd} Ed., Elsevier Publishers, 2003)

### EXAMPLE 15

Consideration of Blood Glucose Concentration and Fluorescence

Previous work has demonstrated that the lag time between blood glucose levels and non-perturbed epidermis is 2.9 to 4 percent per minute for the differential concentrations (vis-à-vis blood and epidermal glucose concentrations). See, e.g., J. M. Ellison et al. Diabetes Care, June 2002, 25(6), 961-964; B. M. Jensen et al. Scandinavian Journal of Clinical Laboratory Investigation, 1995, 55, 427-432; P. J. Stout, Diabetes Technology & Therapeutics 2001, 3(1), 81-90; C. P. Quinn, Publication 0193-1849/95 The American Physiological Society, E155-E161). In practice, a 5 to 15 minute lag is most often experienced between real-time measured blood glucose levels and glucose levels determined at the keratinocyte/epidermal layers. The fingertip area keratinocyte/epidermal layers are considered ideal due to their high vascularization. The time required for the epidermis to reach an equilibrium with blood glucose at steady-state, dependent on the measurement site, has been reported to be from 25 to 35 minutes. See, e.g. K. Jungheim and T. Koschinsky Diabetes Care, 25(6), 956, 2002; and J. Ellison et al. Diabetes Care, 25(6), 961, 2002.

When blood glucose is rapidly ramping (changing) either up (hyperglycemia) or down (hypoglycemia), the lag time becomes a critical issue for determining the response time for any external, non-invasive blood glucose monitor. Rapid response is required for identifying important health related changes in glucose levels and to avoid critical blood glucose scenarios (i.e., clinically important high or low blood glucose levels). Issues of rapid response are addressed by using elevated temperatures at the measurement site to increase blood flow to these regions. Therefore, in various embodiments, the sensor unit is combined with a regulatable heating element and/or temperature gauge. The sensors are calibrated by comparing actual blood glucose to the sensor output. The temperature is either controlled at the measurement site or compensated for in the final blood glucose estimation. Kₐ and φ_{F} are only slightly temperature dependent. The zero and slope of the sensor calibration are determined by measuring an initial baseline glucose level, and a second glucose level at higher concentration. The sensor calibration is then measured as shown in equation (26):

[G]=K₁(sensor response)+K₀ (26)

The K₁ and K₀ values are entered into the sensor and the calibration is checked against a reference standard material. The reference standard material is comprised of a matrix that responds to glucose concentration in such a way as to provide primary standard concentration and fluorescence response data. Their relationship is given in equation (27), where A, B, C, and D are comprised of one or more individual analyte measurements or ratios of measurements. The method shown in equations (26) and (27) can be used either for calibration using YSI determined blood reference data; or without blood reference data via use of equations (13) through (21).

Algorithm:

[G]=f([A],[B],[C],[D])* (27)

*Where [A], [B], [C], and [D] are directly measured using one or more measurement techniques for one or more metabolite signals. Each metabolite signal represents a mechanism for quantitatively measuring intracellular or extracellular glycolysis. The primary fuel for glycolysis is D-glucose combined with low concentrations of other simple sugars, such as galactose and fructose.

Equivalents

From the foregoing detailed description of the specific embodiments of the invention, it should be apparent that particular novel compositions and methods involving utilizing SMMRs for direct or indirect measurements of metabolic analyte concentrations have been described. Although these particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made as a matter of routine for a person of ordinary skill in the art to the invention without departing from the spirit and scope of the invention as defined by the claims. Indeed, various adaptations and modifications of the invention in addition to those described herein will become apparent to those skilled in the art from the foregoing description and accompanying figures. Such modifications are intended to fall within and be incorporated into the scope of the appended claims.

What is claimed is:

1. A method for measuring in vivo blood glucose levels through the skin, said method comprising monitoring, in a population of cells one or more relevant metabolites, parameters or analytes in at least one metabolic pathway, wherein the monitoring comprises measuring the fluorescence spectrum emitted by a reporter composition located in the skin, wherein the fluorescence spectrum emitted by the reporter is stoichiometrically related to the metabolite, parameter or analyte concentration in the population of cells, whereby analyzing the relatedness provides the in vivo blood glucose level.

2. The method of claim 1, wherein the population of cells has a predominantly glycolytic metabolism or can be induced to have a glycolytic metabolism.

3. The method of claim 2, wherein the population of cells in the skin is located in the epidermis, wherein the epidermis comprises a dynamic, metabolically homogeneous, and homeostatic population of cells.

4. The method of claim 2, wherein the population of cells having a glycolytic metabolism comprise live keratinocytes.

5. The method of claim 4, wherein the live keratinocytes are present in the epidermal layer of skin.

6. The method of claim 5, wherein the live keratinocytes are present at a depth from the surface of the skin from about 10 μm, wherein said depth corresponds with the bottom of the dead stratum corneum layer, to about 175 μm, wherein said depth corresponds with the top of the dermal layer.

7. The method of claim 1, wherein the metabolic pathway is monitored within the population of cells via measurement of a specific metabolite or analyte of the glycolytic pathway that has a stoichiometric or highly correlated relationship with glucose concentration.

8. The method of claim 1, wherein the metabolic pathway is monitored within the population of cells, via a physico-chemical parameter that is related to the glycolytic pathway, wherein said parameter has a stoichiometric or highly correlated relationship with glucose concentration.

9. The method of claim 7, wherein the one or more relevant metabolites or analytes are selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; inorganic phosphate (Pᵢ); glycogen; pyruvate; nicotinamide adenine dinucleotide phosphate, oxidized form (NAD(P)+); nicotinamide adenine dinucleotide (phosphate), reduced form (NAD(P)H); flavin adenine dinucleotide, oxidized form (FAD); flavin adenine dinucleotide, reduced form (FADH₂); and oxygen (O₂) utilization.

10. A skin sensor composition, comprising one or more of:
a reporter dye and a marker dye; or
a dye exhibiting a wavelength shift in absorption or fluorescence emission in the presence of a metabolite;
wherein the skin composition is present at a depth from the surface of the skin from about 10 μm, wherein said depth corresponds with the bottom of the dead stratum corneum layer, to about 175 μm, wherein said depth corresponds with the top of the dermal layer, in the epidermis at an effective concentration for detection of one or more metabolites or analytes in a metabolic pathway in a subject or biological sample.

11. The skin composition of claim 10, wherein the reporter dye is chosen from the group consisting of: a mitochondrial vital stain or dye, and a dye exhibiting one or more of a redox potential, an energy transfer properties, and a pH gradient.

12. The skin composition of claim 11, wherein the mitochondrial vital stain or dye is a polycyclic aromatic hydrocarbon dye selected from the group consisting of: rhodamine 123; di-4-ANEPPS; di-8-ANEPPS; DiBAC₄(3); RH421; tetramethylrhodamine ethyl ester, perchlorate; tetramethylrhodamine methyl ester, perchlorate; 2-(4-(dimethylainino)styryl)-N-ethylpyridinium iodide; 3,3′-dihexyloxacarbocyanine, 5,5′,6,6′-tetrachloro-1,1′,3,3′-tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; and iodine dissolved in potassium iodide.

13. The skin composition of claim 10, wherein the reporter dye is selected from the group consisting of: coumarin; derivatives of coumarin, anthraquinones; cyanine dyes, azo dyes; xanthene dyes; arylmethine dyes; pyrene derivatives; and ruthenium bipyridyl complexes.

14. The skin composition of claim 10, wherein the one or more metabolites or analytes is selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); flavin adenine dinucleotide, reduced form (FADH₂); and oxygen (O₂) utilization.

15. The skin composition of claim 10, wherein the effective concentration is selected from the group consisting of at least between 1 to 500 μg/ml, between 5 to 150 μg/ml, and 10 to 100 μg/ml.

16. The skin composition of claim 15, wherein a specific application comprises a 5 μL volume of a 400 μM SMMR solution, or a 10 μL volume at 200 μM concentration.

17. The skin sensor composition of claim 10, wherein the one or more metabolites or analytes directly report on and relate to in vivo blood glucose levels.

18. The skin sensor composition of claim 17, wherein the related metabolites or analytes are selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); flavin adenine dinucleotide, reduced form (FADH₂); and oxygen (O₂) utilization.

19. A method for monitoring the concentration of one or more metabolites or analytes, the method comprising:
applying the skin sensor composition according to claim 10 to a surface of the skin for a predetermined period of time;
causing penetration of the skin sensor composition to a depth of about 10 μm, wherein said depth corresponds with the bottom of the dead stratum corneum layer, to about 175 μm, wherein said depth corresponds with the top of the dermal layer, into the epidermis; and
monitoring a change in the concentration of the one or more metabolites or analytes in a metabolic pathway by detecting changes in one or more reporter dyes at one or more time points using an optical reader.

20. The method of claim 1, wherein the population of cells has a predominantly oxidative metabolism or can be induced to have a metabolism predominantly based on oxidative phosphorylation.

21. The method of claim 20, wherein the metabolic pathway is monitored within the population of cells via a metabolite or analyte that is generated as a result of the oxidative metabolic pathway and that has a stoichiometric or highly correlated relationship with glucose concentration.

22. The method of claim 20, wherein the metabolic pathway is monitored within the population of cells via a physico-chemical parameter that is generated as a result of the oxidative metabolic pathway and that has a stoichiometric or highly correlated relationship with glucose concentration.

23. The method of claim 19, wherein the skin sensor composition comprises a mitochondrial stain sensitive to membrane potential or chemical gradient.

24. The method of claim 19, wherein the skin sensor composition comprises a dye or stain that transfers energy from a molecule generated as a result of the oxidative metabolic pathway and that has a stoichiometric or highly correlated relationship with glucose concentration.

25. The method of claim 23, wherein the mitochondrial stain is a polycyclic aromatic hydrocarbon dye selected from the group consisting of: rhodamine 123; di-4-ANEPPS; di-8-ANEPPS; DiBAC₄(3); RH421; tetramethylrhodamine ethyl ester, perchlorate; tetramethylrhodamine methyl ester, perchlorate; 2-(4-(dimethylamino)styryl)-N-ethylpyridinium iodide; 3,3′-dihexyloxacarbocyanine, 5,5′,6,6′-tetrachloro-1,1′,3,31-tetraethyl-benzimidazolylcarbocyanine chloride; 5,5′,6,6′-tetrachloro-1,1′,3,3′-tetraethyl-benzimidazolylcarbocyanine iodide; nonylacridine orange; dihydrorhodamine 123 dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; and iodine dissolved in potassium iodide.

26. The method of claim 19, wherein the skin sensor composition comprises a dye selected from the group consisting of: coumarin; derivatives of coumarin; anthraquinones; cyanine dyes; azo dyes; xanthene dyes; arylmethine dyes; pyrene derivatives; and ruthenium bipyridyl complexes.

27. The method of claim 19, wherein the one or more metabolites or analytes is selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; inorganic phosphate (Pᵢ); glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); and flavin adenine dinucleotide, reduced form (FADH₂); and oxygen (O₂) utilization.

28. The method of claim 19, wherein the skin sensor composition is formulated as any one or more of the following: an emulsion, an ointment, a disposable gel film patch, a reservoir device, a cream, a paint, polar solvents or non-polar solvents.

29. The method of claim 19, wherein the penetration of the skin composition is accomplished using an active transport technique or a passive transport technique selected from the group consisting of: electroporation, laser poration, sonic poration, ultrasonic poration, iontophoresis, mechanical-poration, solvent transport, tattooing, wicking, and pressurized delivery.

30. The method of claim 19, wherein the penetration of the skin sensor composition to a depth of about 10 μm to about 175 μm is accomplished by combining the composition with molecular size attachments.

31. The method of claim 19, where the predetermined period of time is selected from the group consisting of at least 24-48 hours, at least 2-6 hours, from about 5 seconds to 5 minutes, and from about 30 seconds to 5 minutes.

32. The method of claim 19, where monitoring the change in metabolite or analyte concentration comprises detecting at least one wavelength above 450 nm.

33. A method for monitoring in vivo blood glucose levels, the method comprising:
applying the skin sensor composition according to claim 10 to a surface of the skin for a predetermined period of time;
causing penetration of the skin sensor composition to a depth of about 10 μm, wherein said depth corresponds with the bottom of the dead stratum corneum layer, to about 175 μm, wherein said depth corresponds with the top of the dermal layer, into the epidermis;
monitoring a change in the concentration of the one or more metabolites or analytes by detecting changes in the reporter dye using an optical reader, and
correlating the change in the concentration of the one or more metabolites or analytes with in vivo blood glucose levels.

34. The method of claim 33, wherein the skin sensor composition comprises a mitochondrial vital stain or dye, or a dye exhibiting redox potential or energy transfer properties.

35. The method of claim 34, wherein the mitochondrial vital stain or dye is at least one polycyclic aromatic hydrocarbon dye selected from the group consisting of: Rhodamine 123, Di-4-ANEPPS; Di-8-ANEPPS, DiBAC₄(3), RH421, Tetramethylrhodamine ethyl ester, perchlorate, Tetramethylrhodamine methyl ester, perchlorate, 2-(4-(dimethylamino)styryl)-N-ethylpyridinium iodide, 3,3′-Dihexyloxacarbocyanine, 5,5′,6,6′-tetrachloro-1,1′,3,3′-tetraethyl-benzimidazolylcarbocyanine chloride, 5,5′,6,6′-tetrachloro-1,1′,3,3′-tetraethyl-benzimidazolylcarbocyanine iodide, Nonylacridine Orange, Dihydrorhodamine 123 and Dihydrorhodamine 123, dihydrochloride salt; xanthene; 2′,7′-bis-(2-carboxyethyl)-5-(and-6)-carboxyfluorescein; benzenedicarboxylic acid; 2(or 4)-[10-(dimethylamino)-3-oxo-3-H-benzo[c]xanthene-7-yl]; and iodine dissolved in potassium iodide.

36. The method of claim 33, wherein the skin sensor composition comprises at least one dye selected from the group consisting of: coumarin, derivatives of coumarin, anthraquinones, cyanine dyes, azo dyes, xanthene dyes, arylmethine dyes, pyrene derivatives, and ruthenium bipyridyl complexes.

37. The method of claim 33, wherein the one or more metabolites or analytes is selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); flavin adenine dinucleotide, reduced form (FADH₂); and oxygen (O₂) utilization.

38. The method of claim 33, wherein the skin sensor composition is formulated as an emulsion, cream, ointment, disposable gel film patch, reservoir device, paint, or solvent mixture.

39. The method of claim 33, wherein the penetration of the skin composition is accomplished using at least one active transport or passive transport technique selected from the group consisting of: electroporation, laser poration, sonic poration, ultrasonic poration, solvent transport, iontophoresis, mechanical-poration, tattooing, painting, wicking and pressurized delivery.

40. The method of claim 33, wherein the penetration of the skin sensor composition to a depth of about 10 μm, wherein said depth corresponds with the bottom of the dead stratum corneum layer, to about 175 μm, wherein said depth corresponds with the top of the dermal layer, is accomplished by combining the composition with molecular size attachments.

41. The method of claim 33, where the predetermined period of time is selected from the group consisting of at least 24-48 hours, at least 2-6 hours, from about 5 seconds to 5 minutes, and from about 30 seconds to 5 minutes.

42. The method of claim 33, where monitoring the change in the one or more metabolite or analyte concentrations comprises measuring at least one spectral emission at a wavelength above 450 nm.

43. The method of claim 33, wherein the one or more metabolites are selected from the group consisting of: lactate; hydrogen ion (H⁺); calcium ion (Ca²⁺) pumping rate; magnesium ion (Mg²⁺) pumping rate; sodium ion (Na⁺) pumping rate; potassium ion (K⁺) pumping rate; adenosine triphosphate (ATP); adenosine diphosphate (ADP); the ratio of ATP to ADP; glycogen; pyruvate; nicotinamide adenine dinucleotide phosphate, oxidized form (NAD(P)+); nicotinamide adenine dinucleotide phosphate, reduced form (NAD(P)H); flavin adenine dinucleotide, oxidized form (FAD); flavin adenine dinucleotide, reduced form (FADH₂); and oxygen (O₂) utilization.

44. A sensor system, the system comprising:
a device comprising a component that transmits radiation to a material or tissue, a component that detects radiation emitted from a material or tissue, and a component to display the detection results;
an applicator that delivers the skin sensor composition of claim 10 to the material or tissue; and
an air interface between the device and the material or tissue, wherein the air interface measures a resulting excitation radiation emitted from the irradiated skin sensor composition.

45. The sensor system of claim 44, wherein said system comprises a device that emits radiation at one or more wavelengths chosen to specifically excite the skin composition that is applied to the material or tissue, wherein the skin sensor composition comprises one or more of:
a reporter dye and a marker dye; or
a dye exhibiting a wavelength shift in absorption or fluorescence emission in the presence of a metabolite;
wherein the skin sensor composition is present at a depth from the surface of the skin of about 10 μm, wherein said depth corresponds with the bottom of the dead stratum corneum layer, to about 175 μm, wherein said depth corresponds with the top of the dermal layer, in the epidermis at an effective concentration for detection of one or more metabolites or analytes in a biological sample.

46. The sensor system of claim 44, wherein said system detects radiation at one or more wavelengths chosen to specifically identify fluorescence emission scattered back to the system from the skin sensor composition.

47. A method for determining blood glucose concentration, comprising the steps of:
performing an instrument response measurement on a calibration target and recording the response data;
applying a dye mixture to the skin in a first small controlled spot such that the dye resides in the epidermal layer of the skin;
applying a second dye mixture to the skin in a second small controlled spot and perturbing the second spot such that one or more extreme changes that the mixture may undergo are achieved;
performing a calibration measurement on the perturbed spot and recording the calibration data;
performing a background measurement on an area of skin that has no dye and recording this background data;
performing a measurement on the first spot by illuminating the first spot with light;
detecting wavelength spectrum of light reflected back from the first spot;
performing further measurements on the first spot at wavelengths suitable for each dye present;
calculating a parameter from the response data to normalize the background, calibration and measurement data for the response of the spectrometer;
calculating a parameter from the background data to correct the calibration and measurement data for emission, absorption and scattering properties of the tissue;
calculating a metabolite parameter from the calibration data to relate the measurement data to the blood glucose concentration.

48. The method of claim 47, wherein the one or more extreme changes is a change in concentration of the metabolite or analyte between a zero or low concentration and a saturation level or high concentration.

49. A method of calculating a blood glucose concentration, said method comprising:
measuring a background response and an autofluorescence tissue response from a calibration target comprising an epidermal layer of skin;
providing a first dye to a first skin location and causing residues of the first dye mixture to transfer into the epidermal layer of the skin;
providing a second dye to a second skin location and causing and recording at least one extreme change in the mixture;
illuminating the first skin location with a radiative emission;
detecting a resulting wavelength spectrum reflected from the first skin location;
optionally repeating the illuminating and detecting steps using irradiation and wavelength spectra associated with each dye provided; and
detecting at least one physico-chemical parameter that is related to the glycolytic pathway, wherein said parameter comprises a stoichiometric or highly correlated relationship with glucose concentration;
thereby determining the blood glucose concentration.

50. The method of claim 49, wherein the sensor system comprises a bloodless calibration procedure as outlined in one or more of equations 13, 16, 17, 18, 19, 20 or 21.

51. The method of claim 49, wherein the at least one extreme change is a change in the blood glucose concentration between a zero or low concentration and a saturation level or high concentration.

52. A method for determining the concentration of at least one metabolite or analyte in skin tissue, the method comprising:
(a) administering to the skin tissue a small molecule metabolite reporter (SMMR) agent;
(b) causing penetration of the SMMR agent to a region of the skin at a depth between the dermis and the epidermis, wherein the depth from the surface of the skin is from about 10 μm, wherein said depth corresponds with the bottom of the dead stratum corneum layer, to about 175 μm, wherein said depth corresponds with the top of the dermal layer;
(c) irradiating the SMMR agent in the skin tissue with a source of electromagnetic radiation;
(d) measuring the fluorescence spectra emitted from the SMMR agent; and
(e) analyzing the emitted fluorescence spectra;
wherein the analysis will result in a determination of the concentration of the metabolite or analyte.

53. The method of claim 50, wherein the measuring of the fluorescence spectra comprises a bloodless calibration procedure as outlined in one or more of equations 13, 16, 17, 18, 19, 20 and 21.
